(19) United States
(12) Patent Application Publication
(10) Pub. No.: US 2006/0258582 A1
(43) Pub. Date: Nov. 16, 2006

(54) Method of treating myelodysplastic syndromes

(75) Inventors: Mario A. Navas, San Francisco, CA (US); Linda S. Higgins, Palo Alto, CA (US)

Correspondence Address: MORRISON & FOERSTER LLP, 12531 HIGH BLUFF DRIVE, SUITE 100, SAN DIEGO, CA, 92130-2040, US

(73) Assignee: Scios, Inc., Fremont, CA (US)

(21) Appl. No.: 11/267,813

(22) Filed: Nov. 4, 2005

Related U.S. Application Data
(60) Provisional application No. 60/625,626, filed on Nov. 4, 2004.
(60) Provisional application No. 60/633,116, filed on Dec. 3, 2004.

(51) Int. Cl. A61K 38/54; G01N 33/574

(52) U.S. Cl. 514/12; 435/7.23

(57) ABSTRACT

The disclosed invention is directed to methods and compounds useful in treating a myelodysplastic syndrome (MDS) using p38 MAP kinase inhibitors either alone or in combination with other chemotherapeutic compounds. A role for p38 kinase inhibition as a treatment modality for combating MDS is discussed herein. Relating to a preferred embodiment, compounds of the invention have been found to inhibit p38 kinase, the α-isoform in particular, and are useful in treating MDS.

### RELATED APPLICATIONS

This application claims the benefit of priority of U.S. Provisional Application No. 60/625,626, filed Nov. 4, 2004 and U.S. Provisional Application No. 60/633,116 filed Dec. 3, 2004, both of which are hereby incorporated by reference in their entirety.

### TECHNICAL FIELD

The disclosed invention relates to the inhibition of p38 MAPK activity as a treatment of Myelodysplasia syndromes (MDS).

### BACKGROUND ART

There were approximately 13,000 cases of MDS reported in the United States in 1999. The risk of developing the syndrome increases with age and most subjects with MDS are 60 years of age or older. However, childhood onset of the syndrome is known in the art. As a rule, patients with MDS present with refractory anemia or are asymptomatic. Complications of MDS include infection due to cytopenia, hemorrhage, iron overload, alloimmunization, fatigue, and acute leukemia. Three different subtypes of MDS (low, intermediate and high risk) are now recognized, depending on the propensity of the condition to progress to acute leukemia.

The French-American-British cooperative group classification system (FAB classification) classifies MDS into five groups. These groups are refractory anemia (RA), RA with ringed sideroblasts (RARS), RA with excess blasts (RAEB), RA refractory anemia with excess blasts in transformation (RAEB-t), and chronic myelomonocytic leukemia (CMML). Table 1 below provides a number of characteristics associated with each category of MDS.

TABLE 1

| | | % Blasts | | | | |
|---|---|---|---|---|---|---|
| | | Peripheral | Bone | Ringed | Monocytosis | Median |
| Category | Grade | blood | marrow | Sideroblasts (%) | (>1 × 10⁹/L) | Survival (mo) |
| RA | low | <1 | <5 | <15 | absent | 50 |
| RARS | low | <1 | <5 | >15 | absent | 51 |
| RAEB | low | <5 | 5-20 | variable | absent | 11 |
| RAEB-t | high | >5 | 21-29 | variable | variable | 5 |
| CMML | high | <1 | <20 | variable | present | 11 |

The etiology of myelodysplastic syndromes (MDS) is unknown. However, it is thought that MDS results from abnormalities in hematopoietic stem cell. Clinical features of MDS include ineffective hematopoiesis, exemplified by variable anemia, leukopenia, and thrombocytopenia. Additionally, subjects with MDS present a paradoxical hypercellular marrow. MDS marrow shows both an increase in cellular proliferation and bone marrow cell apoptosis. Peripheral blood cells thus produced typically possess a number of abnormalities, including normoblast with nuclear irregularities, basophilic stippling, bibbed Pelger-Huet like neutrophils, giant platelets, Howell-Jolly body cells, acanthocytes, and granulocytes with abnormal nucleation and granularity. Moreover, bone marrow in a MDS patient will often show signs medullary neovascularization. In addition to the pathological features of ineffective hematopoiesis, individuals with MDS run the risk of transformation to acute leukemia. MDS patients typically present with primarily refractory anemia, and depending on the stage or type of disease, MDS patients will have a very cellular bone marrow populated by MDS cancer clones referred to as “blasts”.

Myelodysplasia syndromes (MDS) are hematopoietic disorders known by a wide variety of names. Examples of terms used to describe syndromes falling under the rubric of myelodysplasia include preleukemia, refractory anemia with excess of myeloblasts, subacute myeloid leukemia, oligoleukemia, odoleukemia and dysmyelopoietic syndromes.

Myelodysplasia syndromes (MDS) are clonal stem cell disorders. Clinical features of the syndromes include progressive deficiencies of one or more blood cell types (cytopenia) and the presence of a hypercellular bone marrow. MDS are typically rare and acute in children. More typically, MDS manifest in the elderly, especially those who have received chemotherapy or radiotherapy. Myelodysplasia syndromes tend to evolve into acute nonlymphocytic leukemias (ANLL), however, not all cases terminate in leukemia.

The cellular elements of blood, both myeloid and lymphoid, are produced from a self-renewing, pluripotent stem cell. The pluripotent stem cell first differentiates into a committed stem cell and then into either a myeloid progenitor or a lymphoid progenitor. The myeloid progenitor produces erythrocytes (red blood cells), platelets, granulocytes, monocytes, dendritic cells and mast cells or basophils. The panoply of conditions subsumed by the term MDS result from the dysregulation of progenitor cells in the myeloid line. As such, a subject suffering from a MDS undergoes bone marrow failure due to improper hematopoiesis as opposed to a lack of hematopoiesis.

A number of chromosomal abnormalities have been linked to MDS. Typically, these abnormalities involve chromosomes 5, 7, and 8. Studies suggest the loss of function of a tumor suppressor gene within a deleted segment of chromosome 7 as a possible contributing factor to MDS. Mutation or chromosome damage may result from a germline mutation or may be acquired from cytotoxic therapy, such as chemotherapy or radiotherapy.

Clinically, ineffective hematopoiesis is manifested as isolated anemia, neutropenia, or thrombocytopenia, or multiple cytopenias. Often, an isolated cytopenia progresses to pancytopenia over a period of weeks to months.

### DISCLOSURE OF THE INVENTION

The disclosed invention is directed to methods useful in treating a myelodysplastic syndrome (MDS) using p38 MAP kinase inhibition. More specifically, the disclosed invention relates to compounds and methods of using same comprising the administration of one or more p38 MAPK inhibitory compounds either alone or in combination with other chemotherapeutic compounds. A role for p38 kinase inhibition as a treatment modality for combating MDS is discussed herein. Relating to a preferred embodiment, compounds of the invention have been found to inhibit p38 kinase, the α-isoform in particular, and are useful in treating MDS. Preferred examples of the compounds of the invention are of the formula:

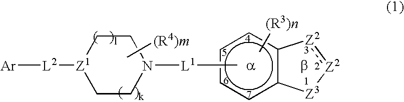

and the pharmaceutically acceptable salts thereof, or a pharmaceutical composition thereof, wherein 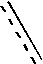
represents a single or double bond;

one Z² is CA or CR⁸A and the other is CR¹, CR¹₂, NR⁶ or N wherein each R¹, R⁶ and R⁸ is independently hydrogen or noninterfering substituent;

A is —Wᵢ—COXⱼY wherein Y is COR² or an isostere thereof and R² is hydrogen or a noninterfering substituent, each of W and X is a spacer preferably 2-6 Å in length, and each of i and j is independently 0 or 1;

Z³ is NR⁷ or O;

each R³ is independently a noninterfering substituent;

n is 0-3;

each of L¹ and L² is a linker;

each R⁴ is independently a noninterfering substituent;

m is 0-4;

Z¹ is CR⁵ or N wherein R⁵ is hydrogen or a noninterfering substituent;

each of l and k is an integer from 0-2 wherein the sum of l and k is 0-3;

Ar is an aryl group substituted with 0-5 noninterfering substituents, wherein two noninterfering substituents can form a fused ring; and

the distance between the atom of Ar linked to L² and the center of the α ring is preferably less than 24 Å.

### BRIEF DESCRIPTION OF THE DRAWINGS

FIG. 1A-C show histochemical images of normal bone marrow (3) and MDS cells (3) comparing levels of p38 MAPK activation, HSP27 phosphorylation and caspase 3 activation.

FIG. 2A-C shows bar graphed comparisons of p38 MAPK activation, HSP27 phosphorylation and caspase 3 activation from normal bone marrow and bone marrow from an MDS patient.

FIG. 3 shows flow cytometric analysis indicating that increased p38 MAPK phosphorylation correlates with increased IL-1β expression and caspase 3 activation of bone marrow cells from low risk MDS patients. N1-N3 show normal samples. RL, BB, AH, DC and BG show samples from patients with MDS.

FIG. 4A-D shows a bar graph (A), a plot of phosphorylated p38 MAPK (˜p-p38) vs. IL-1β (B), a line graph (C) and a plot of phosphorylated p38 MAPK (˜p-p38) vs. caspase 3 (D), which represent the statistical correlation showing that increased phosphorylated p38 MAPK increases with IL-1β expression and caspase 3 activation of bone marrow cells in low risk MDS patients.

FIG. 5 shows gel electrophoretic results of p38 MAPK activity.

FIG. 6 is a schematic representation of the interaction of p38 MAPK activity with various cytokines relevant to MDS. Information for the preparation of this figure was derived from Hsu, et al. J Biol Chem. (1999) 274(36):25769-76, Porras, et al., Mol Biol Cell. (2004) 15(2):922-33, and Grambihler, et al. (2003) J Biol Chem. 2003 Jul. 18; 278(29):26831-7.

FIG. 7 shows gel electrophoretic results of p38 MAPK activity in the presence of TNFα, TGFβ and a p38 MAPK inhibitor.

FIG. 8 shows cell viability counts by GUAVA VIACOUNT in the presence of various concentration of a p38 MAPK inhibitor and MTS assay where the cells were grown in the presence of a single concentration of a p38 MAPK inhibitor, TNFα, or TGFβ.

FIG. 9 shows the results of various cytokine array assays examining the cytokines produced by bone marrow stromal cells (BMSC) in the presence of MDS cells, TNF, a p38 MAPK inhibitor, or combinations of these agents.

FIG. 10 shows bar graphs indicating the expression levels of cytokines (IL-1β, VEGF, TNF-, and IL-6) produced by BMSC, BMMNC, combinations of the same, MDS and a p38 MAPK inhibitor.

FIG. 11 shows a cell viability plot using GUAVA VIACOUNT and MDS bone marrow cells in the presence and absence of a p38 MAPK inhibitor.

FIG. 12A-D shows the positive effects of p38 MAPK inhibitors on erythroid and myeloid colonies.

FIG. 13 shows portions of a SDS-PAGE gel. BM derived CD34+ progenitors at the CFU-Erythroid stage of maturation were treated with 20 ng/ml TNFα in the presence and absence of 100 nM compound 57. Cell lysates were resolved by SDS-PAGE and immunoblotted with an antibody against the phosphorylated form of MapKapK-2 on threonine 334. The same blot was stripped and re-probed with an antibody against MapKapK-2, to control for protein loading.

FIG. 14A-B shows cell plots indicating that a p38 MAPK inhibitor is effective to inhibit TNFα-induced apoptosis of normal CD34+ progenitors. (a) Primary bone marrow derived CD34+ cells were grown in cytokine enriched liquid media in the presence and absence of 20 ng/ml TNFα and compound 57 (100 nM). The percentage of apoptotic and dead cells were determined by staining with a mixture of Annexin V-Alexa Fluor 488 and nucleic acid dye, Sytox green respectively (VYBRANT APOPTOSIS KIT, Molecular Probes). (b) Mean of three independent experiments showed significant decrease in TNFα-mediated apoptosis of normal progenitors in the presence of compound 57.

FIG. 15 shows a bar graph indicating exposure to a p38 MAPK inhibitor reverses the TNFα-induced myelosuppression of normal CD34+ progenitors. Primary bone marrow derived CD34+ cells were cultured in methylcellulose in the presence and absence of 20 ng/ml TNFα and with the indicated concentrations of compound 57 (nM). BFU-E and CFU-GM colonies were scored on Day 14. Results are expressed as means+/−S.E.M. of three independent experiments.

FIG. 16 shows a bar graph indicating exposure to a p38 MAPK inhibitor reverses the IFNγ-induced myelosuppression of normal CD34+ progenitors. Primary bone marrow derived CD34+ cells were cultured in methylcellulose in the presence and absence of Xng/ml IFNγ and with the indicated concentrations of the p38α inhibitors (compound 57; nM) or compound 162. BFU-E and CFU-GM colonies were scored on Day 14. Results are expressed as means+/−S.E.M. of three independent experiments.

FIG. 17 A-D show graphical cell scattering results (A-C) and a bar graph (D) indicating that a p38 MAPK inhibitor decreased apoptosis of CD34+ progenitors in MDS BMMNC cell culture. In (A-C) BM mononuclear cells from three different patients with low risk MDS were cultured in the presence and absence of 500 nM compound 57 for 48 hours. Apoptosis in gated population of CD34+ cells was determined by Annexin V-PE and propidium iodide staining. Three representative independent experiments demonstrate a decrease in the percentage of Annexin V positive CD34+ cells in samples treated with compound 57. In (D) MDS CD34+ progenitors from six patients demonstrate significantly greater viability and decreased apoptosis after 48 hours treatment with 500 nM compound 57.

FIG. 18 shows a bar graph indicating that a p38 MAPK inhibitor dose-dependently enhances erythroid and myeloid colony formation in MDS CD34+ progenitors. MDS BM-derived CD34+ cells from 19 patients with MDS (Table 5) were cultured in methylcellulose in the presence and absence of increasing concentrations of compound 57. Results are expressed as means+/−SEM of 19 independent experiments.

FIG. 19A-B show graphs indicating that exposure to a p38 MAPK inhibitor stimulates myeloid and erythroid colony formulation in isolated CD34+ progenistors from 19 MDS patients. (A) Total colony numbers of (A) Blast Forming unit-erythroid (BFU-E) and (B) Colony forming unit-Granulocytic Macrophage (CFU-GM) before (control) and after compound 57 treatment (100 nM) of 19 patients with MDS (Table 5).

FIG. 20 shows a bar graph indicating that a p38 MAPK inhibitor inhibits LPS-induced IL-1β expression in different populations of normal bone marrow. BMMNC (1×10⁶) were treated with or without 0.5 uM compound 57 and incubated in the presence or absence of 10 ng/ml LPS for 4 hours. Brefeldin (golgi plug) was added at a final concentration of 2 μg/ml during the last hour of incubation. Cells were harvested, washed and labeled with different fluorochrome conjugated antibodies to CD45 (leukocytes), CD14 (monocytes), CD3 (T cells), CD19 (B cells), CD56 (NK cells) and CD34 (progenitor cells) followed by intracellular staining with PE-conjugated anti-IL-1β. Figure shows the relative IL-1β expression for each of the specific BM populations. Results are expressed as means+/−S.D. of three independent experiments.

FIG. 21 A-D shows plots of cells labeled with PE-conjugated anti-IL-1β antibodies. BMMNC (1×10⁶) were treated with or without 10 ng/ml LPS and incubated in the presence or absence of increasing concentrations of Compound 57 for 4 hours. Brefeldin (golgi plug) was added at a final concentration of 2 μg/ml during the last hour of incubation. Cells were harvested, washed and labeled with different fluorochrome conjugated antibodies CD14 (monocytes), CD56 (NK cells) and CD34 (progenitor cells) followed by intracellular staining with PE-conjugated anti-IL-1β. Figure shows the relative IL-1β expression for each of the specific BM populations: CD14+ cells (green), CD34+ cells (light blue), CD56+ cells (violet).

FIG. 22 A-H shows cell plots. Primary bone marrow derived CD14+ cells were incubated in IMDM+10% FBS in the presence or absence of 20 ng/ml LPS and compound 57 for 4 hour. Brefeldin (golgi plug) was added at a final concentration of 2 μg/ml during the last hour of incubation. Cells were harvested, washed with FBS staining buffer and labeled with anti-CD14-Per CP Cy5.5 followed by intracellular staining with PE-conjugated anti-IL-1β and FITC-conjugated anti-TNFα. Figure shows % double-stained CD14+TNFα+ (left) and CD14+ IL1β+(right) in the same cell population.

FIG. 23 shows a bar graph plotting TNFα production against BMMNC cells exposed to p38MAPK inhibitor. BMMNC (1×10⁶) from a normal healthy donor were cultured in the absence and presence of increasing concentrations of compound 57 for 24 hours without or with 10 ng/ml LPS. TNFα concentration in cell supernatants were determined by ELISA. Figure represents Mean+/−SD of three independent experiments.

FIG. 24 shows a bar graph indicating the inhibitory impact of a p38MAPK inhibitor on IL-1β-induced TNFα expression in different cell populations of normal BMMNC. BMMNC (1×10⁶) were incubated without or with increasing concentrations of compound 57 and in the presence or absence of 50 ng/ml IL-1, for 24 hours. Brefeldin (golgi plug) was added to a final concentration of 2 μg/ml during the last 2 hours of incubation. Cells were harvested, washed, labeled and then fixed with different fluorochrome conjugated antibodies to CD45 (leukocytes), CD14 (monocytes), CD3 (T cells), CD19 (B cells), CD56 (NK cells) and CD34 (progenitor cells) followed by intracellular staining with PE-conjugated anti-TNFα. Figure shows the relative TNFα expression for each of the specific BM populations. Results are expressed as means+/−S.D. of three independent experiments.

FIG. 25A-F shows cells plots (A-D) and bar graph (E-F) indicating LPS-induced CD34+ apoptosis in normal BMMNC is inhibited by a p38MAPK inhibitor in vitro. BMMNC (1×10⁶) from a normal healthy donor were cultured in the absence and presence of increasing concentrations of compound 57 without or with 10 ng/ml LPS for 48 hours. Cells were stained with anti-CD34-PeCy7, anti-CD45-APCCy7, Annexin V-FITC and 7AAD and analyzed by flow cytometry using the BD LSRII. Left panel shows representative dot plots of Annexin V vs. 7AAD. Right panels show relative % apoptotic/necrotic (top) and viable (bottom) CD34+ CD45-populations. Figures represents Mean+/−SD of three independent experiments.

FIG. 26 shows a bar graph plotting TNFα productions against exposure to p38MAPK inhibitor. TNFα concentration was measured by ELISA in cell supernatants collected from the experiment performed in FIG. 25. Figure represents Mean+/−SD of three independent experiments.

FIG. 27 shows a bar graph showing that a p38MAPK inhibitor inhibits TNF secretion from co-cultures of normal BMMNC and BMSC and BMSC from either normal control or MDS patients. BMSC derived from either normal healthy control or from low risk MDS patients were co-cultured for 3 days with BMMNC derived from a normal donor with or without 0.5 μM compound 57. TNFα concentration in cell supernatants were determined by ELISA. Figure represents Mean+/−SD of three independent experiments.

FIG. 28 A-F shows cell distribution plots indicating that a p38MAPK inhibitor inhibits TNF production from CD14+ monocytes in MDS BMMNC. BMMNC isolated from three different MDS patients were cultured in the presence or absence of 0.5 μM compound 57 for 24 hours. Cells were then stained extracellularly with anti CD14-PE and intracellularly with TNFα-APC before analyzing by flow cytometry. Figure represents Mean+/−SD of three independent experiments.

FIG. 29 shows a bar graph indicating that a p38 MAPK inhibitor reduces MCP-1 production from TNF-stimulated BMMNC. BMMNC (1×10⁶) from a normal healthy donor were stimulated with TNF in the presence or absence of increasing concentrations of compound 57 for 24 hours. MCP-1 concentration in cell supernatants were determined by ELISA. Figure represents Mean+/−SD of three independent experiments.

FIG. 30 A-B show bar graphs plotting the reduction of IL-6 and VEGF production from BMSC induced by a p38MAPK inhibitor in a dose dependent manner. BMSC from a normal healthy donor were cultured in the presence or absence increasing concentrations of compound 57 for 24 hours. IL-6 and VEGF concentration in cell supernatants were determined by ELISA. Figure represents Mean+/−SD of three independent experiments.

FIG. 31 A-B show bar graphs plotting the reduction of BMMNC-induced IL-6 and VEGF production from BMSC by a p38MAPK inhibitor. BMSC from a normal healthy donor were cultured in the presence or absence of BMMNC from a normal donor with DMSO or with increasing concentrations of compound 57 for 5 days. IL-6 and VEGF concentration in cell supernatants were determined by ELISA. Figure represents Mean+/−SD of three independent experiments.

FIG. 32 A-B show bar graphs plotting the reduction of VEGF production from BMSC derived from either normal healthy controls or MDS patients caused by a p38MAPK inhibitor. Levels of VEGF secretion from BMSC isolated from MDS patients were comparably lower than those from BMSC isolated from healthy normal controls. VEGF production in BMSC from either sources was effectively reduced by 0.5 μM compound 57 treatment after 2 days of cell culture. Figure represents Mean+/−SD of three independent experiments.

FIG. 33 shows a bar graph indicating that a p38MAP kinase inhibitor reduces IL-1β induced IL-6 secretion from BMMNC. BMMNC (1×10⁶) from a normal healthy donor were stimulated without or with 50 ng/ml IL-1β in the presence or absence of increasing concentrations of compound 57 for 48 hours. IL-6 concentration in cell supernatants was determined by ELISA. Figure represents Mean+/−SD of three independent experiments.

FIG. 34 shows a bar graph indicating that exposure to a p38MAPK inhibitor inhibits the synergistic production of IFN-γ by IL-12 and IL-18 in BMMNC. BMMNC (1×10⁶) from a normal healthy donor were stimulated with IL-12, IL-18 or both in the presence or absence of increasing concentrations of compound 57 for 24 h. IFN-γ concentration in cell supernatants was determined by ELISA. Figure represents Mean+/−SD of three independent experiments.

FIG. 35 shows a bar graph indicating that exposure to a p38 MAPK inhibitor reduces basal and TGFβ-induced MMP-2 production from BMMNC. BMMNC (1×10⁶) from a normal healthy donor were stimulated without or with 10 ng/ml TGF-β in the presence or absence of increasing concentrations of compound 57 for 48 hours. MMP-2 concentration in cell supernatants was determined by ELISA.

FIG. 36 shows a bar graph indicating that a p38 MAPK inhibitor reduces MMP-9 production from BMMNC. BMMNC (1×10⁶) from a normal healthy donor were treated without or with 10 ng/ml TGF-β in the presence or absence of increasing concentrations of compound 57 for 48 hours. MMP-9 concentration in cell supernatants was determined by ELISA.

### MODES OF CARRYING OUT THE INVENTION

The invention described herein relates to the use of p38 MAP kinase inhibitors, either alone, in combination with other p38 MAP kinase inhibitors, or in combination with other chemotherapeutic agents effective against myelodysplastic syndromes (MDS). Accordingly, inhibition of p38 MAP kinase activity has a number of direct and indirect effects on MDS cells that are therapeutically beneficial for patients suffering from MDS.

MDS

While the underlying pathology in MDS is unknown, the clinical features of these diseases result from dysregulated hematopoiesis. There is observed an increase in bone marrow cellular proliferation, apoptosis and increased abnormal cytokine responses. For example, elevated cytokine levels are observed in MDS patients. Examples of such elevated immunosuppressive cytokines include TNF-α as well as IL-1β, VEGF, TGF-β, and IFN-γ. Increased angiogenesis and microvasculature development is also frequently observed.

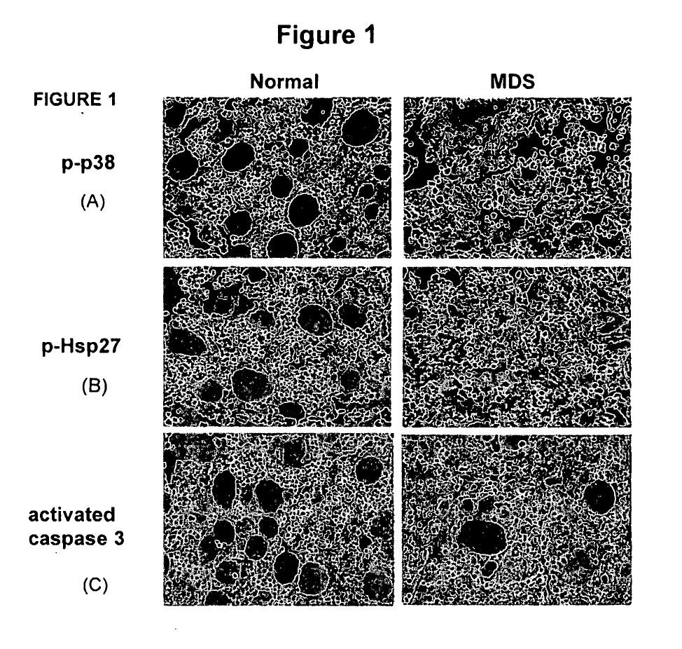

Elevated levels of activated p38 MAPK are seen in low risk MDS bone marrow samples taken from MDS patients as compared to the levels of p38 MAPK activated in normal bone marrow samples. This is illustrated in FIG. 1A, where phosphorylated p38 MAPK kinase, the activated form, is stained in both normal and MDS bone marrow samples. Thus, elevated levels of p38 MAPK activity are associated with MDS.

FIG. 1B shows that elevated levels of the phosphorylated form of the heat shock protein Hsp-27 are associated with MDS. The Hsp27 protein is a downstream marker of p38 MAPK activity.

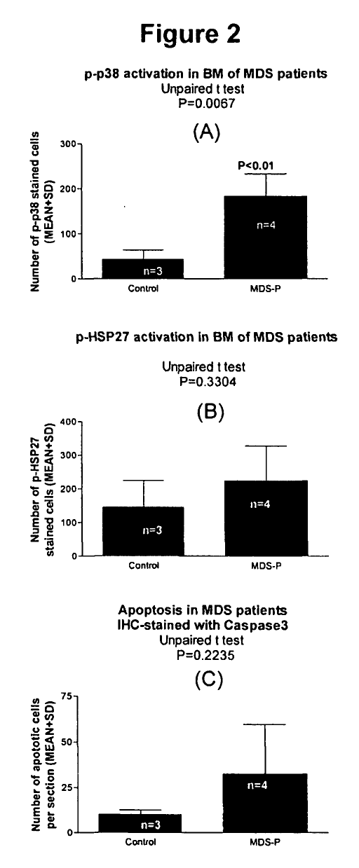

Apoptosis in MDS patients is also observed to be atypical. In early MDS proapoptotic stimuli predominate (mainly via Fas and TNFα). While in late MDS antiapoptotic stimuli predominate (mainly increased Bcl-2 and FLIP-L), thus blocking the release of cytochrome c. As shown in FIG. 1C, staining of activated caspase 3, a marker of apoptotic activity, is markedly increased in MDS bone marrow as compared to a normal bone marrow sample. FIGS. 2A-C shows a statistical comparison of the number and intensity of phosphorylated p38 MAPK, phosphorylated HSP27 and activated caspase 3 positive bone marrow cells in an MDS patient as compared to normal bone marrow.

A variety of cellular and biochemical features of bone marrow involved with MDS are discussed in Table I.

TABLE I

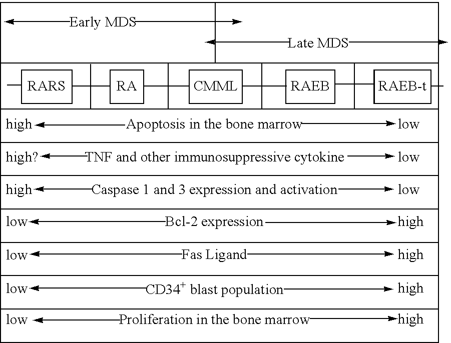

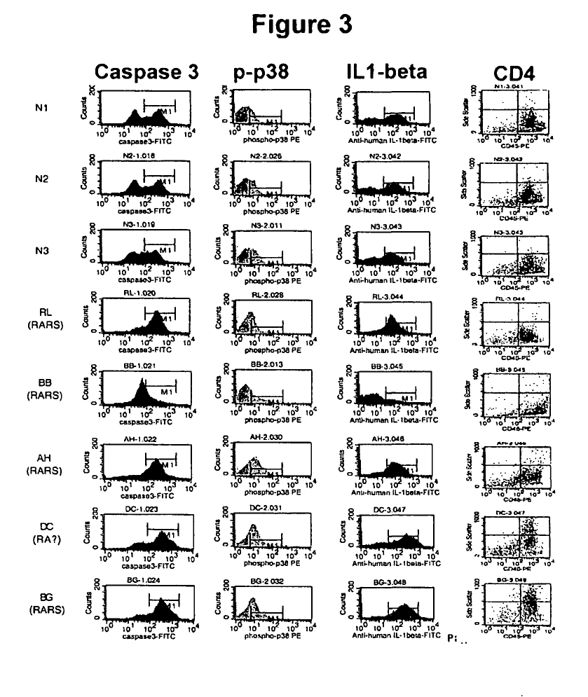

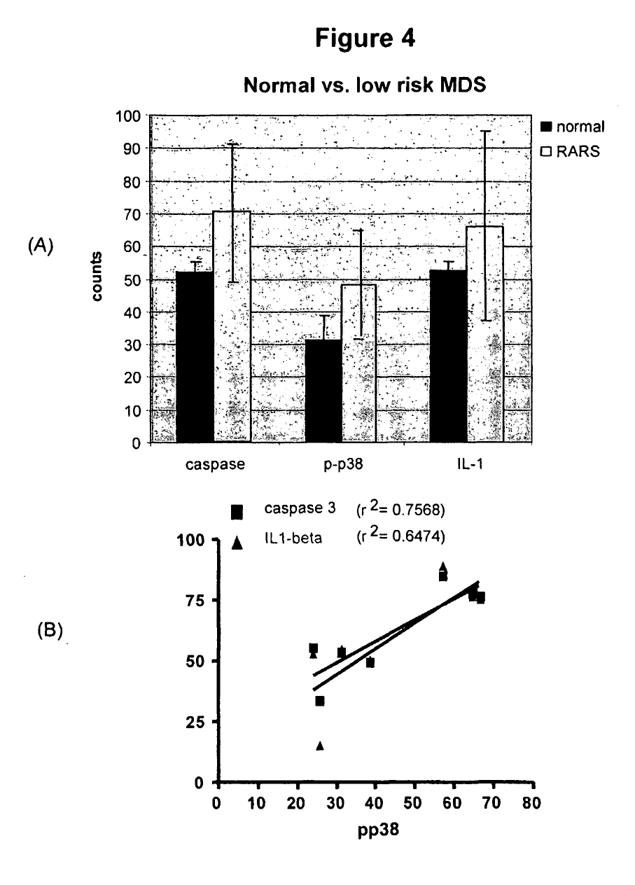

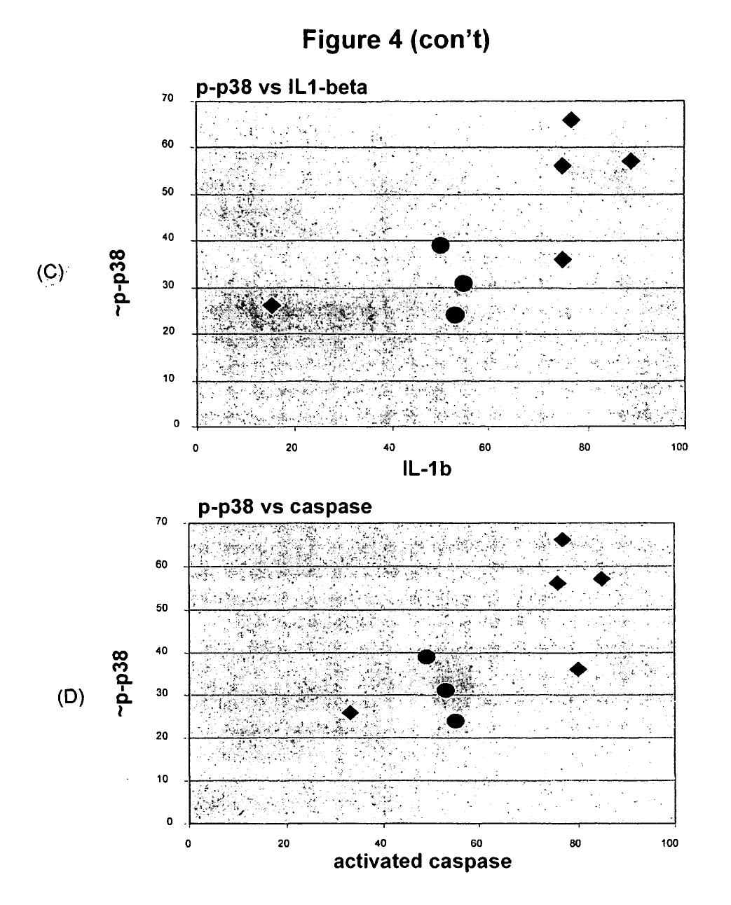

One of the indicia of MDS is an increased expression of cytokines in the bone marrow. One of the cytokines that shows an increase in expression is IL-1β. As shown in FIGS. 3 and 4, bone marrow cells from low risk MDS patients subjected to flow cytometry analysis shows that increased levels of p38 MAPK phosphorylation correlated with increased IL-1β expression and caspase 3 activation. Paraformaldehyde fixed normal or MDS bone marrow cells were permeabilized with either methanol prior to staining with caspase-3-FITC or p-p38-PE, or with detergent prior to staining with CD45-FITC and IL1-β-PE. IL1-β expression was determined only from CD45-gated cells.

MAP Kinase Inhibitors Cytokines and MDS

Mitogen-activated protein kinases (MAPKs) are activated by tyrosine and threonine phosphorylation. The disclosed invention has utility in treating MDS by modulating MAPKs in to reduce the negative affects of cytokines, especially immunosuppressive cytokines, on normal myeloid progenitor cells. One of the key mechanisms by which cell growth and proliferation are regulated involves the mitogenic signal transduction pathway. For example, cell growth is regulated, in part, through the cascade of mitogen-activated protein (MAP) kinase that also includes other transducing molecules such as MAP kinase kinase (MEK) and Raf-1. Constitutive activation of MAP kinases is associated with many cancer cell lines (e.g., pancreas, colon, lung, ovary, and kidney) and primary tumors from various human organs (e.g., kidney, colon, lung), and correlated with the simultaneous expression of MEK and Raf-1 (Hoshino, et al., Oncogene. 18(3):813-22 (1999)). Thus, the level and duration of MAP kinase expression thus appears to control these differential effects. Id.

One family of MAPKs, the p38 MAPK protein kinase family is activated primarily by cellular stresses and not mitogenic stimuli. The activation domain of p38 contains the sequence TGY, which represent the tyrosine and threonine residues required for activation (targeted by MKK3 and MKK6). The physiological role of the different p38 isoforms (which are derived from three genes as well as differential splicing) is still unclear. Among the identified targets for p38 are MAPKAPK-2 and the transcription factors, CHOP/GADD153 (Wang and Ron, Science (1997) 272, 1347-1349), MEF2C (Han et al., Nature (1997), 386, 296-299) and ATF2.

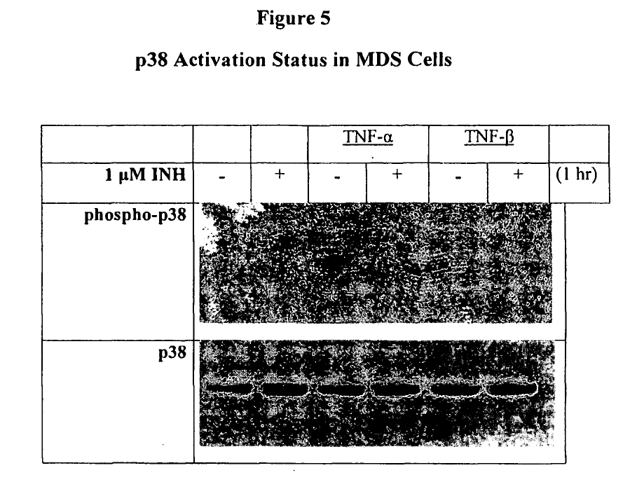

The activation of p38 MAP kinase (phosphorylated form) in MDS cells and in bone marrow stromal cells (BMSC) is induced by cytokines and other moieties present in the bone marrow milieu. (See FIG. 5). Activation of p38 MAP kinase may be induced even in normal myeloid progenitor cells by tumor necrosis factor (TNF). This activation may result in the secretion of cytokines thought to be involved in the pathogenesis of MDS. For example, secretion of certain cytokines is thought to play a role in making a bone marrow microenvironment that is hospitable to the growth and survival of MDS cells while make the bone marrow inhospitable for normal myeloid progenitor cells.

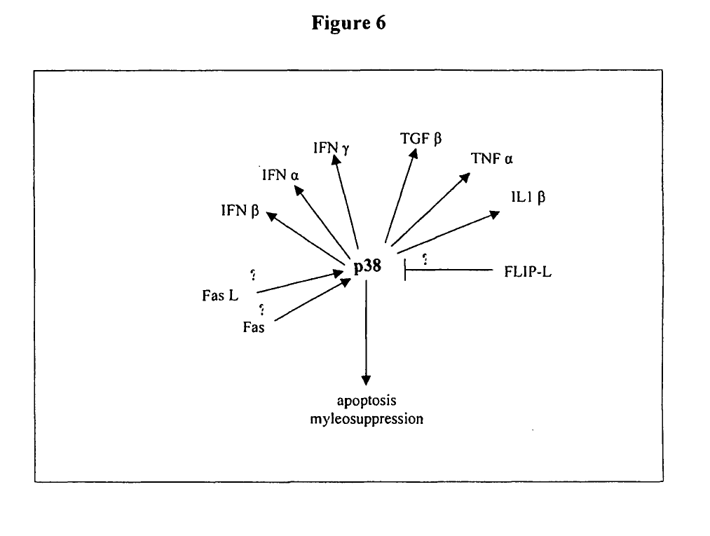

A number of cytokines are thought to play roles in the pathology of MDS. These cytokines include interleukin-1 (IL-1), interleukin-6 (IL-6), interleukin-11 (IL-11), tumor necrosis factor (TNF), insulin-like growth factor-1 (IGF-1), macrophage inflammatory protein-1 (MIP-1), receptor activator of NF-kappa B ligand (RANKL), and transforming growth factor-beta (TGF-β). (See FIG. 6.)

Administering MAP kinase inhibitors negatively impacts the bone marrow milieu in which MDS cells propagate by altering cytokine expression. For example, p38 inhibitors act to reduce interleukin-6 (IL-6) production from bone marrow stromal cells (BMSCs). Production of IL-6 is thought to be important for maintaining a microenvironment that is favorable for MDS cell proliferation, that is, MDS cell growth and replication. While the impact of p38 inhibitors on cytokine expression, such as IL-6 expression, is a likely mechanism by which to explain the therapeutic impact of p38 inhibitors on MDS, it is not the only mechanism available to explain these positive effects. Accordingly, this mechanism is provided solely as a tool for conceptualizing the role that p38 inhibitors can play in treating MDS and is not intended to be limiting in any way.

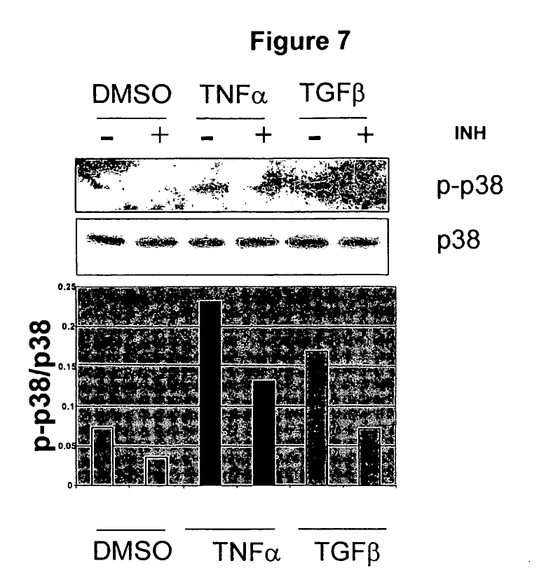

Tumor necrosis factors alpha and beta (TNF-α, TNF-β) are also considered cytokines that are upregulated in connection with MDS. Levels of p38 MAPK activity were shown to be increased in MDS cells when either TNF-α or TNF-β were provided. This upregulation is inhibited by the addition of the p38 MAPK inhibitor. (See FIG. 7). MDS cells are pre-treated for 1 hour with vehicle (−) or 1.0 μM of a p38 MAPK inhibitor (+) and then induced with either 1 ng/ml TNFα or 5 ng/ml TGFβ for 30 min. Phosphorylated p38 MAPK (p-p38) and total p38 levels are analyzed by Western blotting. The bar graph represents p-p38 levels relative to total p38 in each sample. In companion studies, the addition of the p38 MAPK inhibitor to MDS cells did not inhibit proliferation of the MDS cells nor did it induce cytotoxicity, as determined by cell viability assays.

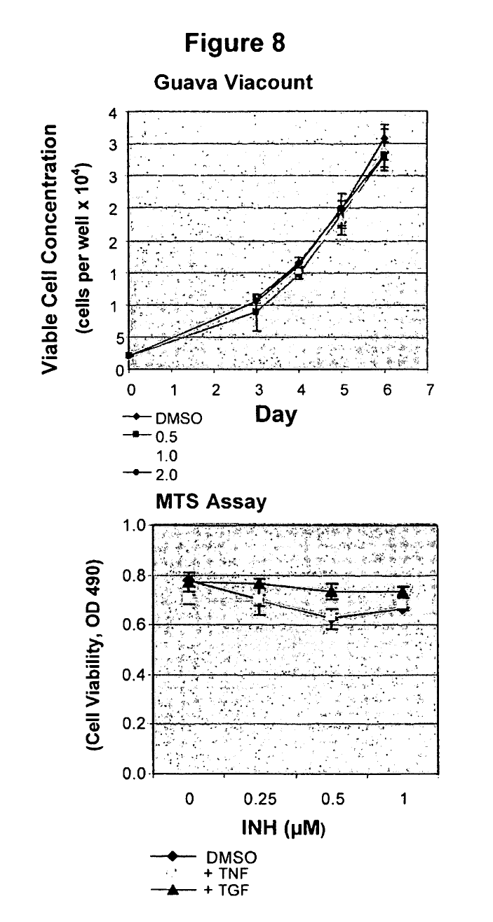

Culturing experiments that examined various combinations of bone marrow stromal cells (BMSC), bone marrow mononuclear cells (BMMNC) and MDS cells shows that TNF-α secretion from BMMNCs is induced by the presence of MDS cells. The induction of the TNF-α is inhibited by the addition of a p38 MAPK inhibitor. (See FIG. 8). In FIG. 8A, MDS cells (2,500 cells/well) are incubated with vehicle or with increasing concentrations of the p38 MAPK inhibitor and assayed for viability on different days within a 6-day period using the GUAVA VIACOUNT. In FIG. 8B, MDS cells (30,000 cells/well) are incubated with vehicle or with 1 ng/ml TNFα or 5 ng/ml TGFβ for 30 minutes in the absence or presence of increasing concentrations of the p38 MAPK inhibitor. Cell metabolic activity is measured after 72 hours using MTS. Each point represents the average of triplicate samples±SD.

Interestingly, TNF-α secretion from BMMNC is induced by the presence of MDS cells in a contact dependent manner. This induction of TNF-α secretion is inhibited by administration of a p38 MAPK inhibitor. TNF-α secretion from BMMNCs is suppressed by BMSC in a contact independent manner and again, TNF-α secretion is inhibited by addition of a p38 MAPK inhibitor.

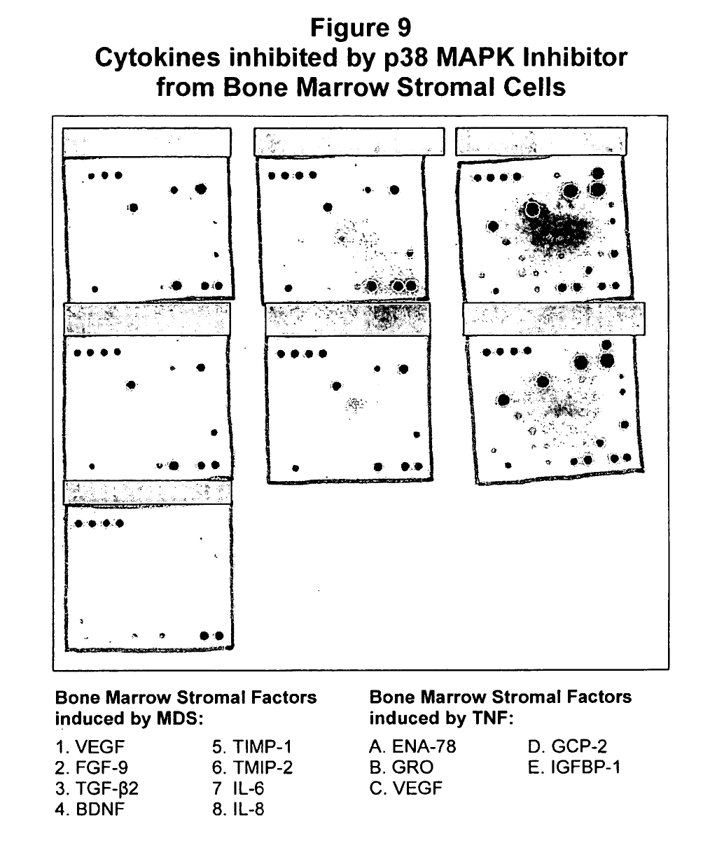

A cytokine array analysis of cytokines produced from bone marrow stromal cells in the presence of MDS cells is performed. Cytokines production from BMSCs induced by MDS cells includes vascular endothelial growth factor (VEGF), fibroblast growth factor 9 (FGF-9), Transforming Growth Factor beta-2 (TGF-β2) and brain-derived neurotrophic factor (BDNF). A separate cytokine array analysis is performed to identify cytokines produced from bone marrow stromal cells in the presence of TNF. Bone marrow stromal factors induced by TNF include epithelial neutrophil activating peptide-78 (ENA-78), which is an activator of neutrophils and is a member of the IL-8 subgroup of C-X-C family of chemokines, growth regulated oncogene (GRO), VEGF, granulocyte chemotactic peptide-2 (GCP-2), and insulin-like growth factor binding protein 1 (IGFBP-1). (See FIG. 9).

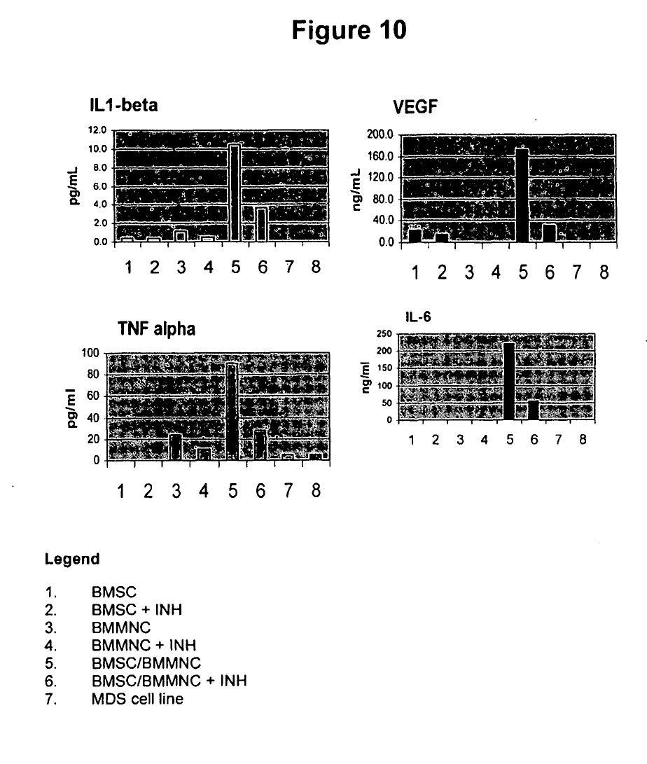

Administration of p38 MAPK inhibitors reduce the secretion from bone marrow cell culture of pro-inflammatory cytokines which are known to suppress hematopoiesis and which are elevated in low risk MDS bone marrow. BMSC and BMMNC, either alone or in co-culture, are incubated in the presence or absence of 0.5 μM of a p38 MAPK inhibitor for 5 days. Supernatants are collected, concentrated, and analyzed by SEARCHLIGHT CYTOKINES ARRAY TECHNOLOGY (PIERCE). Concentrations in conditioned media are calculated. As seen in FIG. 10, levels of IL-1β, VEGF, TNF-α, and IL-6 are produced at elevated levels by BMSCs in the presence of BMMNC are reduced when a p38 MAPK inhibitor is added to the culture. These results indicate that p38 MAPK inhibitors are effective to inhibit the production of immunosuppressive cytokines.

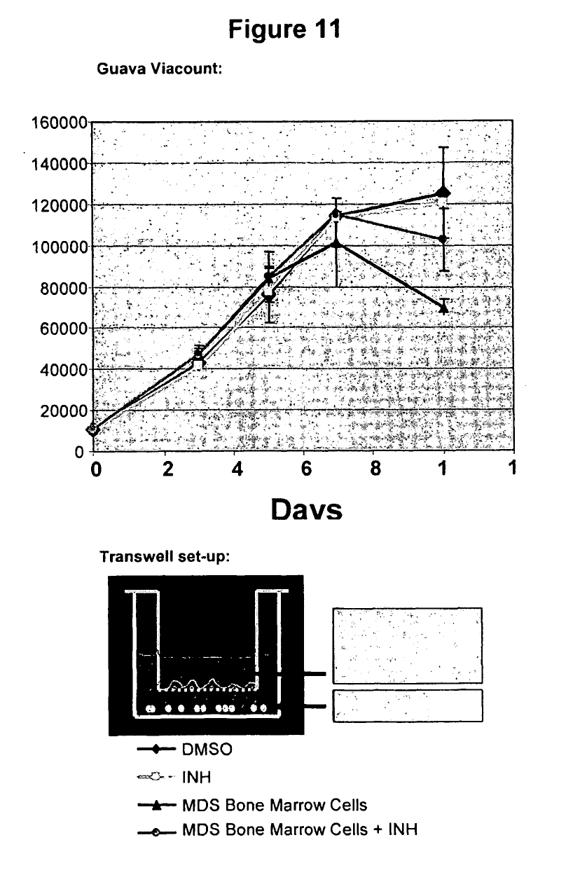

Another important effect p38 MAPK inhibitors have on MDS bone marrow is that they promote the proliferation of CD34+ hematopoietic progenitor cells. As seen in FIG. 11, the inhibition of proliferation of CD34+ hematopoietic progenitor cells by incubation with normal bone marrow cells in the present of a MDS cell line is suppressed by the presence of a p38 MAPK inhibitor.

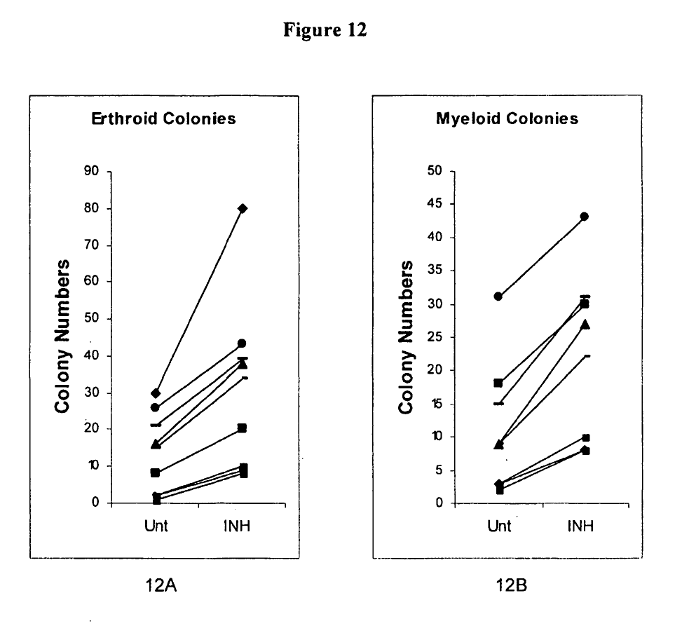

In related work, inhibition of p38 MAPK leads to increased erythroid and myeloid colony formation in MDS cultures. (See FIG. 12A). These results indicate that inhibition of p38 MAPK can serve as an effective treatment of MDS by allowing hematopoiesis in affected individuals to return to a more normal state.

The effectiveness of such a treatment is shown in FIG. 12A-B. The data shown in these figures illustrate that inhibition of p38 MAPK leads to an increase in burst forming unit erythroids (BFU-E) and colony forming unit granulocyte macrophages (CFU-GM). Burst forming unit erythroid (BFU-E) are the earliest known erythroid precursor cells that eventually differentiate into erythrocytes and are known to be CD33+ and CD34+. A reduced production of or a complete absence of BFU-E colonies is observed in patients with MDS. Colony forming unit granulocyte macrophage describe pluripotent precursor cells involved in hematopoiesis.

FIGS. 12B and 12C show that BFU-E and CFU-GM from MDS patients increases in number in the presence of increasing concentrations of p38 MAPK inhibitors.

In a set of related experiments, MDS bone marrow CD34+ cells are transfected with recombinant constructs expressing siRNA molecules directed against p38 MAPK. In these experiments, mean colony numbers of BFU-E and CFU-GM are dramatically increased in comparison to MDS bone marrow CD34+ cells transfected with a control siRNA construct. These data demonstrate that it is the act of inhibiting p38 MAPK activity generally, and not merely by providing a particular kinase inhibitor, which leads to the amelioration of the ineffective hematopoiesis characteristic of MDS.

Inhibitors of p38 MAP Kinase

As used herein, the term “inhibitor” includes, but is not limited to, any suitable molecule, compound, protein or fragment thereof, nucleic acid, formulation or substance that can regulate p38 MAP kinase activity. The data discussed herein can be reproduced using any disclosed p38 MAPK inhibitor. The inhibitor can affect a single p38 MAP kinase isoform (e.g., p38α, p38β, p38γ or p38δ), more than one isoform, or all isoforms of p38 MAP kinase. In a preferred embodiment, the inhibitor regulates the α isoform of p38 MAP kinase.

In a preferred embodiment of the disclosed invention, it is contemplated that the particular inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

If acting on p38 MAP kinase directly, in one embodiment the inhibitor should exhibit an IC₅₀ value of about 5 μM or less, preferably about 500 nM or less, more preferably about 100 nM or less. In a related embodiment, the inhibitor should exhibit an IC₅₀ value relative to the p38α MAP kinase isoform that is about ten fold less than that observed when the same inhibitor is tested against other p38 MAP kinase isoforms in a comparable assay.

Those skilled in the art can determine whether or not a compound is useful in the disclosed invention by evaluating its p38 MAP kinase activity as well as its relative IC₅₀ value. This evaluation can be accomplished through conventional in vitro assays. In vitro assays include assays that assess inhibition of kinase or ATPase activity of activated p38 MAP kinase. In vitro assays can also assess the ability of the inhibitor to bind to a p38 MAP kinase or to reduce or block an identified downstream effect of the activated p38 MAP kinase, e.g., cytokine secretion. IC₅₀ values are calculated using the concentration of inhibitor that causes a 50% decrease as compared to a control.

A binding assay is a fairly inexpensive and simple in vitro assay to run. As previously mentioned, binding of a molecule to p38 MAP kinase, in and of itself, can be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 MAP kinase regulatory activity.

The target in a binding assay can be either free in solution, fixed to a support, or expressed in or on the surface of a cell. A label (e.g., radioactive, fluorescent, quenching, etc.) can be placed on the target, compound, or both to determine the presence or absence of binding. This approach can also be used to conduct a competitive binding assay to assess the inhibition of binding of a target to a natural or artificial substrate or binding partner. In any case, one can measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor can be encoded by a nucleic acid that is introduced into the cell wherein the cell produces the potential inhibitor itself.

Alternative assays involving in vitro analysis of potential inhibitors include those where cells (e.g., HeLa) transfected with DNA coding for relevant kinases can be activated with substances such as sorbitol, IL-1, TNF, or PMA. After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are terminated by the addition of SDS loading buffer. Phosphorylated substrate is resolved through SDS-PAGE and visualized and quantitated in a phosphorimager. The p38 MAP kinase regulation, in terms of phosphorylation and IC₅₀ values, can be determined by quantitation. See e.g., Kumar, S. et al., Biochem. Biophys. Res. Commun. 235:533-538 (1997). Similar techniques can be used to evaluate the effects of potential inhibitors on other MAP kinases.

Other in vitro assays can assess the production of TNF-α as a correlation to p38 MAP kinase activity. One such example is a Human Whole Blood Assay. In this assay, venous blood is collected from, e.g., healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific Co., San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath, and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an Enriched Mononuclear Cell Assay. The enriched mononuclear cell assay begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

Exemplary Inhibitors

Preferred examples of the compounds of the invention are of the formula:

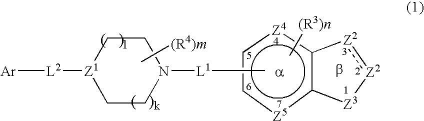

and the pharmaceutically acceptable salts thereof, or a pharmaceutical composition thereof, wherein 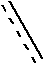
represents a single or double bond;

one Z² is CA or CR⁸A and the other is CR¹, CR¹₂, NR⁶ or N wherein each R¹, R⁶ and R⁸ is independently hydrogen or noninterfering substituent;

A is —Wᵢ—COXⱼY wherein Y is COR² or an isostere thereof and R² is hydrogen or a noninterfering substituent, each of W and X is a spacer of 2-6 Å, and each of i and j is independently 0 or 1;

Z³ is NR⁷ or O;

each of Z⁴ and Z⁵ is independently N or CR¹ wherein R¹ is as defined above and wherein at least one of Z⁴ and Z⁵ is N;

each R³ is independently a noninterfering substituent;

n is 0-3;

each of L¹ and L² is a linker;

each R⁴ is independently a noninterfering substituent;

m is 0-4;

Z¹ is CR⁵ or N wherein R⁵ is hydrogen or a noninterfering substituent;

each of l and k is an integer from 0-2 wherein the sum of l and k is 0-3;

Ar is an aryl group substituted with 0-5 noninterfering substituents, wherein two noninterfering substituents can form a fused ring.

Preferred embodiments of compounds useful in the invention are derivatives of indole-type compounds containing a mandatory substituent, A, at a position corresponding to the 2- or 3-position of indole. In general, an indole-type nucleus is preferred, although alternatives within the scope of the invention are also illustrated below. Additionally, PCT publication WO00/71535, published 7 Dec. 2000, discloses indole derived compounds that are specific inhibitors of p38 kinase. The disclosure of this document is incorporated herein by reference.

U.S. Provisional Patent Application No. 60/417,599 filed 9 Oct. 2002 and U.S. patent application Ser. No. 10/683,656, filed Oct. 9, 2003, disclose azaindole derivatives that are useful in treating conditions that are characterized by enhanced p38 activity and are therefore useful for purposes of this invention. The disclosure of these documents is incorporated herein by reference.

As used herein, a “noninterfering substituent” is a substituent which either leaves the ability of the compound of formula (1) to inhibit p38-α activity qualitatively intact or enhances the activity of the inhibitor. Thus, the substituent may alter the degree of inhibition of p38. However, as long as the compound of formula (1) retains the ability to inhibit p38 activity, the substituent will be classified as “noninterfering.” As mentioned above, a number of assays for determining the ability of any compound to inhibit p38 activity are available in the art. A whole blood assay for this evaluation is illustrated below: the gene for p38 has been cloned and the protein can be prepared recombinantly and its activity assessed, including an assessment of the ability of an arbitrarily chosen compound to interfere with this activity. The essential features of the molecule are tightly defined. The positions which are occupied by “noninterfering substituents” can be substituted by conventional organic moieties as is understood in the art. It is irrelevant to the present invention to test the outer limits of such substitutions.

Regarding the compounds of formula (1), L¹ and L² are described herein as linkers. The nature of such linkers is typically less important that the distance they impart between the portions of the molecule. Typical linkers include alkylene, i.e. (CH₂)ₙ—R; alkenylene—i.e., an alkylene moiety which contains a double bond, including a double bond at one terminus. Other suitable linkers include, for example, substituted alkylenes or alkenylenes, carbonyl moieties, and the like.

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, “inorganic residue” refers to a residue that does not contain carbon. Examples include, but are not limited to, halo, hydroxy, NO₂ or NH₂.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

When the compounds of Formula 1 contain one, or more chiral centers, the invention includes optically pure forms as well as mixtures of stereoisomers or enantiomers.

With respect to the portion of the compound of formula (1) between the atom of Ar bound to L² and ring α, L¹ and L² are linkers which space the substituent Ar from ring α at a distance of 4.5-24 Å, preferably 6-20 Å, more preferably 7.5-10 Å. In a preferred embodiment, the distance of substituent Ar from ring is less than 24 Å. The distance is measured from the center of the a ring to the atom of Ar to which the linker L² is attached. Typical, but nonlimiting, embodiments of L¹ and L² are CO and isosteres thereof, or optionally substituted isosteres, or longer chain forms. L², in particular, may be alkylene or alkenylene optionally substituted with noninterfering substituents or L¹ or L² may be or may include a heteroatom such as N, S or O. Such substituents include, but are limited to, a moiety selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety.

Isosteres of CO and CH₂, include SO, SO₂, or CHOH. CO and CH₂ are preferred.

Thus, L² is substituted with 0-2 substituents. Where appropriate, two optional substituents on L² can be joined to form a non-aromatic saturated or unsaturated hydrocarbyl ring that includes 0-3 heteroatoms such as O, S and/or N and which contains 3 to 8 members. Two optional substituents on L² can be joined to form a carbonyl moiety which can be subsequently converted to an oxime, an oximeether, an oximeester, or a ketal.

Ar is aryl, heteroaryl, including 6-5 fused heteroaryl, cycloaliphatic or cycloheteroaliphatic that can be optionally substituted. Ar is preferably optionally substituted phenyl.

Each substituent on Ar is independently a hydrocarbyl residue (1-20C) containing 0-5 heteroatoms selected from O, S and N, or is an inorganic residue. Preferred substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members. More preferred substituents include halo, alkyl (1-4C) and more preferably, fluoro, chloro and methyl. These substituents may occupy all available positions of the aryl ring of Ar, preferably 1-2 positions, most preferably one position. These substituents may be optionally substituted with substituents similar to those listed. Of course some substituents, such as halo, are not further substituted, as known to one skilled in the art.

Two substituents on Ar can be joined to form a fused, optionally substituted aromatic or nonaromatic, saturated or unsaturated ring which contains 3-8 members.

Regarding formula (1), between L¹ and L² is a piperidine-type moiety of the following formula:

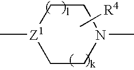

Z¹ is CR⁵ or N wherein R⁵ is H or a noninterfering substituent. Each of l and k is an integer from 0-2 wherein the sum of l and k is 0-3. The noninterfering substituents R⁵ include, without limitation, halo, alkyl, alkoxy, aryl, arylalkyl, aryloxy, heteroaryl, acyl, carboxy, or hydroxy. Preferably, R⁵ is H, alkyl, OR, NR₂, SR or halo, where R is H or alkyl. Additionally, R⁵ can be joined with an R⁴ substituent to form an optionally substituted non-aromatic saturated or unsaturated hydrocarbyl ring which contains 3-8 members and 0-3 heteroatoms such as O, N and/or S. Preferred embodiments include compounds wherein Z¹ is CH or N, and those wherein both l and k are 1.

R⁴ represents a noninterfering substituent such as a hydrocarbyl residue (1-20C) containing 0-5 heteroatoms selected from O, S and N. Preferably R⁴ is alkyl, alkoxy, aryl, arylalkyl, aryloxy, heteroalkyl, heteroaryl, heteroarylalkyl, RCO, ═O, acyl, halo, CN, OR, NRCOR, NR, wherein R is H, alkyl (preferably 1-4C), aryl, or hetero forms thereof. Each appropriate substituent is itself unsubstituted or substituted with 1-3 substituents. The substituents are preferably independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof. R⁴ may occur m times on the ring; m is an integer of 0-4. Preferred embodiments of R⁴ comprise alkyl (1-4C) especially two alkyl substituents and carbonyl. Most preferably R⁴ comprises two methyl groups at positions 2 and 5 or 3 and 6 of a piperidinyl or piperazinyl ring or ═O preferably at the 5-position of the ring. The substituted forms may be chiral and an isolated enantiomer may be preferred.

R³ also represents a noninterfering substituent. Such substituents include hydrocarbyl residues (1-6C) containing 0-2 heteroatoms selected from O, S and/or N and inorganic residues. n is an integer of 0-3, preferably 0 or 1. Preferably, the substituents represented by R³ are independently halo, alkyl, heteroalkyl, OCOR, OR, NRCOR, SR, or NR₂, wherein R is H, alkyl, aryl, or heteroforms thereof. More preferably R³ substituents are selected from alkyl, alkoxy or halo, and most preferably methoxy, methyl, and chloro. Most preferably, n is 0 and the α ring is unsubstituted, except for L¹ or n is 1 and R³ is halo or methoxy.

In the ring labeled β, Z³ may be NR₇ or O— i.e., the compounds may be related to indole or benzofuran. If C³ is NR⁷, preferred embodiments of R⁷ include H or optionally substituted alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof. More preferably, R⁷ is hydrogen or is alkyl (1-4C), preferably methyl or is acyl (1-4C), or is COOR wherein R is H, alkyl, alkenyl of aryl or hetero forms thereof. R⁷ is also preferably a substituted alkyl wherein the preferred substituents are form ether linkages or contain sulfinic or sulfonic acid moieties. Other preferred substituents include sulfhydryl substituted alkyl substituents. Still other preferred substituents include CONR₂ wherein R is defined as above.

It is preferred that the indicated dotted line represents a double bond; however, compounds which contain a saturated β ring are also included within the scope of the invention.

Preferably, the mandatory substituent CA or CR⁸A is in the 3-position; regardless of which position this substituent occupies, the other position is CR¹, CR¹₂, NR₆ or N. CR¹ is preferred. Preferred embodiments of R¹ include hydrogen, alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R¹ can be joined to form a fused, optionally substituted aromatic or nonaromatic, saturated or unsaturated ring which contains 3-8 members. Most preferably, R¹ is H, alkyl, such as methyl, most preferably, the ring labeled a contains a double bond and CR¹ is CH or C-alkyl. Other preferable forms of R¹ include H, alkyl, acyl, aryl, arylalkyl, heteroalkyl, heteroaryl, halo, OR, NR₂, SR, NRCOR, alkyl-OOR, RCO, COOR, and CN, wherein each R is independently H, alkyl, or aryl or heteroforms thereof.

While the position not occupied by CA is preferred to include CR¹, the position can also be N or NR₆. While NR₆ is less preferred (as in that case the ring labeled P would be saturated), if NR₆ is present, preferred embodiments of R⁶ include H, or alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, or R₃Si wherein each R is independently H, alkyl, alkenyl or aryl or heteroforms thereof.

Preferably, CR⁸A or CA occupy position 3- and preferably Z² in that position is CA. However, if the β ring is saturated and R⁸ is present, preferred embodiments for R⁸ include H, halo, alkyl, alkenyl and the like. Preferably R⁸ is a relatively small substituent corresponding, for example, to H or lower alkyl 1-4C.

A is —Wᵢ—COXⱼY wherein Y is COR² or an isostere thereof and R² is a noninterfering substituent. Each of W and X is a spacer and may be, for example, optionally substituted alkyl, alkenyl, or alkynyl, each of i and j is 0 or 1. Preferably, W and X are unsubstituted. Preferably, j is 0 so that the two carbonyl groups are adjacent to each other. Preferably, also, i is 0 so that the proximal CO is adjacent the ring. However, compounds wherein the proximal CO is spaced from the ring can readily be prepared by selective reduction of an initially glyoxal substituted β ring. In the most preferred embodiments of the invention, the α/β ring system is an indole containing CA in position 3- and wherein A is COCR².

The noninterfering substituent represented by R², when R² is other than H, is a hydrocarbyl residue (1-20C) containing 0-5 heteroatoms selected from O, S and/or N or is an inorganic residue. Preferred are embodiments wherein R² is H, or is straight or branched chain alkyl, alkenyl, alkynyl, aryl, arylalkyl, heteroalkyl, heteroaryl, or heteroarylalkyl, each optionally substituted with halo, alkyl, heteroalkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroatom-containing forms thereof, or wherein R² is OR, NR₂, SR, NRCONR₂, OCONR₂, or NRSO₂NR₂, wherein each R is independently H, alkyl, alkenyl or aryl or the heteroatom-containing forms thereof, and wherein two R attached to the same atom may form a 3-8 member ring and wherein said ring may further be substituted by alkyl, alkenyl, alkynyl, aryl, arylalkyl, heteroalkyl, heteroaryl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroatom-containing forms thereof wherein two R attached to the same atom may form a 3-8 member ring, optionally substituted as above defined.

Other preferred embodiments of R² are H, heteroarylalkyl, —NR₂, heteroaryl, —COOR, —NHRNR₂, heteroaryl-COOR, heteroaryloxy, —OR, heteroaryl-NR₂, —NROR and alkyl. Most preferably R² is isopropyl piperazinyl, methyl piperazinyl, dimethylamine, piperazinyl, isobutyl carboxylate, oxycarbonylethyl, morpholinyl, aminoethyldimethylamine, isobutyl carboxylate piperazinyl, oxypiperazinyl, ethylcarboxylate piperazinyl, methoxy, ethoxy, hydroxy, methyl, amine, aminoethyl pyrrolidinyl, aminopropanediol, piperidinyl, pyrrolidinyl-piperidinyl, or methyl piperidinyl.

Isosteres of COR² as represented by Y are defined as follows.

The isosteres have varying lipophilicity and may contribute to enhanced metabolic stability. Thus, Y, as shown, may be replaced by the isosteres in Table 1.

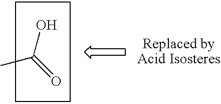

TABLE 1
Acid Isosteres

| Names | | |
|---|---|---|
| of Groups | Chemical Structures | Substitution Groups (SG) |
| tetrazole | 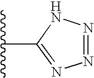 | n/a |
| 1,2,3-triazole | 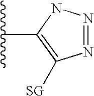 | H; SCH₃; COCH₃; Br; SOCH₃; SO₂CH₃; NO₂; CF₃; CN; COOMe |
| 1,2,4-triazole | 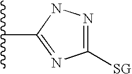 | H; SCH₃; COCH₃; Br; SOCH₃; SO₂CH₃; NO₂ |
| imidazole | 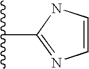 | H; SCH₃; COCH₃; Br; SOCH₃; SO₂CH₃; NO₂ |

Thus, isosteres include tetrazole, 1,2,3-triazole, 1,2,4-triazole and imidazole.

The compounds of formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Compounds useful in the practice of the disclosed invention include, but are not limited to, the compounds shown in Table 2, below.

TABLE 2
Exemplary p38 Inhibitors

| Cpd. # | Mol. Structure |
|---|---|
| 1 | 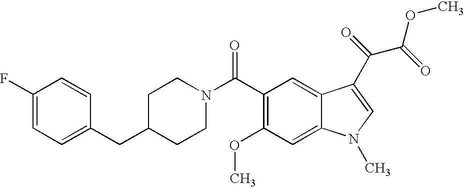 |
| 2 | 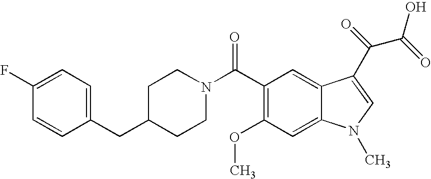 |
| 3 | 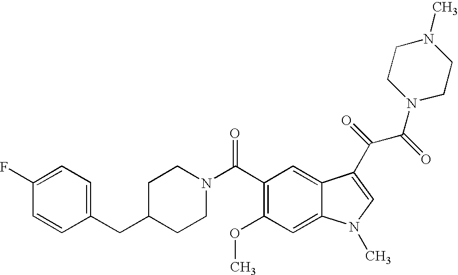 |
| 4 | 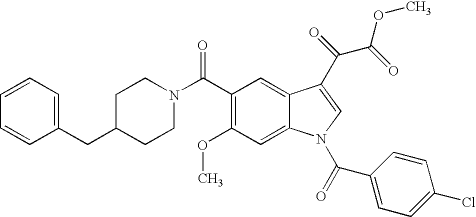 |
| 5 | 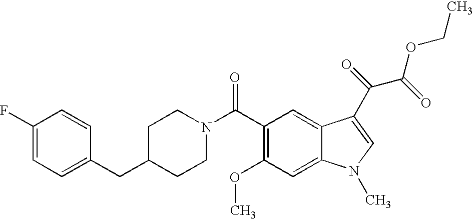 |
| 6 | 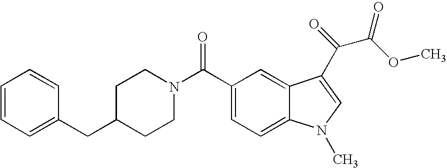 |
| 7 | 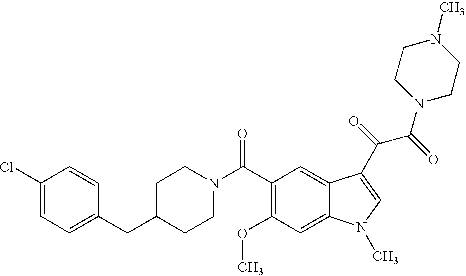 |
| 8 | 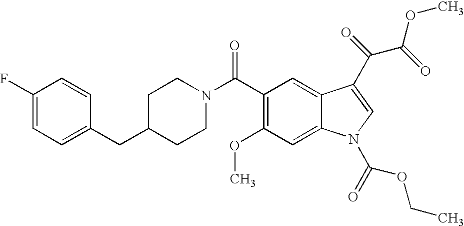 |
| 9 | 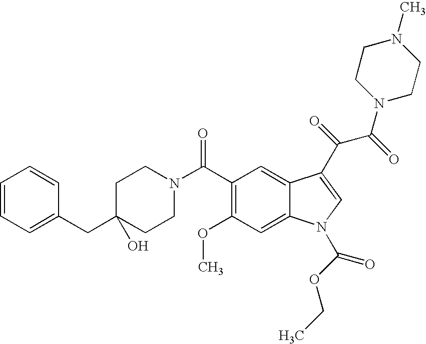 |
| 10 | 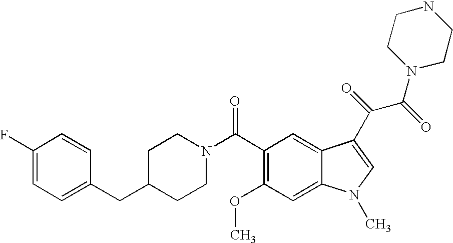 |
| 11 | 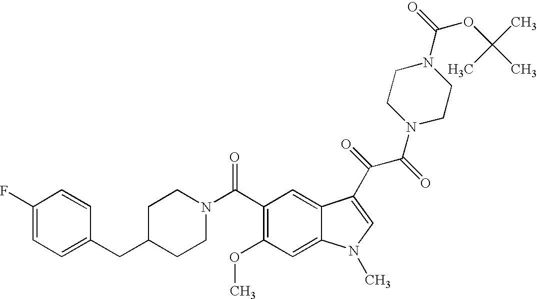 |
| 12 | 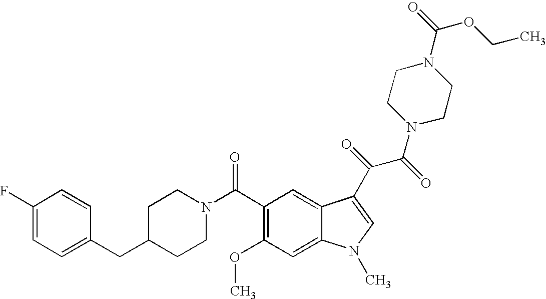 |
| 13 | 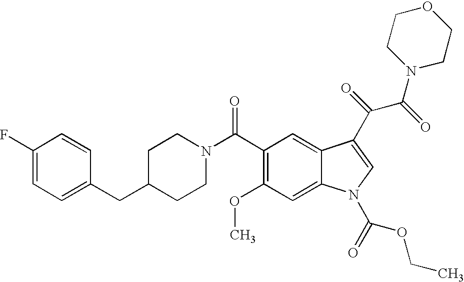 |
| 14 | 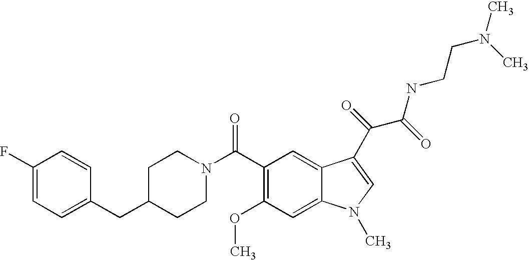 |
| 15 | 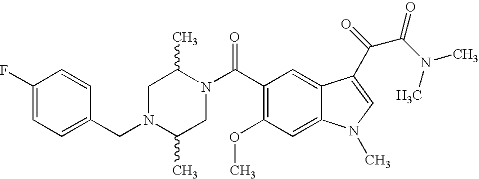 |
| 16 | 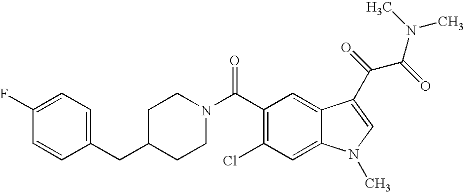 |
| 17 | 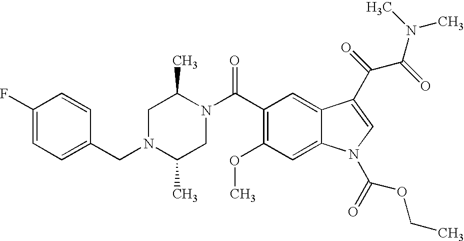 |
| 18 | 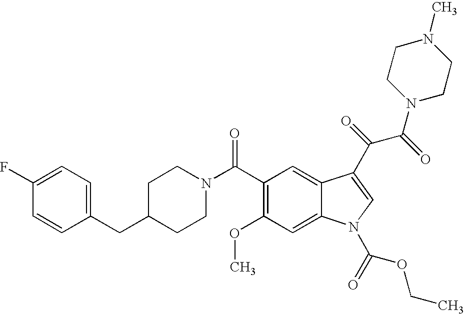 |
| 19 | 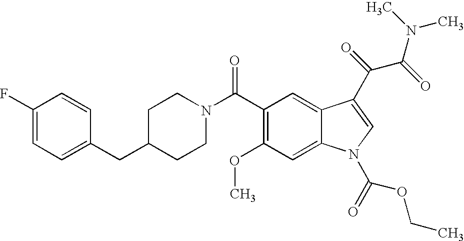 |
| 20 | 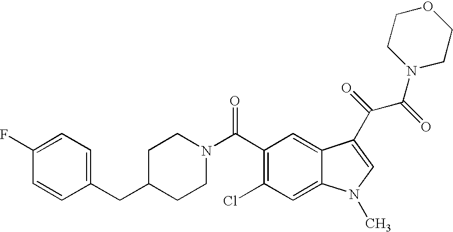 |
| 21 | 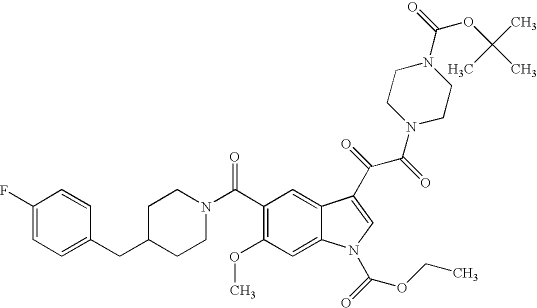 |
| 22 | 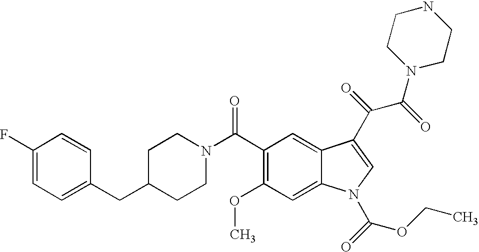 |
| 23 | 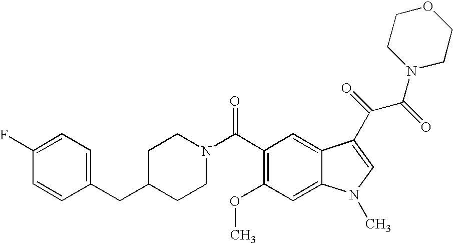 |
| 24 | 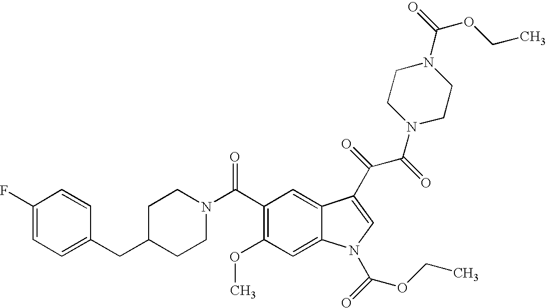 |
| 25 | 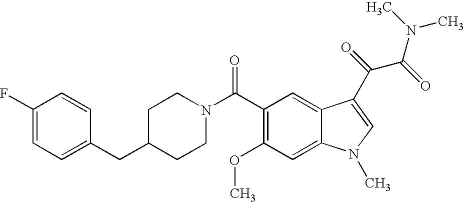 |
| 26 | 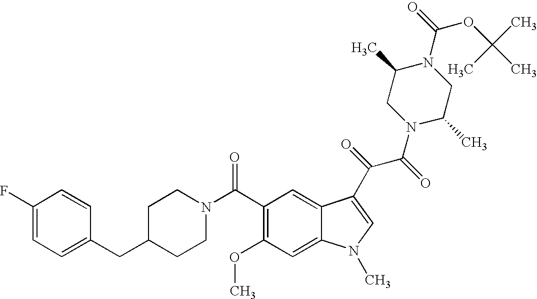 |
| 27 | 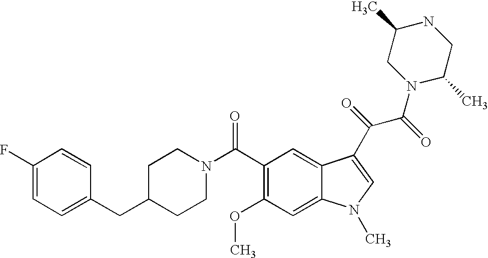 |
| 28 | 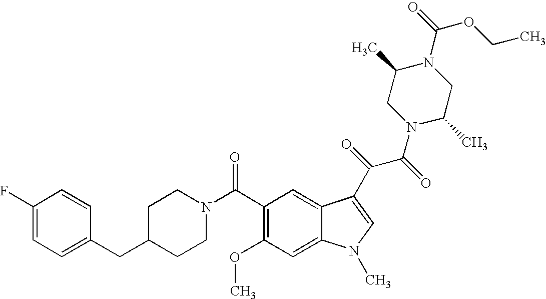 |
| 29 | 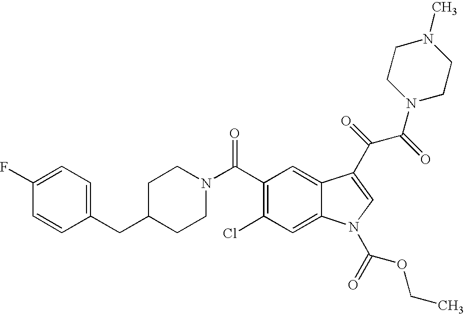 |
| 30 | 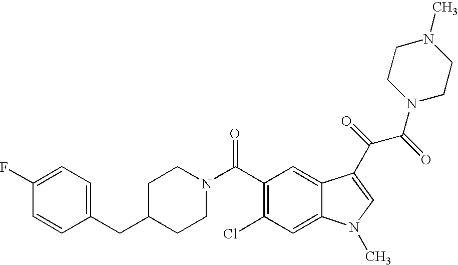 |
| 31 | 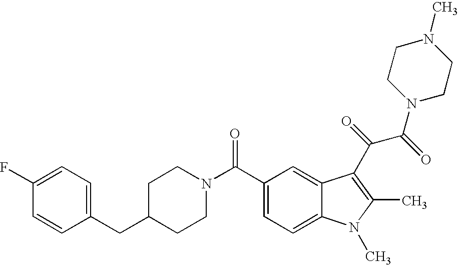 |
| 32 | 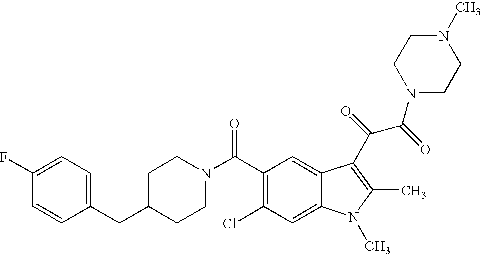 |
| 33 | 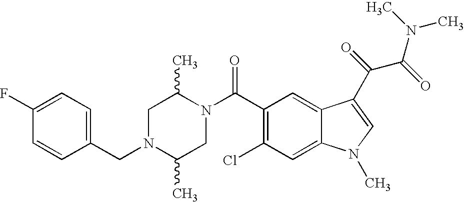 |
| 34 | 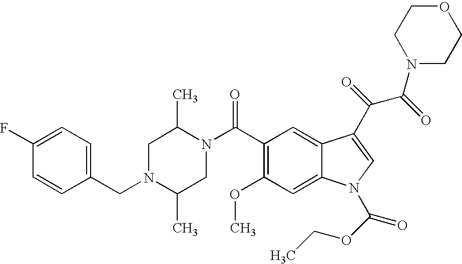 |
| 35 | 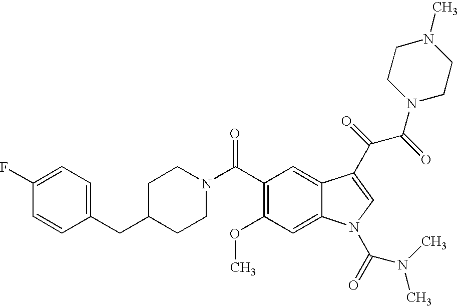 |
| 36 | 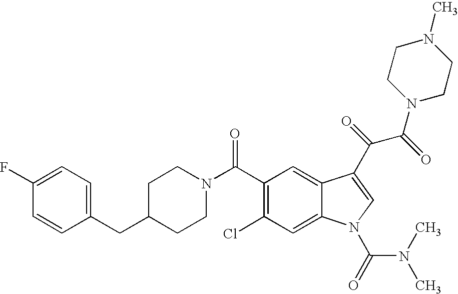 |
| 37 | 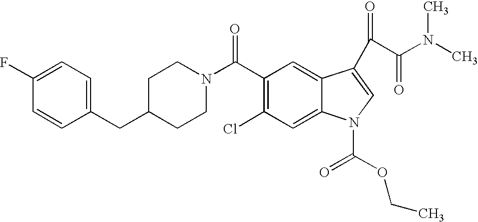 |
| 38 | 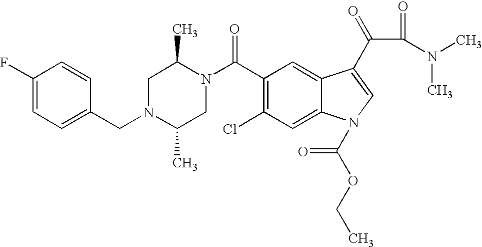 |
| 39 | 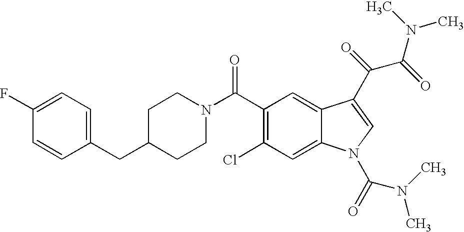 |
| 40 | 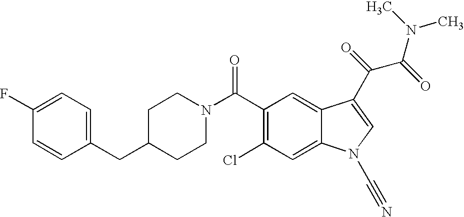 |
| 41 | 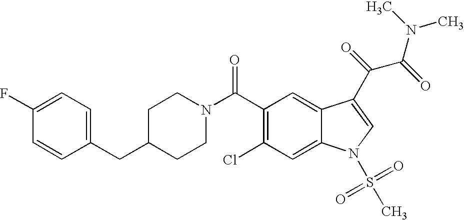 |
| 42 | 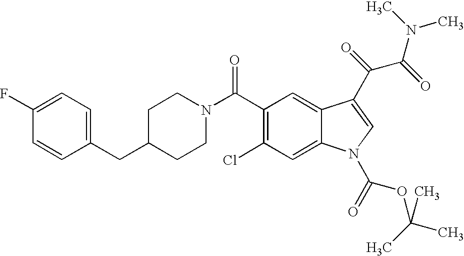 |
| 43 | 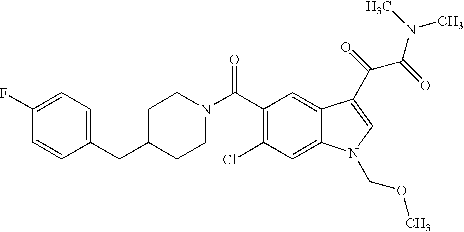 |
| 44 | 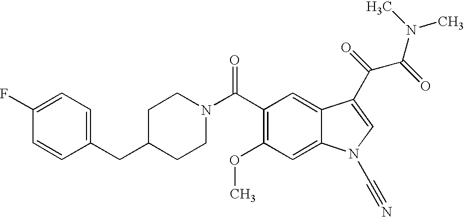 |
| 45 | 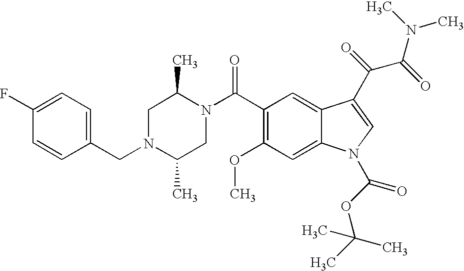 |
| 46 | 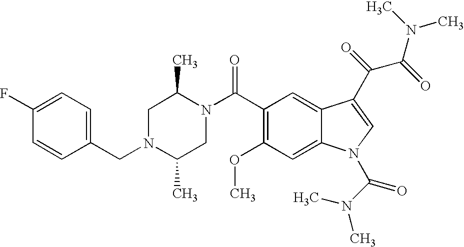 |
| 47 | 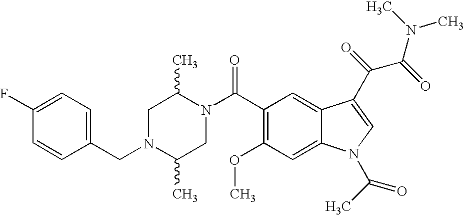 |
| 48 | 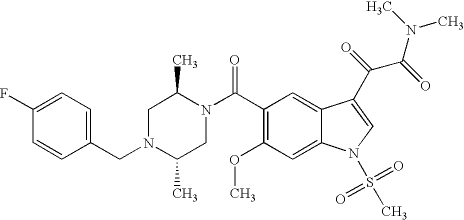 |
| 49 | 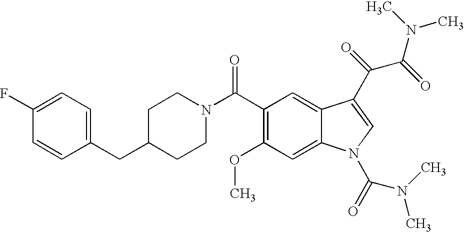 |
| 50 | 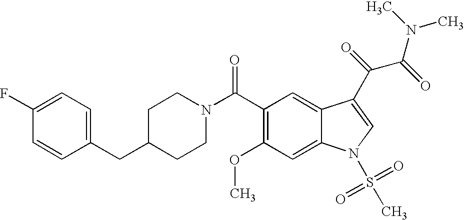 |
| 51 | 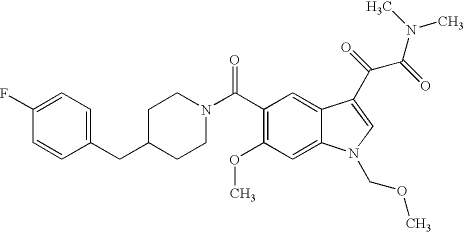 |
| 52 | 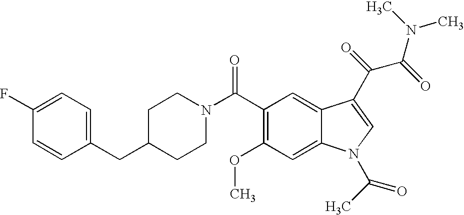 |
| 53 | 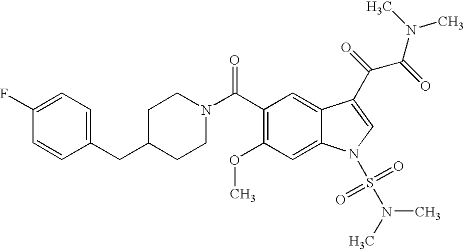 |
| 54 | 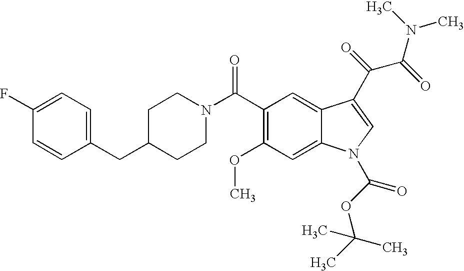 |
| 55 | 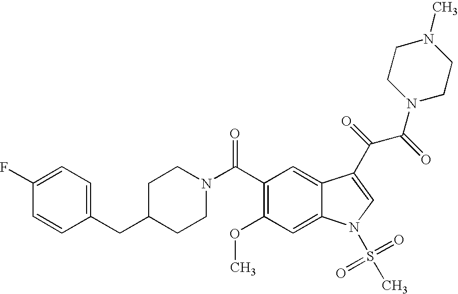 |
| 56 | 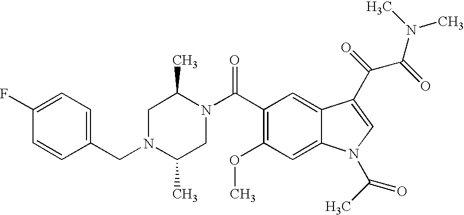 |
| 57 | 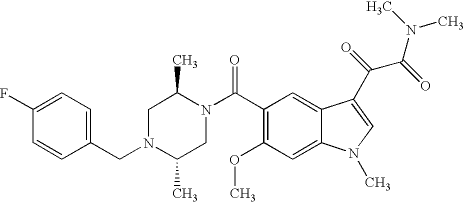 |
| 58 | 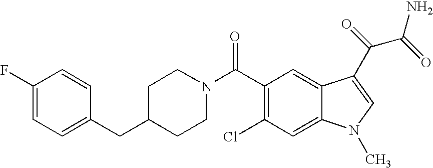 |
| 59 | 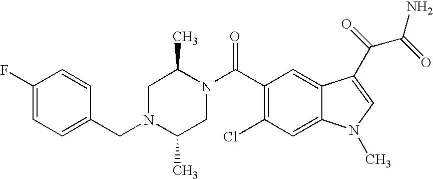 |
| 60 | 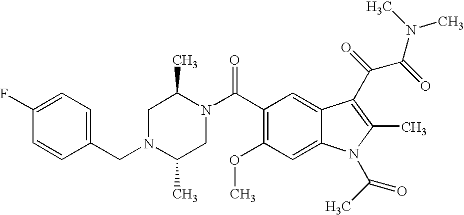 |
| 61 | 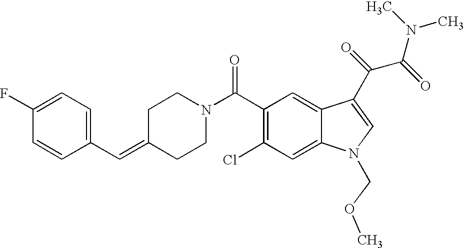 |
| 62 | 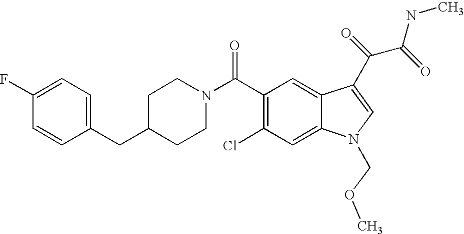 |
| 63 | 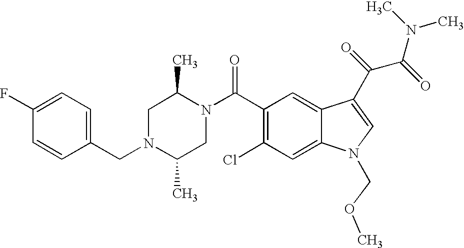 |
| 64 | 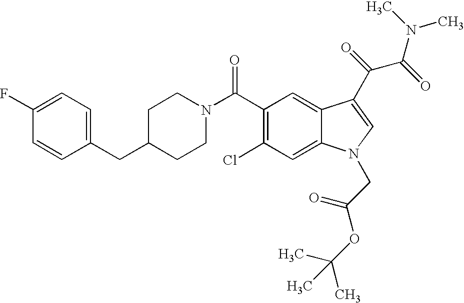 |
| 65 | 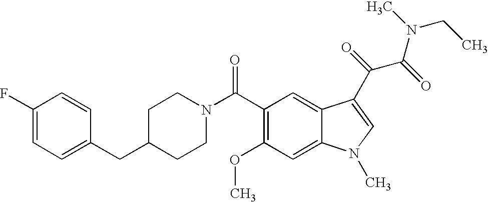 |
| 66 | 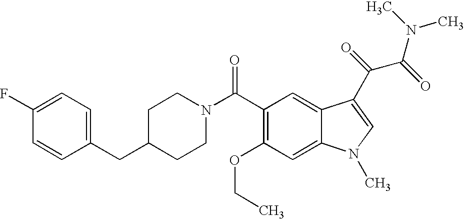 |
| 67 | 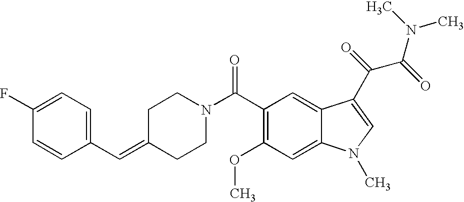 |
| 68 | 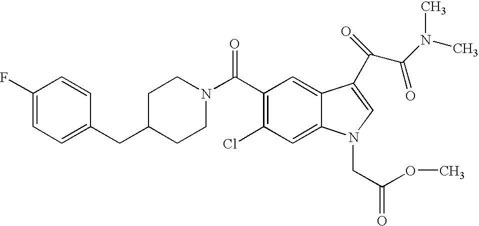 |
| 69 | 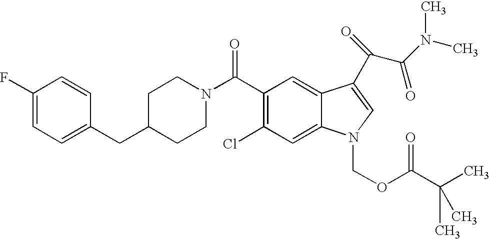 |
| 70 | 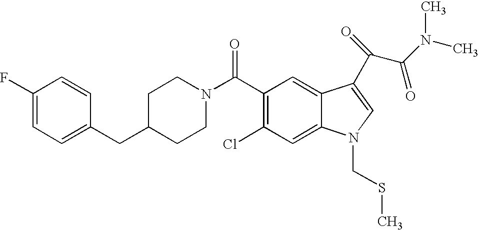 |
| 71 | 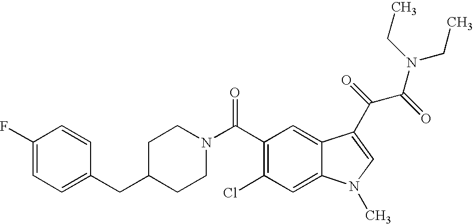 |
| 72 | 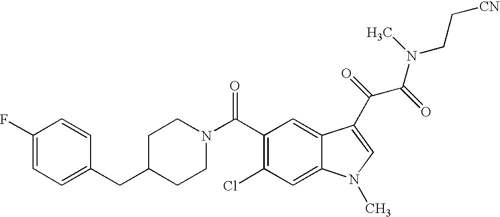 |
| 73 | 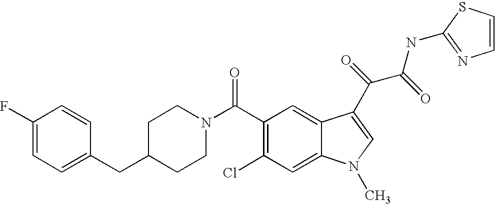 |
| 74 | 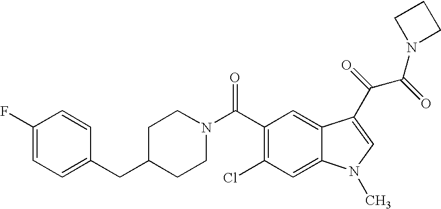 |
| 75 | 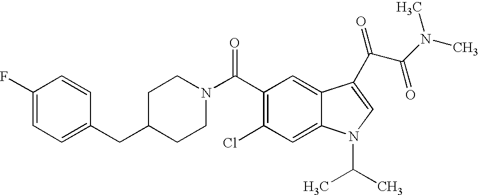 |
| 76 | 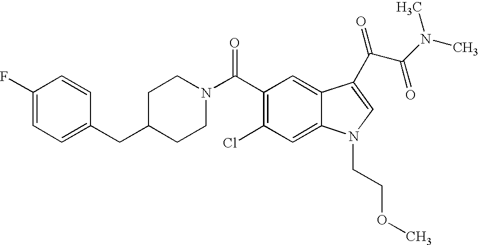 |
| 77 | 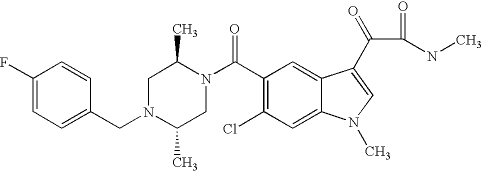 |
| 78 | 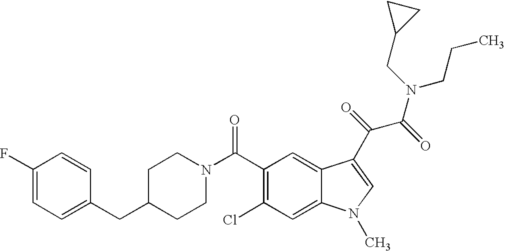 |
| 79 | 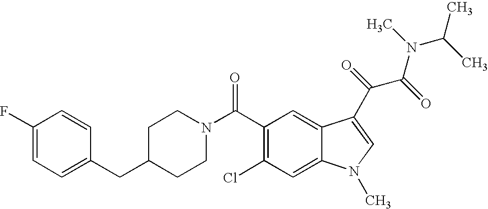 |
| 80 | 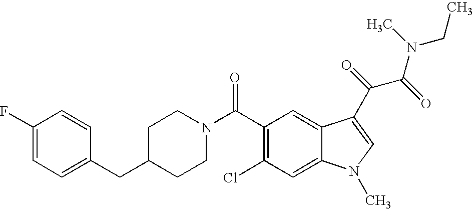 |
| 81 | 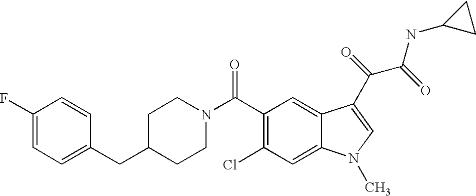 |
| 82 | 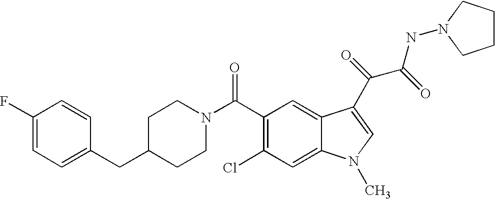 |
| 83 | 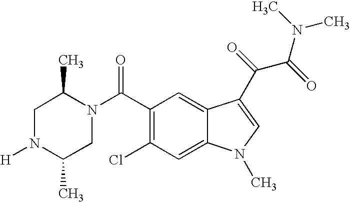 |
| 84 | 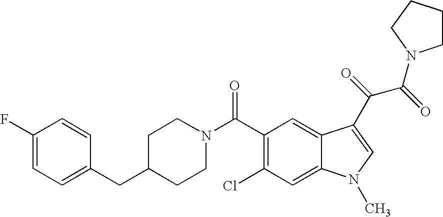 |
| 85 | 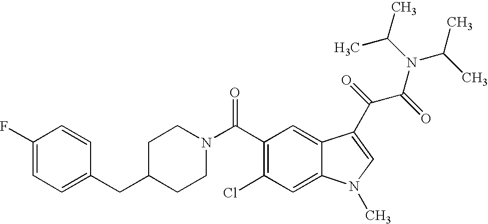 |
| 86 | 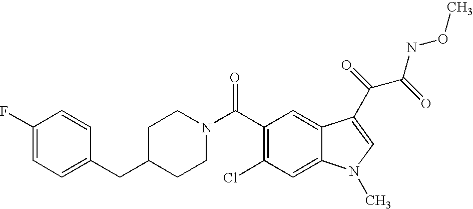 |
| 87 | 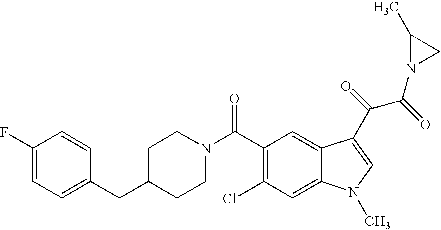 |
| 88 | 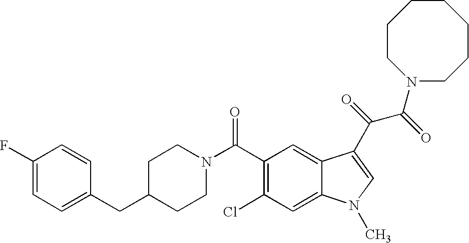 |
| 89 | 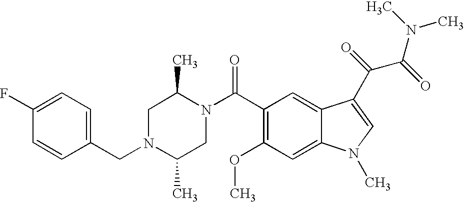 |
| 90 | 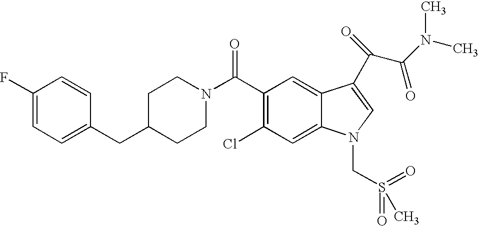 |
| 91 | 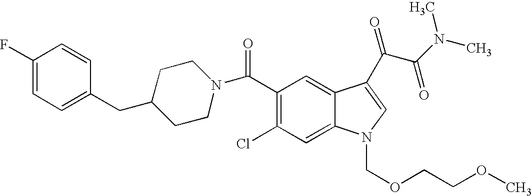 |
| 92 | 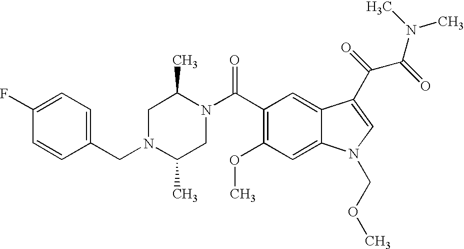 |
| 93 | 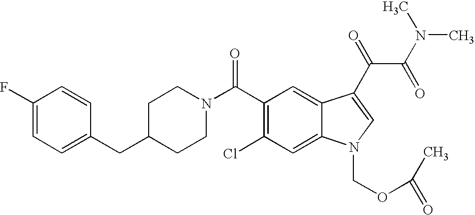 |
| 94 | 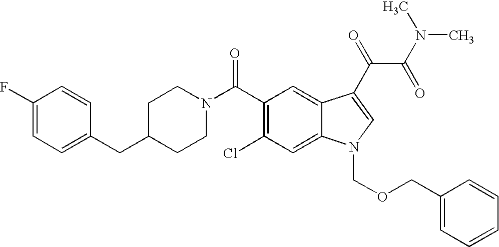 |
| 95 | 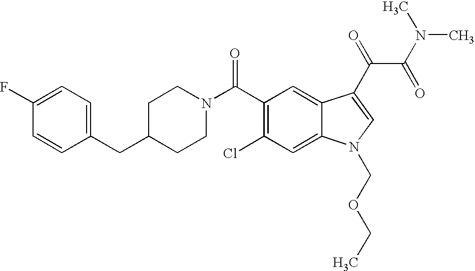 |
| 96 | 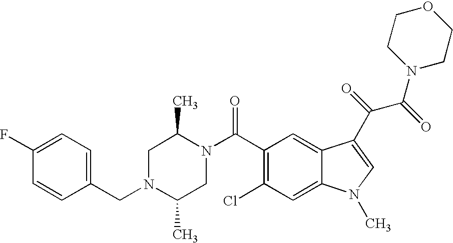 |
| 97 | 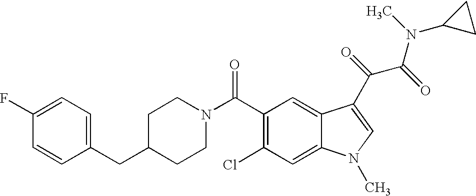 |
| 98 | 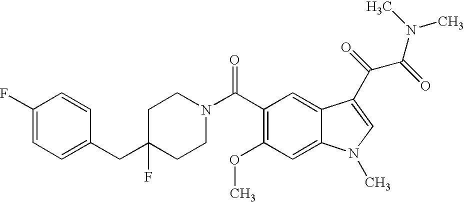 |
| 99 | 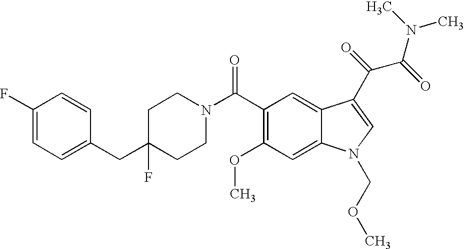 |
| 100 | 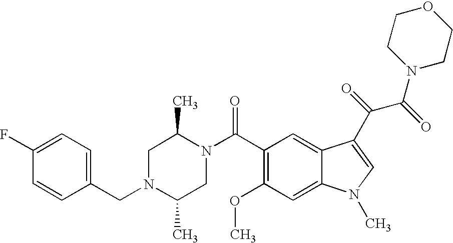 |
| 101 | 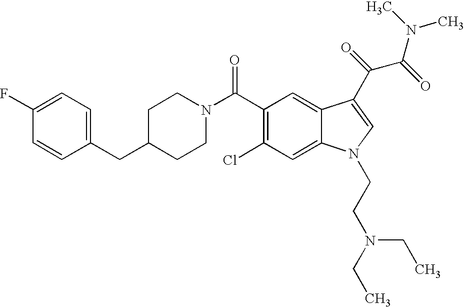 |
| 102 | 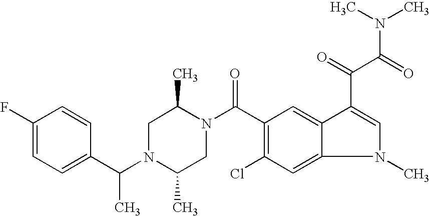 |
| 103 | 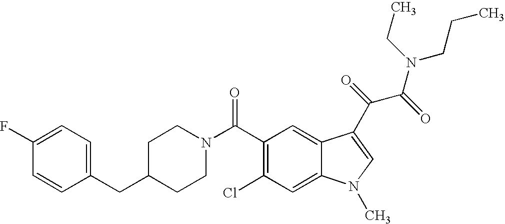 |
| 104 | 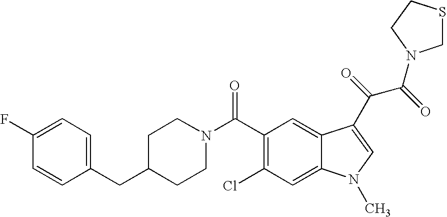 |
| 105 | 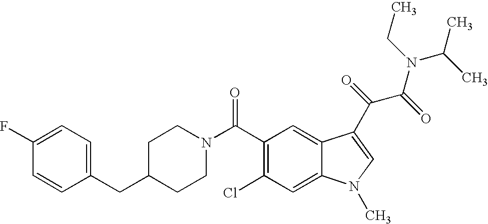 |
| 106 | 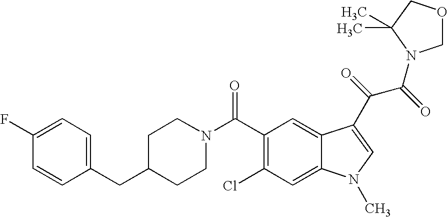 |
| 107 | 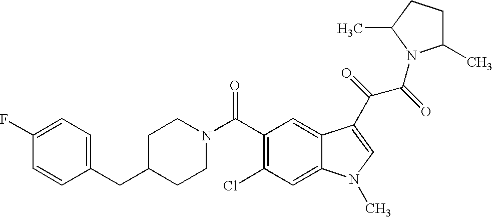 |
| 108 | 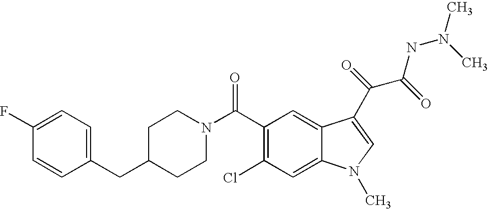 |
| 109 | 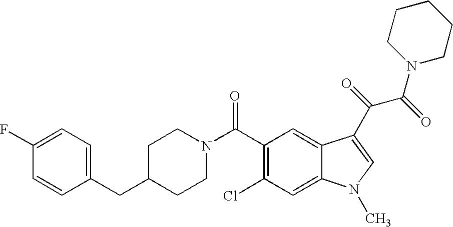 |
| 110 | 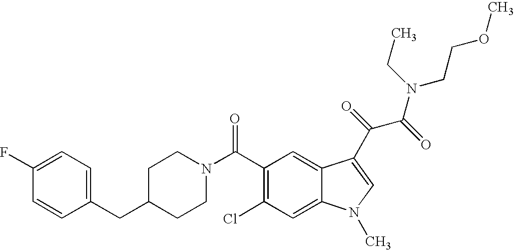 |
| 111 | 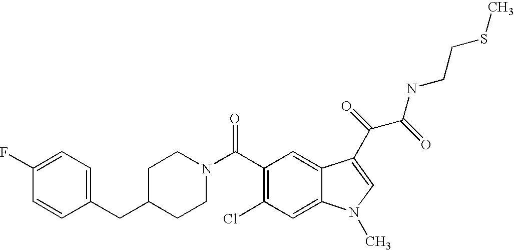 |
| 112 | 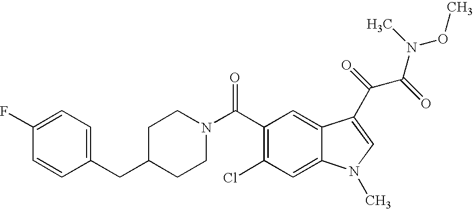 |
| 113 | 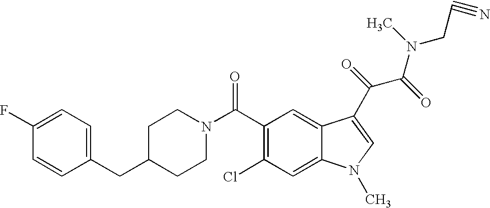 |
| 114 | 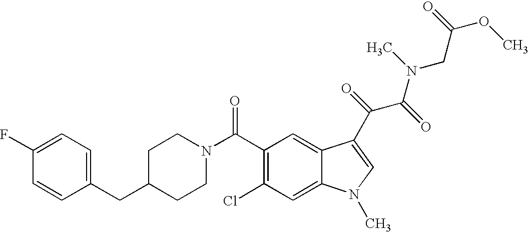 |
| 115 | 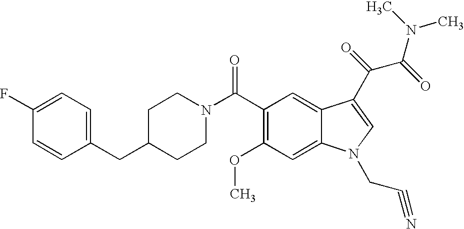 |
| 116 | 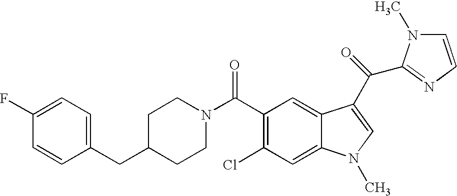 |
| 117 | 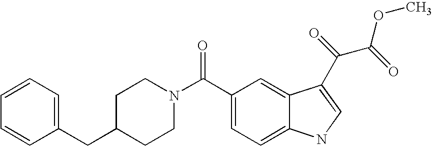 |
| 118 | 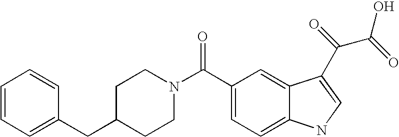 |
| 119 | 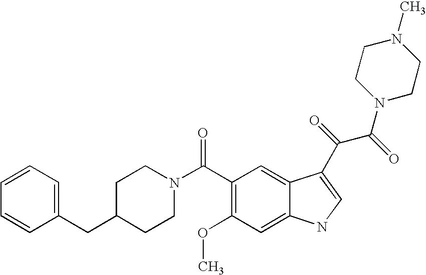 |
| 120 | 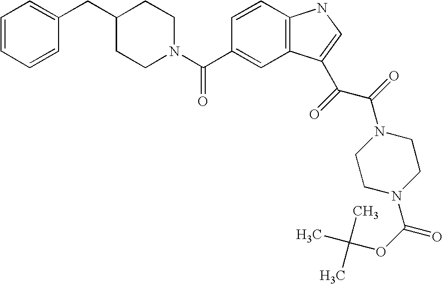 |
| 121 | 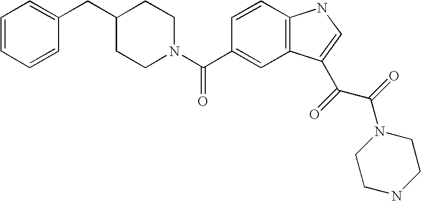 |
| 122 | 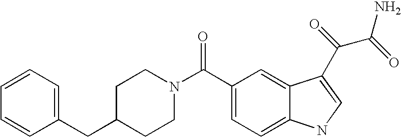 |
| 123 | 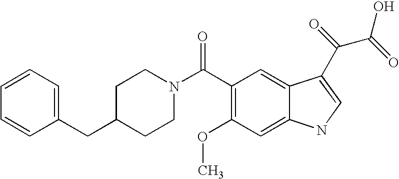 |
| 124 | 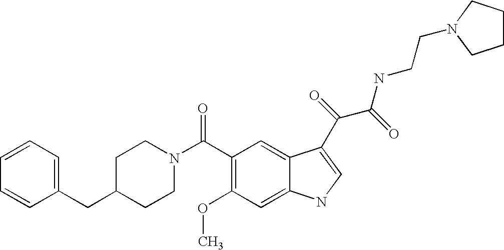 |
| 125 | 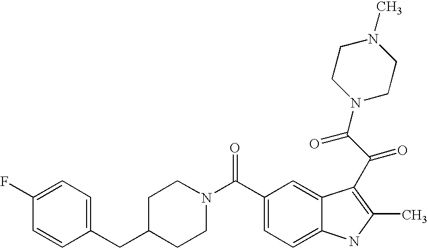 |
| 126 | 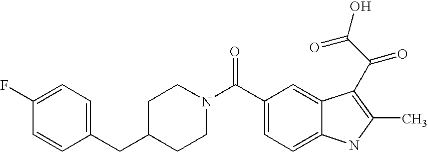 |
| 127 | 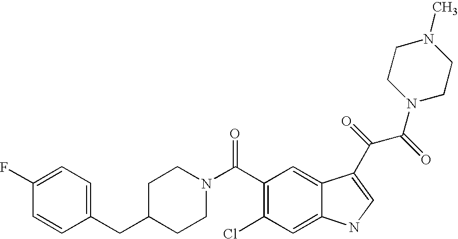 |
| 128 | 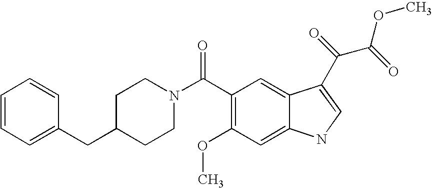 |
| 129 | 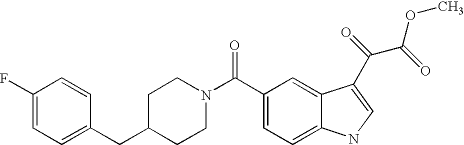 |
| 130 | 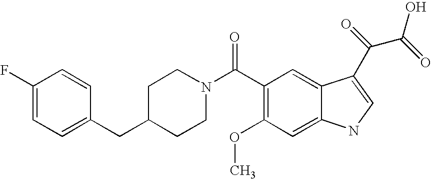 |
| 131 | 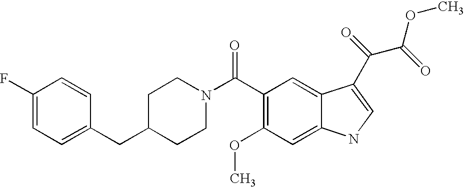 |
| 132 | 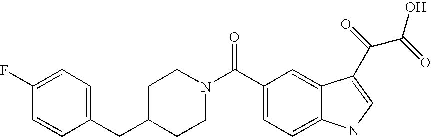 |
| 133 | 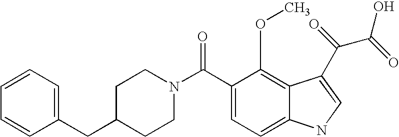 |
| 134 | 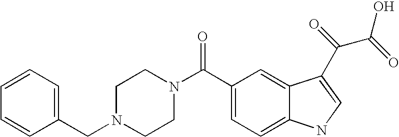 |
| 135 | 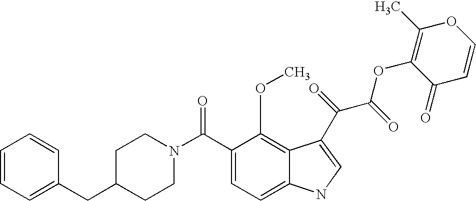 |
| 136 | 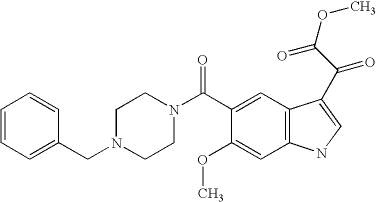 |
| 137 | 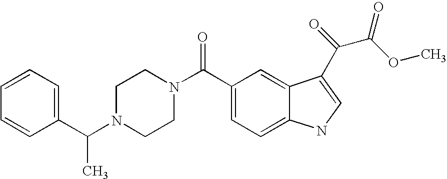 |
| 138 | 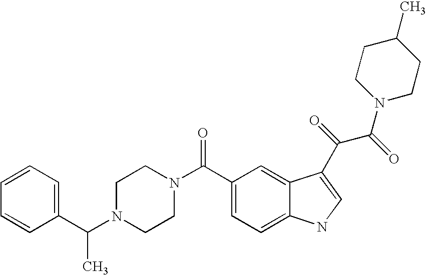 |
| 139 | 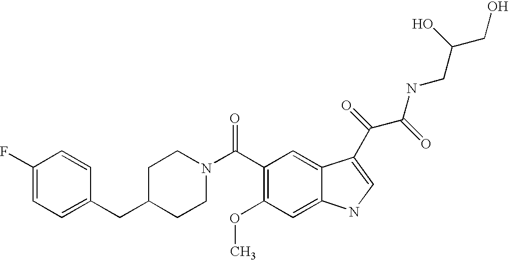 |
| 140 | 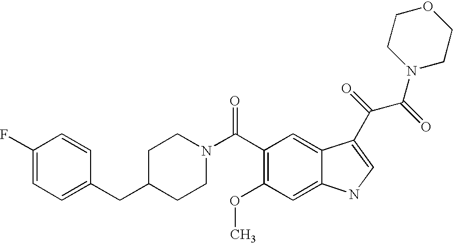 |
| 141 | 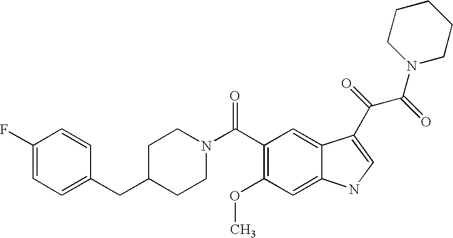 |
| 142 | 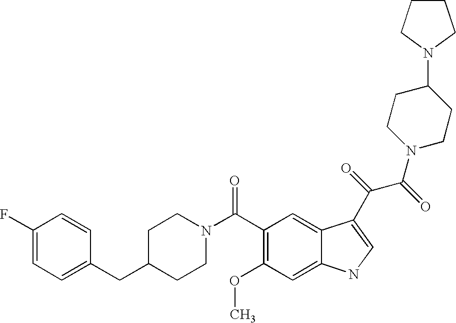 |
| 143 | 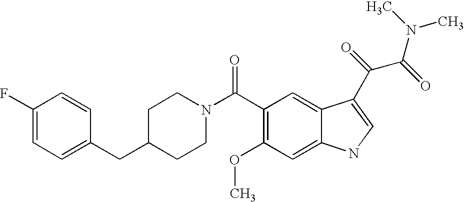 |
| 144 | 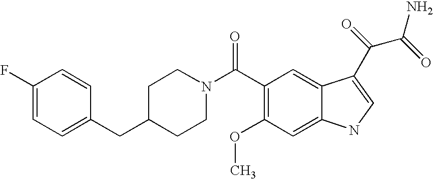 |
| 145 | 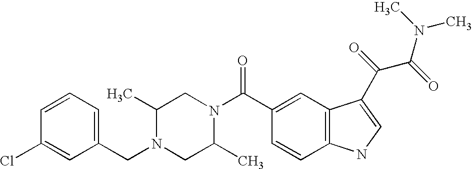 |
| 146 | 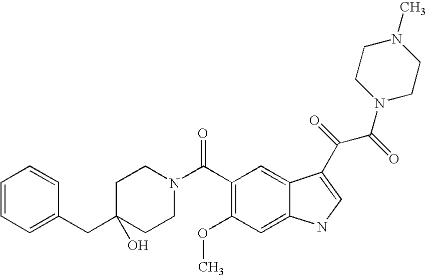 |
| 147 | 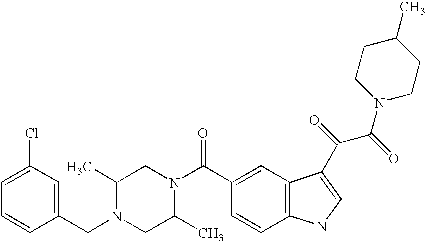 |
| 148 | 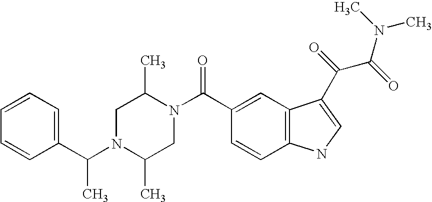 |
| 149 | 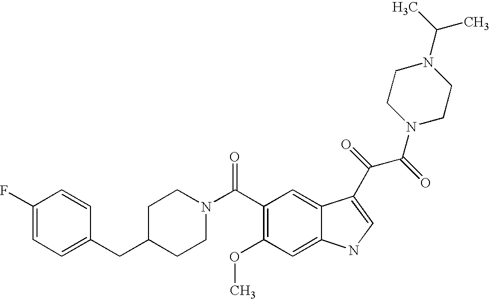 |
| 150 | 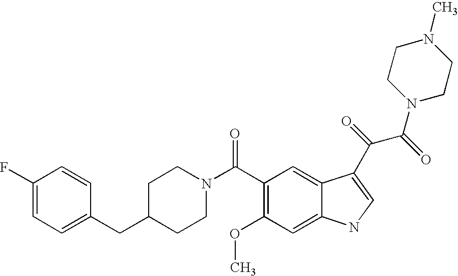 |
| 151 | 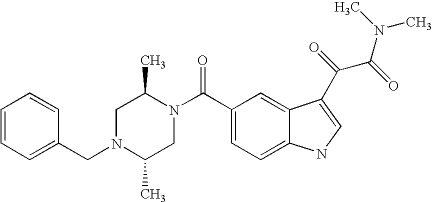 |
| 152 | 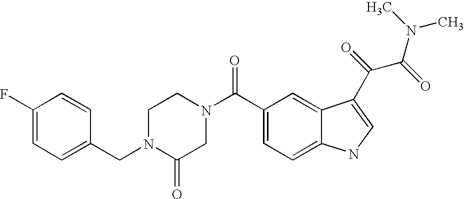 |
| 153 | 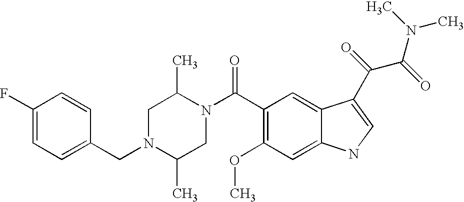 |
| 154 | 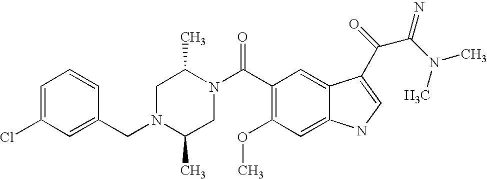 |
| 155 | 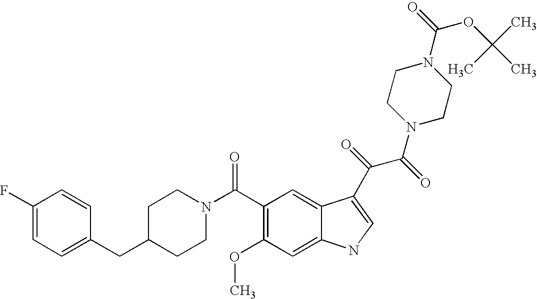 |
| 156 | 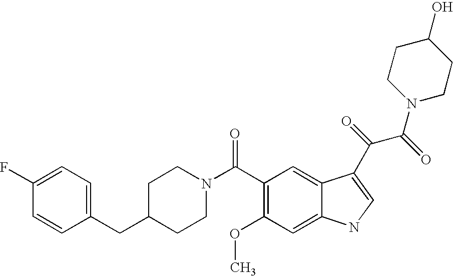 |
| 157 | 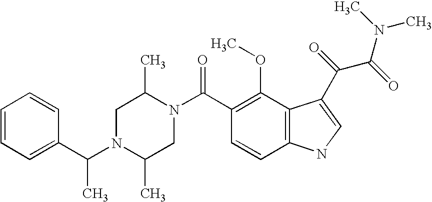 |
| 158 | 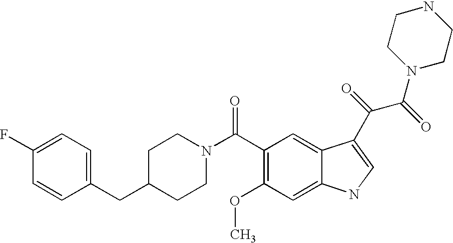 |
| 159 | 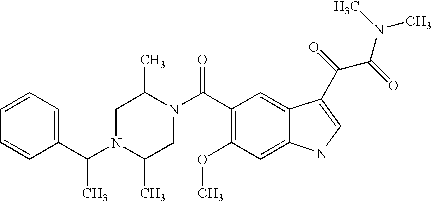 |
| 160 | 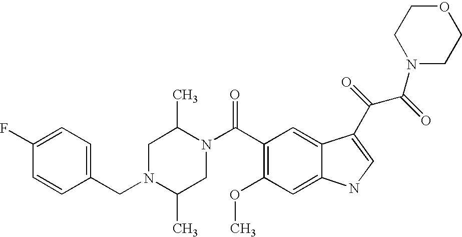 |
| 161 | 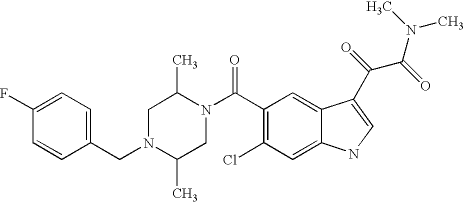 |
| 162 | 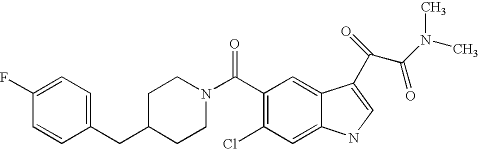 |
| 163 | 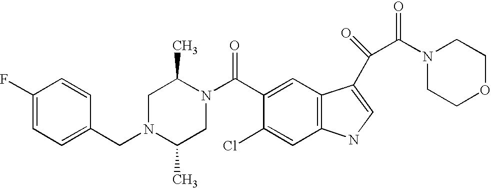 |
| 164 | 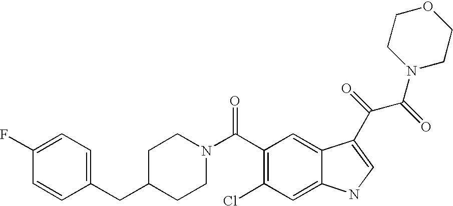 |
| 165 | 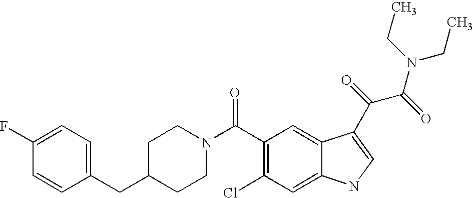 |
| 166 | 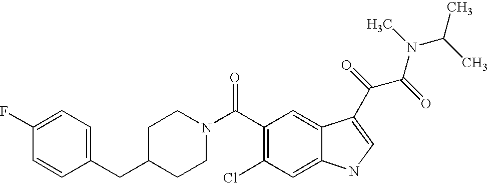 |
| 167 | 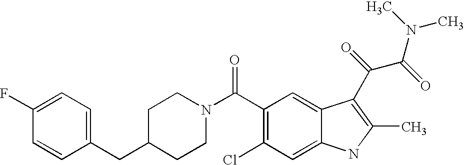 |
| 168 | 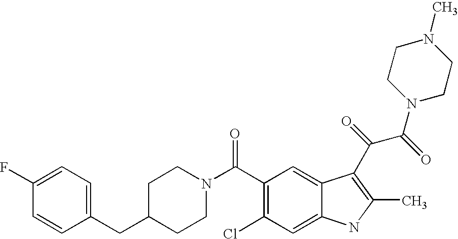 |
| 169 | 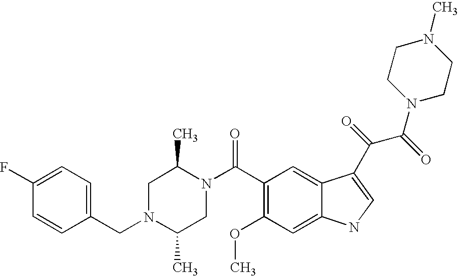 |
| 170 | 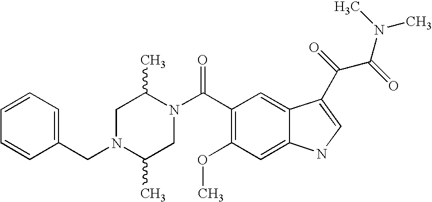 |
| 171 | 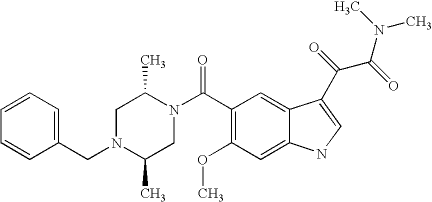 |
| 172 | 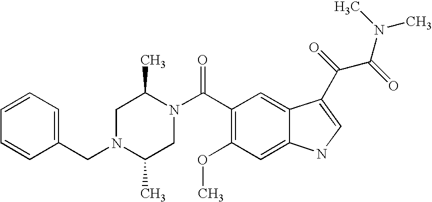 |
| 173 | 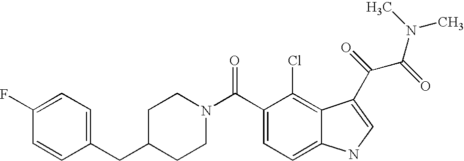 |
| 174 | 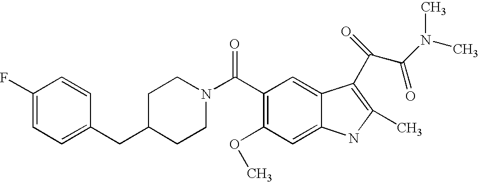 |
| 175 | 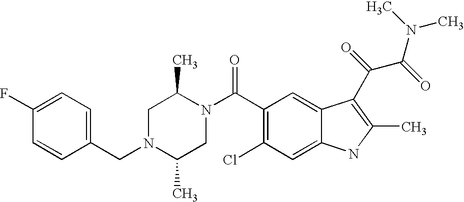 |
| 176 | 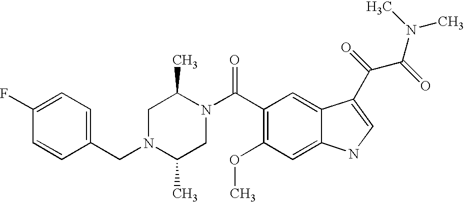 |
| 177 | 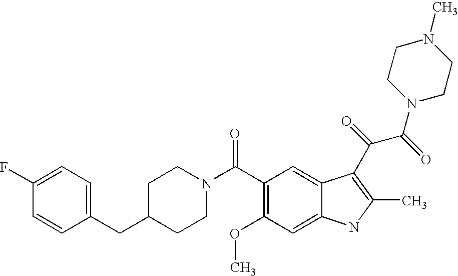 |
| 178 | 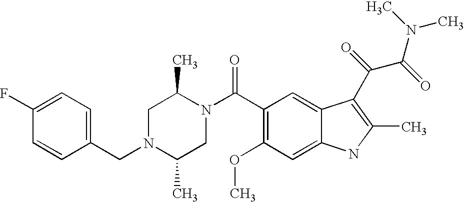 |
| 179 | 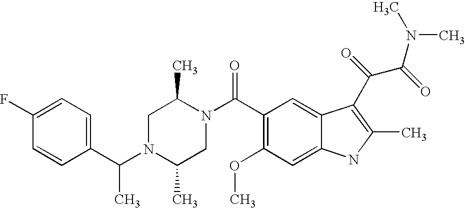 |
| 180 | 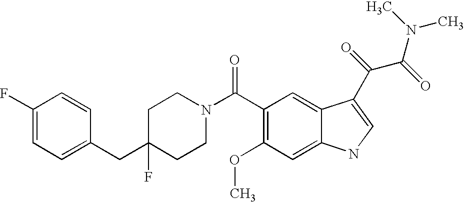 |
| Sigma Compound Product Number S8307 | 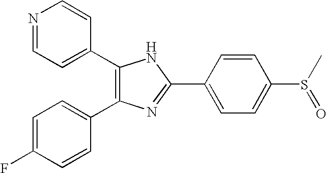 |

Additional compounds are described in published PCT applications WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference in their entirety.

Further additional compounds useful in the practice of the present invention also include, but are not limited to, the compounds shown in Table 3, below.

TABLE 3

| | Citations, each of which is herein |
|---|---|
| Chemical Structure | incorporated by reference. |
| 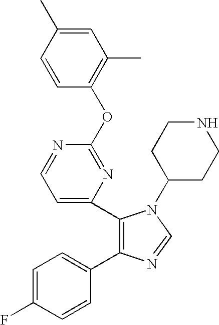 | WO-00166539, WO-00166540, WO-00164679, WO-00138324, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T.F., et. Al., Bioorg. Med. Chem. 5:49 (1997); Adams, J. L., et al., Bioorg. Med. Chem. Lett. 8:3111-3116 (1998) |
| 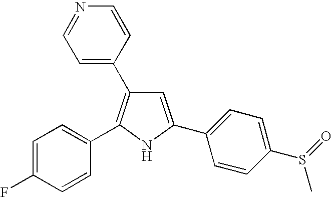 | De Laszlo, S. E., et. Al., Bioorg Med Chem Lett. 8:2698 (1998) |
| 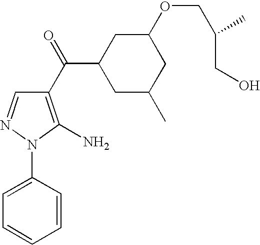 | WO-09957101; Poster presentation at the 5^{th} World Congress on Inflammation, Edinburgh, UK. (2001) |
| 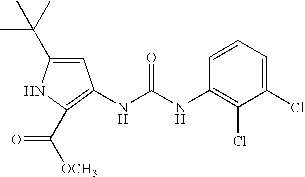 | WO-00041698, WO-09932110, WO-09932463 |
| 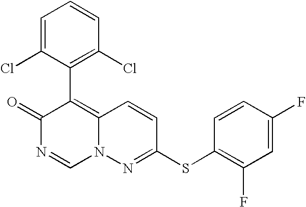 | WO-00017204, WO-09964400 |
| 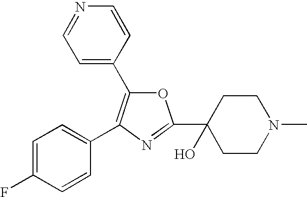 | Revesz. L., et. al., Bioorg Med Chem Lett. 10:1261 (2000) |
| 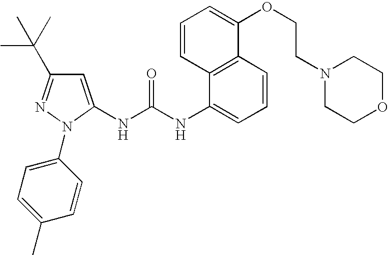 | WO-00207772 |
| 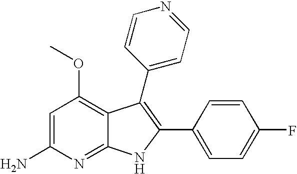 | Fijen, J. W., et al., Clin. Exp. Immunol. 124:16-20 (2001); Wadsworth, S. A., et. al., J. Pharmacol. Expt. Therapeut. 291:680 (1999) |
| 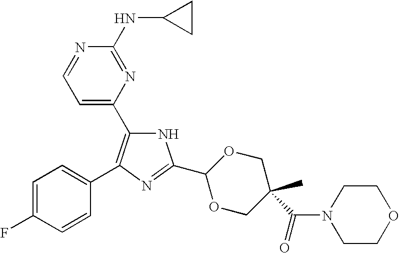 | Collis, A. J., et al., Bioorg. Med. Chem. Lett. 11:693-696 (2001); McLay, L. M., et al., Bioorg Med Chem 9:537-554 (2001) |
| 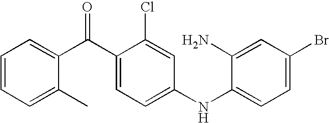 | W000110865, W000105749 |

Additional guidance regarding p38 MAPK inhibitory compounds is found in U.S. patent application Ser. No. 09/575,060 (now U.S. Pat. No. 6,867,209), Ser. No. 10/157,048 (now U.S. Pat. No. 6,864,260), Ser. Nos. 10/146,703, 10/156,997, and 10/156,996, all of which are hereby incorporated by reference in their entirety. The compounds described above are provided for guidance and exemplary purposes only. It should be understood that any modulator of a p38MAP kinase that plays a role in the genesis and maintenance of the MM disease state is useful for the invention provided that it exhibits adequate activity relative to the targeted protein.

Utility and Administration

The methods and compositions of the invention are successful to treat or ameliorate MDS in humans. As used herein, “treat” or “treatment” include effecting postponement of development of undesirable conditions and/or reduction in the severity of such symptoms that will or are expected to develop. Treatment includes ameliorating existing symptoms, preventing additional symptoms, ameliorating or preventing the underlying metabolic causes of symptoms, preventing the severity of the condition or reversing the condition, at least partially. Thus, the terms denote that a beneficial result has been conferred on a subject with MDS.

At the present time, there is no specific treatment for MDS approved by the FDA. Therapy for MDS typically comprises supportive and transfusions for the sickest patients, the administration of growth factors to promote hematopoiesis, and/or the administration of chemotherapeutic agents to control the cellular proliferation observed in MDS bone marrow. As discussed here, the administration of the p38 MAPK inhibitors of the present invention have utility a chemotherapeutic agents for MDS.

The p38 MAPK inhibitors described serve to reduce pathological cytokine levels, increase hematopoiesis, enhance apoptosis of malignant clones, and reduce neoangiogenesis. The p38 MAPK inhibitors described show good safety profiles and present excellent possibilities to combine the use of such inhibitors with other forms of treatment for MDS.

Theoretically, the use of p38 inhibition as a therapy for MDS may be effective because the inhibitors reverse cytokine inhibition of hematopoietic progenitor growth, block marrow cytokines overproduced in MDS, inhibit the negative effects of TNF-alpha, block MMP and VEGF production, potentiate caspase-mediated apoptosis, and perhaps by blocking FasL expression in the MDS clone.

Treatment generally comprises “administering” a subject compound which includes providing the subject compound in a therapeutically effective amount. “Therapeutically effective amount” means the amount of the compound that will treat MDS by eliciting a favorable response in a cell, tissue, organ, system, in a human. The response may be preventive or therapeutic. The administering may be of the compound per se in a pharmaceutically acceptable composition, or this composition may include combinations with other active ingredients that are suitable to the treatment of this condition. The compounds may be administered in a prodrug form.

The manner of administration and formulation of the compounds useful in the invention and their related compounds will depend on the composition of the compound, the nature of the condition, the severity of the condition, the particular subject to be treated, and the judgment of the practitioner; formulation will also depend on mode of administration. For example, if the compounds are “small molecules,” they might be conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like. This method is preferred if the subject can tolerate oral administration.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Intravenous administration is preferred for acute conditions; generally in these circumstances, the subject will be hospitalized. The intravenous route avoids any problems with inability to absorb the orally administered drug.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

Thus, the compounds useful in the method of the invention may be administered systemically or locally. For systemic use, the compounds are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compounds may be administered in a cyclical manner (administration of compound; followed by no administration; followed by administration of compound, and the like). Treatment will continue until the desired outcome is achieved. In general, pharmaceutical formulations will include an active ingredient in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc.

Pharmaceutical compositions can be in the form of sterile, non-pyrogenic liquid solutions or suspensions, coated capsules, suppositories, lyophilized powders, transdermal patches or other forms known in the art.

Biodegradable films or matrices may be used in the invention methods. These include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery may include osmotic minipumps; sustained release matrix materials such as electrically charged dextran beads; collagen-based delivery systems, for example; methylcellulose gel systems; alginate-based systems, and the like.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

Liposomes may also be used as a vehicle, prepared from any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids may also be used.

The dosages of the compounds of the invention will depend on a number of factors which will vary from subject to subject. However, it is believed that generally, the daily oral dosage in humans will utilize 0.1 μg-5 mg/kg body weight, preferably from 1 μg-0.5 mg/kg and more preferably about 1 μg-50 μg/kg. The dose regimen will vary, however, depending on the compound and formulation selected, the condition being treated and the judgment of the practitioner. Optimization of dosage, formulation and regimen is routine for practitioners of the art.

Examples of suitable chemotherapeutic agents for the treatment of MDS include Suitable chemotherapeutic agents include melphalan, vincristine, bischloroethylnitrosourea, melphalan, cyclophosphamide, and prednisone; vincristine, doxorubicin, and dexamethasone, thalidomide, CC-1088 (SelCiD), velcade, Epo, G-CSF, GC-CSF, Bevacizumab (α-VEGF), AG3340 (MMPI), FLT3 antagonists/mixed TKI's, Zarnestra (FTI's), AKT inhibition, Trisenox, Revlimid, (CC-5013, IMiD) and ICL670. Those of ordinary skill in the art are familiar with the dosing regimes used with these chemotherapeutic agents.

Diagnostic Utility

As discussed above, a variety of cytokines were expressed at elevated levels in patients presenting MDS. These cytokines include IL-6, IL-8, IL-Ira, GCSF, GRO, MIP1-beta, MCP1, MDC, eotazin-2, IL-3, MIP1-alpha, BDNF, TIMP-1 and TARC. (See Table 4).

TABLE 4
Cytokine Array analysis showing factors secreted by bone
marrow mononuclear cells which that are induced TNF
and inhibited by p38 MAPK inhibition.

| BMMNC | | | P38 MAPK |
|---|---|---|---|
| Cytokines | General Function | TNF induction | inhibition |
| 1. IL-6 | See above. | strong | strong |
| 2. IL-8 | See above. | weak | strong |
| 3. IL-1ra | IL-1 receptor | strong | weak |
| | antagonist, co- | | |
| | expressed with IL-1 | | |
| | during inflammation. | | |
| 4. GCSF | Stimulates the | strong | weak |
| | differentiation of | | |
| | progenitor cells into | | |
| | granulocytes. | | |
| 5. GRO | See above. | strong | strong |
| 6. MIP1-beta | Macrophage | strong | weak |
| | inflammatory protein, | | |
| | produced by activated | | |
| | macrophages, induces | | |
| | the expression of | | |
| | other pro- | | |
| | inflammatory | | |
| | cytokines. | | |
| 7. MCP1 | See above. | strong | strong |
| 8. MDC | A chemoattractant | strong | strong |
| | causing neutrophilic | | |
| | infiltration at sites of | | |
| | inflammation. | | |
| 9. Eotaxin-2 | Induces chemotaxis of | strong | strong |
| | basophils and | | |
| | eosinophils. | | |
| 10. IL-3 | See above. | strong | strong |
| 11. MIP1-alpha | Similar to MIP1-beta | strong | weak |
| | Known neuron- | | |
| | specific factor. | | |
| 12. BDNF | Serves as a growth | strong | strong |
| | factor for neurons. | | |
| 13. TIMP-1 | Tissue inhibitor of | strong | weak |
| | metalloproteinases, | | |
| | also serves as growth | | |
| | factor for many cell | | |
| | types. | | |
| 14. TARC | T-cell specific | strong | strong |
| | chemokine. | | |

Individuals suspected of suffering from MDS can be more accurately diagnosed and their conditions monitored by following expression levels of these cytokines over time. Standard methods of obtaining diagnostic samples and the test of such samples are used. For example samples of bone marrow can be obtained from long bones and subjected to cytokine array analysis using kits commercially available. Such diagnostic methods can be employed to confirm a diagnosis of MDS and to follow the effectiveness of treatment protocols designed to ameliorate the negative effects of MDS on a patient.

### EXAMPLES

The following examples describe experiments to evaluate the effectiveness of p38 MAPK inhibitors as a treatment for MDS in a patient in need thereof. Table 2 lists a number of compounds that generally exhibit p38 MAPK activity, preferred embodiments exhibit a relative IC₅₀ value of less than 5 nM in an assay similar to the phosphorylation assay disclosed above (see Kumar). The compounds listed in Table 2 exemplify the compounds generically disclosed herein. Moreover, the data discussed below is representative of the genus of p38 MAPK inhibitors disclosed herein. The results discussed below are thought to be obtainable using any of the p38 MAPK inhibitors disclosed herein. As such, the data provided demonstrates that the genus of p38MAPK inhibitor compounds disclosed herein are useful in the disclosed methods of treating MDS. The Sigma-Aldrich® under product number S8307 compound is 4-(4-Fluorophenyl)-2-(4-methylsulfinylphenyl)-5-(4-pyridyl)-1H-imidazole, which is known in the literature as a p38 MAPK modulator and commercial available. This compound is available as a positive control in a p38 MAPK inhibition assay. The following examples are offered to illustrate but not to limit the invention.

### Example 1

Myelosuppresive Effects of Interferon α and Transforming Growth Factor β Can be Reversed by a Novel p38 MAPK Inhibitor Through Inhibition of Cell Cycle Arrest

Cytokines play important roles in the regulation of normal hematopoiesis and a balance between the actions of hematopoietic growth factors and myelosuppressive factors is required for optimal production of cells of different hematopoietic lineages. Even though the effects of Type I Interferons (IFNs α,β) and Transforming Growth Factor βs (TGF βs) as negative regulators of hematopoiesis are well documented, the exact molecular mechanisms by which such effects occur remain unknown. Previous studies have shown that pharmacological inhibition of the p38 MAPK with commercially available inhibitors SB203580 and SB 202190 was able to reverse the myelosuppresion caused by IFN and TGF β. These inhibitors cannot be used in human studies due to toxicity and are also questioned for their selectivity in inhibiting the p38 MAPK. Thus, to confirm the role of p38 MAPK in regulating hematopoiesis, experiments are conducted with a p38 MAPK inhibitor, a potent and selective inhibitor of p38 α. The p38 MAPK inhibitor also performs very similarly in animal and cell models to a p38 inhibitor now in the clinic. The results show that the p38 MAPK inhibitor is able to inhibit p38 MAPK selectively in primary human erythroid progenitors (at CFU-E stage of maturation) and suppress activation of downstream kinase MapKapK-2 after IFN α stimulation. In methycellulose clonogenic assays with mobilized CD34⁺ cells, IFN-α treatment results in marked suppression of both erythroid (BFU-E) and myeloid (CFU-GM) colonies, which can be reversed in the presence of the p38 inhibitor. In a similar manner TGF-β2 is not able to effectively inhibit both erythroid and myeloid colonies in the presence of p38 blockade by a disclosed p38 MAPK inhibitor. In further studies, it is demonstrated that the primary mechanism by which the p38 MAPK pathway mediates IFN mediated hematopoietic suppression is by regulation of cell cycle progression and is unrelated to induction of apoptosis. Treatment with p38 inhibitors leads to significantly lesser numbers of cells in G0/G1 phase of cell cycle arrest induced by exposure to IFN α. Altogether, these findings confirm that the p38 MAPK signaling pathway is a common effector for type I IFN and TGF beta signaling in human hematopoietic progenitors and plays a critical role in the induction of the suppressive effects of these cytokines on normal hematopoiesis. These studies also provide a rationale for the use of a p38 inhibitor in cytokine mediated hematological diseases such as MDS.

### Example 2

### Novel P38 MAP Kinase Inhibitor and Anti-p38 RNA Interference as Potential Therapeutic Approach in Myelodysplastic Syndromes

Cytokines such as TNF-α, IFN-γ and others have been implicated in the pathogenesis of ineffective hematopoiesis in MDS and are thought to lead to the high rate of apoptosis in hematopoietic progenitors. The p38 Mitogen Activated Protein Kinase (MAPK) is an evolutionary conserved enzyme that is involved in many cellular processes including stress signaling. It was previously shown that the p38 MAP kinase is strongly activated by IFNs, TNF-α, TGF-β and other inhibitory cytokines in normal primary hematopoietic progenitors and plays an important role in the negative regulation of normal hematopoiesis. In this study, the role of the p38 MAPK in the pathogenesis of MDS and its inhibition as a potential therapeutic strategy in this disease is studied.

p38 MAPK inhibition is achieved by the use of a novel p38 inhibitor, a specific inhibitor of p38 MAP kinase α, which performs very similarly in animal and cell models to a p38 inhibitor now in the clinic. Primary hematopoietic cells are also transfected with florescent labeled siRNAs against p38 and successfully downregulated the levels of the protein. Using these approaches, it is demonstrated that pharmacological inhibition of the p38 MAPK reversed the growth inhibitory effects of TNF α and IFN γ on erythroid and myeloid colony formation. This reversal of TNF α mediated inhibition correlates with significant reduction of apoptosis seen in human hematopoeitic progenitors pretreated with a p38 inhibitor.

Having established the importance of p38 MAPK in cytokine mediated inhibition of normal hematopoiesis, colony forming assays are performed with bone marrow CD34⁺ cells from 8 patients with MDS in the presence of either pharmacologic or siRNA based inhibitors of p38. All patients have refractory cytopenias with multilineage dysplasia. The data indicated that p38 MAPK inhibitor treatment strongly enhances both erythroid and myeloid colony formation in MDS CD34⁺ bone marrow cells in vitro. This increase is not observed when these progenitors are grown in the presence of negative controls—SB 202474 and the MEK inhibitor PD 98059. Similarly, an increase in hematopoietic colony formation, though of a lesser magnitude is seen when MDS bone marrow progenitors are transfected with siRNAs against p38 MAPK.

To further determine the role of cytokines in the pathogenesis of MDS, bone marrow derived sera from the same MDS patients is used. These studies show that exposure to patient derived sera leads to the phosphorylation/activation of p38 MAPK in normal hematopoietic progenitors when compared to sera from healthy volunteers. These studies also demonstrate that bone marrow derived sera from MDS patients can inhibit erythroid and myeloid colony formation of normal hematopoietic progenitors. This inhibition can be reversed by blocking p38 MAPK using p38 inhibitors, such as small molecule inhibitors and siRNAs directed against p38 MAPK. This finding confirms the role of marrow cytokine/serum factors in the ineffective hematopoiesis seen in MDS and suggests the importance of p38 MAPK activation in this phenomenon.

Thus these studies show the p38 MAPK is a common effector of inhibitory cytokine signaling in normal and MDS hematopoietic cells. These results provide a strong rationale for using p38 inhibitors as a novel treatment strategy for MDS.

### Example 3

### Inhibition of p38 MAPK by A p38 MAPK Inhibitor Suppresses TNF Generation and Promotes CD34+ Cell Survival in an In Vitro MDS Cell Culture Model

Progress in the development of more effective therapeutics for myelodysplastic syndrome (MDS) has been limited by the lack of targets critical to the pathobiology of the disease. Ineffective hematopoiesis in MDS is characterized by accelerated proliferation and premature apoptotic death of progenitors and their progeny that is potentiated by the local generation of inhibitory molecules, including TNFα, TGFβ, FasL, and VEGF. To identify upstream regulatory signals that may coordinate activation of inhibitory molecules, an in vitro cell culture model incorporating a CD34+ MDS cell line isolated from a RAEB-t patient, normal bone marrow stromal cells (BMSC), and/or bone marrow mononuclear cells (BMMNC) is used to determine effects of cell-cell interactions on secretion of inhibitory hematopoietic cytokines. The role of p38 MAP kinase, a regulatory kinase involved in the convergence of inhibitory cytokine activation and signaling, is evaluated in this interaction. It is found that p38 MAPK is induced under basal culture conditions in the MDS cell line and is further activated by TNFα or TGFβ. In all cases, p38 activation is reduced by p38 MAPK inhibition using a potent and specific inhibitor of p38α activity. The p38 MAPK inhibitor does not directly block p38 activation, suggesting a feedback loop is interrupted when p38 kinase activity is inhibited in MDS cells. To determine effects of cell interactions, the MDS cell line is co-cultured with either BMSC, BMMNCs or both from normal donors, and TNFα and FasL secretion are measured after 3 days incubation. TNF-α and FasL are detected in culture supernatants when the MDS cell line is co-cultured with BMMNC but not when co-cultured with BMSC. TNFα secretion by BMMNCs is dependent on MDS cell contact and is significantly inhibited by addition of the p38 MAPK inhibitor. The addition of BMSC to the MDS and BMMNC co-culture prevents TNFα elevation, suggesting BMSCs as a dominant source for anti-inflammatory signal(s). VEGF, FGF-β, TGFβ2, BDNF, TIMP-1, TIMP-2 and IL-6 secretion by BMSC is induced by MDS co-culture, whereas the p38 MAPK inhibitor blocked cytokine induction. To determine the effects of a p38 MAPK inhibitor and MDS clone-induced BM cytokine secretion on normal CD34⁺ proliferation, BMMNCs and BMSC are co-cultured in transwell inserts in the presence or absence of the MDS cell line with or without the we co-cultured. CD34⁺ proliferation is assessed in cells cultured in outer wells. CD34⁺ progenitors proliferate in culture at the same rate as those co-cultured with BMSC, BMMNC and MDS for 6 days. At longer intervals, viability of progenitors cultured with the MDS line declined, whereas treatment with the p38 MAPK inhibitor abrogates the decrease in CD34⁺ viability. These results implicate p38α as a critical target in the induction of pro-apoptotic cytokines in MDS, and that selective inhibition of p38 MAPK by a disclosed p38 MAPK inhibitor provides a novel therapeutic strategy for MDS treatment.

### Example 4

### p38 Inhibitor Therapy and Supportive Care

A patient 75 years of age presenting symptoms of MDS seeks treatment. The symptoms include refractive anemia (RA). The patient also suffers from renal and hepatic disease. The complicating disease states contraindicate chemotherapy for the MDS. Accordingly, supportive care in the form of transfusions of red blood cells and/or platelets with antibiotics and a p38 inhibitor is administered. The patient receives periodic transfusions to alleviate the RA type together with periodic doses of the p38 inhibitor to retard the progression of the MDS. The patient's anemia is thus controlled.

### Example 5

### p38 Inhibitor Therapy and Growth Factors

A 70 year old man is referred with a diagnosis of “chronic anemia”. Liver and thyroid studies are normal. No splenomegaly or hepatomegaly are present. At the time of referral, WBC=4.3×107/L, Hb=7.g g/dl, MCV=105 fl, RDW=12.4, Platelets=1985×107/L. A bone marrow biopsy and aspirate are performed. Cytogenetic studies are performed from the marrow aspirate, and are reported as 46, XY (100%). A diagnosis of MDS, refractory anemia with ringed sideroblasts (RARS) is made.

The patient is administered erythropoietin (EPO) in combination with a p38MAPK inhibitor. The patient's chronic anemia resolves.

### Example 6

### p38 Inhibitor Therapy and Chemotherapy

A patient presenting symptoms of RAEB is provided an effective course of chemotherapy comprising idarubicin and a p38 inhibitor. The symptoms of the RAEB resolve.

### Example 7

### p38 Inhibitor Therapy and Blood Stem Cell Transplant

A patient presenting symptoms of refractory anemia with excess blasts in transformation (RAEB-t). The subject undergoes a non-myeloablative transplant using lower doses of chemotherapy to prepare the patient for transplant. Prior to and in conjunction with the transplant, the patient also receives an effective dose of a p38 MAPK inhibitor. The transplanted stem cells adapt to the marrow of the subject and the symptoms of RAEB-t are resolved.

### Example 8

### p38 MAPK Inhibitors Inhibit Apoptosis and Stimulate Colony Formation by CD34+Progenitors Derived from Low Risk Myelodysplastic Syndrome Bone Marrow

Cells and Reagents

Human bone marrow mononuclear cells (BMMNC) and CD34+ cells were isolated from bone marrows of normal or MDS patients, after approval of informed consent by the institutional review boards of UT Southwestern Medical School, the Dallas VA Medical Center, the University of Arizona College of Medicine and the University of South Florida. Erythroid progenitors at the CFU-E level of differentiation were grown in Iscove's Modified Dulbecco Medium (IMDM, Cambrex; Walkersville, Md.) supplemented with 30% fetal calf serum, 10 ng/ml interleukin-3 (IL-3), 2 IU/ml recombinant human erythropoietin (Epo), 20 ng/ml granulocyte-colony-stimulating factor (GM-CSF), and 50 ng/ml stem cell factor (SCF), all from R&D Systems (R&D Systems; Minneapolis, Minn.). Human recombinant TNFα was also obtained from R&D Systems. Antibodies against MapKapK-2 and the phosphorylated forms of p38 and MapKapK-2 were obtained from Cell Signaling Technology Inc. (Beverly, Mass.). Antibodies against p38α were purchased from Santa Cruz Biotechnology (Santa Cruz, Calif.).

The p38α MAPK inhibitor compound 57 was synthesized by Medicinal Chemistry (Scios, Inc., Fremont, Calif.). compound 57 has an IC₅₀ of 9 nM for inhibition of p38α based on direct enzymatic assays, about 10-fold selectivity for p38α over p38β, and at least 2000-fold selectivity for p38α over a panel of 20 other kinases, including other MAPKs. No significant affinity was detected in a panel of 70 enzymes and receptors. In a cell based assay for inhibition of LPS-induced TNFα secretion in whole human blood, an IC₅₀ of 1.3 μM is observed.

BMMNC Isolation

Primary human bone marrow mononuclear cells (BMMNC) were obtained from healthy volunteers and MDS patients after IRB approved informed consent. BMMNC were isolated by Ficoll-Paque density centrifugation. Whole blood was diluted 1:1 with IMDM+2% FBS and 10 ml of diluted sample was layered on to 15 ml Ficoll-Paque (Stem Cell technologies) in a 50 ml conical tube at room temperature. The tube was centrifuged at 400 g for 30 min. The top plasma layer was discarded while the whitish mononuclear layer was transferred to a 17×100 mm polystyrene tube. Cells were washed with 10 ml of IMDM+2% FBS twice before resuspending the cells in 1 ml IMDM+2% FBS.

CD34+ Progenitor Isolation

CD34+ progenitor cells were obtained by positive immunomagnetic selection from normal or MDS BMMNC (Miltenyi Biotec, Inc; Auburn, Calif.). A total of 2×10 BMMNC cells were resuspended in 600 μL wash buffer [phosphate buffered saline (PBS) supplemented with 0.5% bovine serum albumin (BSA) and 2 mM EDTA, pH 7.2]. FcR Blocking Reagent (200 μL) was added to the cell suspension to inhibit unspecific or Fc-receptor binding of CD34 MultiSort MicroBeads to non-target cells. CD34+ cells were labeled by adding 200 μL CD34 MultiSort MicroBeads, mixed well and incubated for 30 minutes at 4-8° C. The cell suspension was placed in MS columns (combined with the appropriate Column Adapter) in the magnetic field of the MACS Separator. Any unlabeled cells were allowed to pass through by rinsing 3× with 500 μL of wash buffer. To release any bound CD34+ labeled cells, the magnetically labeled cells were incubated with 20 μL MACS® MultiSort Release Reagent per mL of cell suspension for 10 minutes at 4-8° C. CD34+ cells from released fraction were resuspended with 40 μL wash buffer containing 60 μL MACS MultiSort Stop Reagent, washed twice and then finally resuspended in IMDM containing 20% FBS. The purity of the CD34+ isolated cells was verified by flow cytometry.

Western Analysis

CD34+ cells were lysed in phosphorylation lysis buffer as previously described. Cell lysates were resolved on SDS-PAGE and transferred to nitrocellulose membranes (Invitrogen). In the experiments in which the effects of compound 57 were examined, DMSO (diluent)-treated cells were used as control. Western Analysis was performed as previously described.

Apoptosis Assay

Normal CD34+ cells were resuspended in IMDM media in the presence and absence of 20 ng/ml TNFα and 100 nM compound 57 for 24 hours. Apoptotic cells were evaluated by flow cytometry after staining with Annexin V-Alexa Fluor 488 dye (BD Bioscience, San Jose, Calif.). Necrotic cells were visualized in the same assay by co-staining with nucleic acid dye, Sytox green (Vybrant Apoptosis Kit, Molecular Probes Inc., Carlsbad, Calif.). MDS BMMNC were cultured in IMDM media with 20% FBS in the presence and absence of 500 nM compound 57 for 48 hrs. Apoptosis was analyzed by flow cytometry after staining with Annexin V-PE and Propidium iodide, both from BD Bioscience (San Jose, Calif.). Flow samples were analyzed with a FACSCalibur laser flow cytometer and Cell Quest software (BD Bioscience).

Hematopoietic Progenitor Cell Assays

All participants in the study signed informed consent, approved by the institutional review board of UT Southwestern and Dallas VA Medical Center. Hematopoietic progenitor colony formation was determined by clonogenic assays in methylcellulose, as detailed in previous studies. A total of 1×10⁴ isolated CD34+ progenitor cells in 0.4 ml IMDM+2% FBS were resuspended in 4 ml MethoCult® (Stem Cell Technologies) and vortexed vigorously. The CD34+ cell:methylcellulose mixture was dispensed into 35 mm culture dishes (1×10³ cells/35 mm plate) using sterile 3 cc syringe with attached sterile 16-gauge blunt-end needle. Cell cultures were incubated at 37° C., 5% CO₂ in air and >95% humidity for 14-16 days. Granulocyte/macrophage colony-forming (CFU-GM) units and erythroid burst forming units (BFU-E) from different samples were scored on day 14 of culture.

### Results

Compound 57 Inhibits TNFα-Induced p38 MAPK Activation, Apoptosis and Myelosuppression in Normal Hematopoietic Progenitors In Vitro

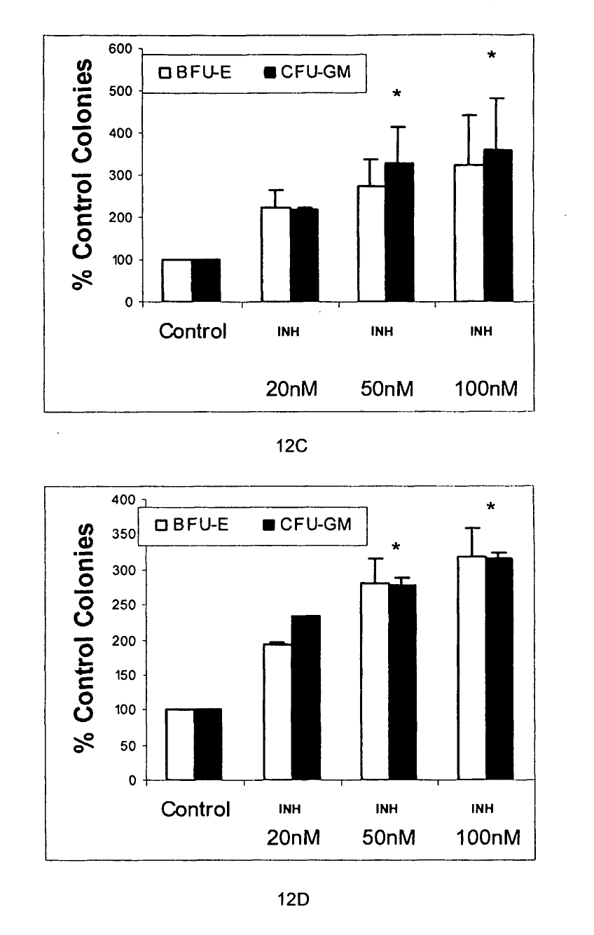

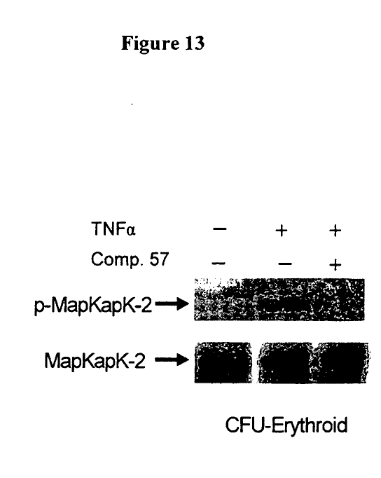

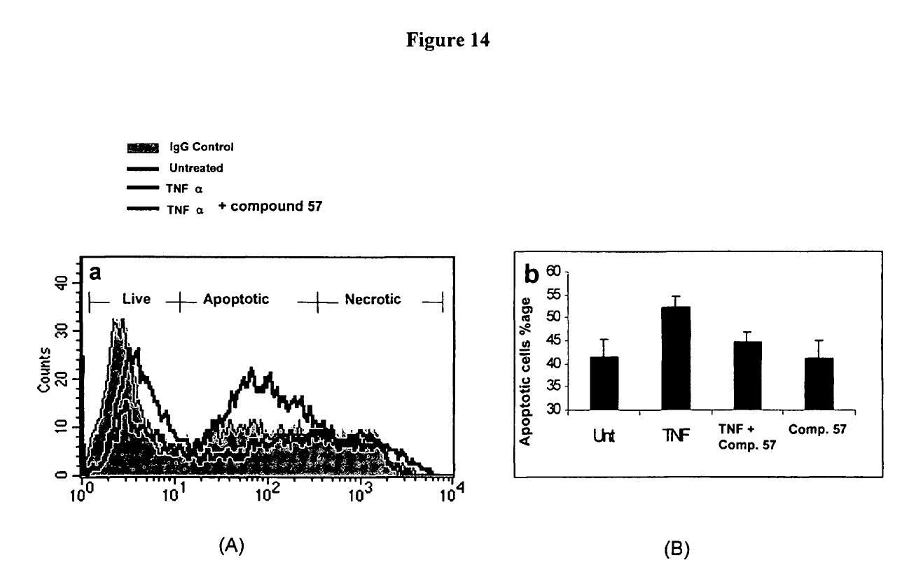

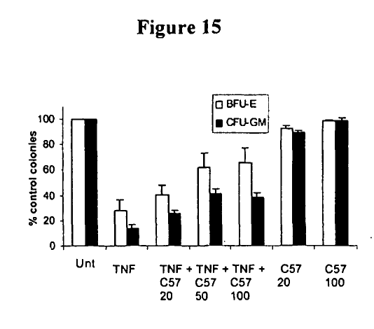

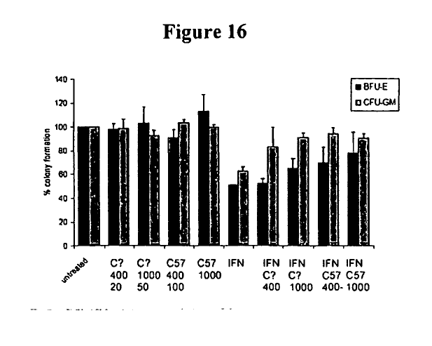

TNFα is a proapoptotic cytokine that has been implicated in the ineffective hematopoiesis seen in MDS. In previous studies, TNFα was found to activate p38 MAPK which leads to the suppression of differentiation in normal hematopoietic progenitor. compound 57 selectively inhibits the activity of p38α, thus blocking the phosphorylation of direct targets including phosphorylation and activation of, MapKapK-2, in human primary hematopoietic precursors (FIG. 13). The effects of compound 57 on TNFα-induced apoptosis and suppression of normal hematopoiesis was therefore examined. Primary human BM derived CD34+ cells were grown in cytokine enriched IMDM media supplemented with 30% fetal calf serum, in the presence or absence of TNFα and with or without compound 57. After 24 hours of exposure, TNFα led to increased apoptosis in the progenitors which was inhibited in the presence of 100 nM compound 57 (FIG. 14). Colony assays with normal CD34+ human hematopoietic cells revealed that TNFα exposure led to a decrease in both erythroid and myeloid colony numbers and this effect was also reversed by compound 57 in a dose dependant manner (FIG. 15). Incubation with as little as 50 nM compound 57 in TNFα-treated CD34+ progenitors achieved about 100% increase in both BFU-E and CFU-GM colony numbers compared to those without compound 57. Similarly, compound 57 was effective in reversing the suppression of both erythroid and myeloid colony formation in normal progenitors after exposure to IFNγ. These results agree with previous published results and suggest that p38 MAPK is commonly activated in hematopoietic progenitors upon stimulation by various proinflammatory cytokines such as TNFα and IFNγ. The cytokine-induced p38 activity in normal progenitors leads to enhanced apoptosis and to the suppression of colony formation, which can all be reversed by treatment with compound 57.

Inhibition of Apoptosis of CD34+ Progenitors in Low Risk MDS Bone Marrow Cells In Vitro

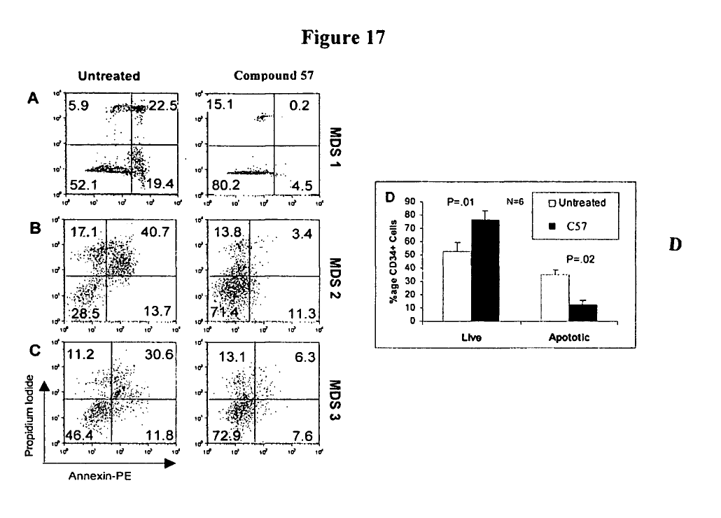

Bone marrow mononuclear cells derived from six patients with MDS (Table 5) were cultured in vitro in the presence and absence of compound 57 (FIG. 17). BM progenitor apoptosis was determined by Annexin V staining on a gated population of CD34+ cells. compound 57 treatment led to a significant decrease in the percentage of apoptotic CD34+ cells after 48 hours of cell culture (P=0.02). Correspondingly, an increase in the number of viable CD34+ cells was also observed in samples treated with compound 57 (P=0.01).

TABLE 5
Clinical characteristics of MDS patients who were sources
of BMMNC used in apoptosis assay with compound 57

| | | WBC | Hgb | Plt | | | IPSS | IPSS |
|---|---|---|---|---|---|---|---|---|
| No. | Age/Sex | (×10⁴/cc) | (gm/dl) | (×10³/cc) | Cytogenetics | Subtype | Score | Grade |
| 1 | 57M | 4.95 | 10 | 337 | Y | RAEB | BMA blasts 0.5 | Int-2 |
| 2 | 56M | 3.03 | 8 | 102 | N | RARS | 0 | Low |
| 3 | 75M | 4.75 | 9.6 | 319 | N | RA | Cytopenia 0.5 | Int-1 |
| 4 | 61M | 4.17 | 8.7 | 442 | N | RAEB | BMA blasts 0.5 | Int-1 |
| 5 | 72M | 7.28 | 7.4 | 399 | N | RARS | 0 | Low |
| 6 | 73F | 4.82 | 8.6 | 311 | N | RCMD-RS | 0 | Low |

Source: University of South Florida

Compound 57 Enhances Hematopoiesis in Purified CD34+ Progenitors Derived From Low Risk MDS Bone Marrow Cells

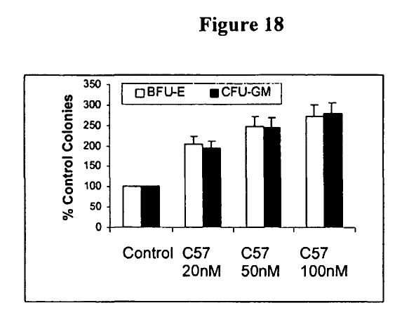

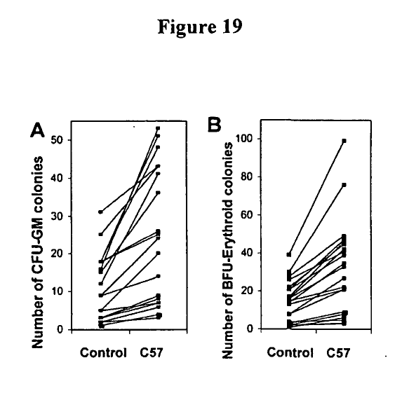

On exposure to compound 57, purified CD34+ progenitors from predominantly low risk MDS patients (Table 6) demonstrated an increase in both erythroid and myeloid colony numbers in vitro (FIG. 18). Consistent with previous studies, untreated MDS CD34+ cells exhibited poor colony forming ability, demonstrating low hematopoietic potential of these cells. Treatment with very low doses of compound 57 (20 nM-100 nM) led to a 2-3 fold increase in myeloid and erythroid colony numbers in MDS progenitors (FIG. 19).

TABLE 6
Clinical characteristics of MDS patients who were sources of CD34+
progenitors used in clonogenic assays with compound 57

| | | WBC | Hgb | Plt | | | IPSS | IPSS |
|---|---|---|---|---|---|---|---|---|
| No. | Age/Sex | (×10⁴/cc) | (gm/dl) | (×10³/cc) | Cytogenetics | Subtype | Score | Grade |
| 1 | 55M | 3.4 | 10 | 146 | N | RA | 0 | Low |
| 2 | 81M | 8.2 | 10 | 239 | N | RA | 0 | Low |
| 3 | 81M | 3.9 | 7.8 | 134 | N | RARS | 0 | Low |
| 4 | 86M | 3.9 | 7.8 | 134 | −Y | RCMD | 0 | Low |
| 5 | 81M | 12 | 9 | 131 | N | RCMD | 0 | Low |
| 6 | 76M | 6 | 7 | 137 | N | RCMD | 0 | Low |
| 7 | 79M | 6 | 11 | 140 | N | RCMD-RS | 0 | Low |
| 8 | 58M | 3 | 12 | 106 | N | RA | 0.5 | Int-1 |
| 9 | 71M | 4.2 | 6 | 130 | N | RCMD | 0.5 | Int-1 |
| 10 | 88M | 2.4 | 8.3 | 155 | N | RCMD | 0.5 | Int-1 |
| 11 | 66M | 5.1 | 11 | 88 | N | RCMD | 0.5 | Int-1 |
| 12 | 56M | 2 | 9 | 12 | N | RCMD | 0.5 | Int-1 |
| 13 | 77M | 2 | 12 | 174 | N | RCMD | 0.5 | Int-1 |
| 14 | 81F | 6.7 | 10 | 155 | −11q | RCMD-RS | 0.5 | Int-1 |
| 15 | 69F | 4 | 8.4 | 145 | −7 | RCMD | 1 | Int-1 |
| 16 | 48F | 5.2 | 8.4 | 95 | −1q, −11q | RAEB | 1.5 | Int-2 |
| 17 | 61M | 1.3 | 8 | 19 | N | RAEB | 1.5 | Int-2 |
| 18 | 78M | 0.6 | 6 | 30 | del 16 (q22) | RCMD | 1.5 | Int-2 |
| 19 | 55M | 0.3 | 8 | 4 | −20 | RCMD | 2 | Int-2 |

Source: Univeristy of Texas Southwestern Medical School

### Discussion

This report shows that the activation of p38 MAPK in normal hematopoeitic progenitors by proinflammatory cytokines such as TNFα leads to increased progenitor apoptosis and to the suppression of both myeloid and erythroid colony formation in clonogenic assays. Isolated CD34+ progenitors from low risk MDS patients have higher apoptotic index and lower hematopoietic potential in vitro compared to normal progenitors. The activation of the apoptotic pathway in isolated MDS cells is possibly due to the stimulation by the different proinflammatory cytokines which are present in the MDS bone marrow. Treatment of MDS progenitors with the p38α inhibitor, compound 57 leads to a significantly enhanced cell viability in vitro and to increased myeloid and erythroid colony formation. The increased colony formation in clonogenic assays of MDS progenitors after compound 57 treatment could be indirect, resulting from the inhibition of CD34+ apoptosis. It could also be due to the direct inhibition of the TNFα-induced myelosuppression of hematopoietic differentiation of CD34+ progenitors. These results suggest that activation of p38 MAPK by proinflammatory cytokines in CD34+ progenitors leads to the suppression of hematopoiesis in low risk MDS.

In low risk MDS, both normal and cytogenetically abnormal hematopoietic clones are found to exist in the marrow. It has been shown that abnormal MDS progenitor clones are resistant to apoptosis and have higher levels of anti apoptotic proteins bcl-2 and bax. It is possible that p38 inhibition may prevent cell death in the susceptible normal progenitors thereby rescuing normal hematopoiesis in the early/low grade stage of this disease. Since most MDS cases are low risk and the morbidity experienced is due to low blood counts, hematopoietic recovery is a major therapeutic goal in treating low risk MDS patients. With disease progression towards high risk stages, normal progenitors gradually undergo apoptosis resulting in a bone marrow comprised mainly of the resistant abnormal clones. Thus examination of the marrow at late stages reveals a low apoptotic index with higher percentages of myeloblasts.

MDS is highlighted by a stromal pathology of still unknown causes, which contributes to the pervasive presence of pro-inflammatory cytokines in the bone marrow. Dysregulation of various cytokines has been implicated in the pathogenesis of MDS. TNFα and IFNs γ, α are myelosuppressive cytokines that have been found to be elevated in serum as well as in the bone marrow of MDS patients. Earlier work has shown that p38 MAPK is activated by all of these cytokines in primary hematopoietic progenitors. Furthermore, p38 MAPK has been shown to function at a critical signaling juncture that links upstream signaling pathways induced by different immunosuppressive cytokines to a common effector pathway that leads to the inhibition of normal hematopoietic progenitor growth. Thus, inhibiting the p38 MAPK pathway with compound 57 may improve progenitor growth and alleviate the myelosuppressive effects of inflammatory cytokines on hematopoiesis.

### Example 9

### Compound 57 Inhibits the Pathological Loop Generation of Proinflammatory Factors in the MDS Bone Marrow Microenvironment

Reagents

Human IL-1β, TNFα, IL-12, IL-18 and TGF-β were from R&D Systems (Minneapolis, Minn.). The human hematopoietic stem cell cytokine panel consisting of stem cell factor (SCF), thrombopoietin (Tpo) and Flt3-ligand (FL) were also from R&D Systems. Fluorochrome conjugated antibodies CD45-FITC, CD34-PerCP, CD3-Pacific Blue, CD19-APCCy7, CD56-PECy7, CD14-APC, IL-1β-PE, TNFα-PE, caspase 3-FITC, phospho-p38-PE and their corresponding fluorochrome-conjugated isotype IgG control antibodies were from BD Bioscience (San Jose, Calif.). Lipopolysaccharide (LPS) was obtained from Sigma (St. Louis, Mo.). Brefeldin A (Golgi Plug) was obtained from BD Bioscience.

The p38α MAPK inhibitor compound 57 was synthesized by Medicinal Chemistry (Scios, Inc., Fremont, Calif.). compound 57 has an IC₅₀ of 9 nM for inhibition of p38α based on direct enzymatic assays, about 10-fold selectivity for p38α over p38β, and at least 2000-fold selectivity for p38α over a panel of 20 other kinases, including other MAPKs. No significant affinity was detected in a panel of 70 enzymes and receptors. In a cell based assay for inhibition of LPS-induced TNFα secretion in whole human blood, an IC₅₀ of 1.3 μM is observed.

BMMNC and BMSC Cell Culture

Primary human bone marrow mononuclear cells (BMMNC) were obtained from MDS patients after IRB approved informed consent from the institutional review boards of Albert Eistein Medical School and the Univerity of South Florida. BMMNC were isolated by Ficoll-Paque density centrifugation. Whole blood was diluted 1:1 with Iscove's Modified Dulbecco Medium (IMDM, Cambrex; Walkersville, Md.) containing 2% FBS and 10 ml of diluted sample was layered on to 15 ml Ficoll-Paque (Stem Cell technologies) in a 50 ml conical tube at room temperature. The tube was centrifuged at 400 g for 30 min. The top plasma layer was discarded while the whitish mononuclear layer was transferred to a 17×100 mm polystyrene tube. Cells were washed with 10 ml of IMDM+2% FBS twice-before resuspending the cells in 1 ml IMDM+2% FBS. Normal BMMNC were obtained cryopreserved from Cambrex (Atlanta, Ga.) and maintained in IMDM+15% FBS containing 50 ng/ml each of SCF, Tpo and FL.

Non-irradiated bone marrow stromal cells (BMSC) from normal donors were obtained from Cambrex and maintained in Myelocult H5100 medium supplemented with 10⁻⁶ M hydrocortisone (Stem Cell Technologies; Vancouver, BC, Canada). BMSC from MDS patients were derived from adherent layers that grew after two weeks in cell cultures of MDS BMMNC in IMDM+10% FBS containing the hematopoietic stem cell cytokine panel. These cells were subsequently maintained and passaged in Myelocult H5100 media.

ELISA

TNFα, IL-6, VEGF, and IFNγ concentrations of the cell culture supernatants were assayed using ELISA kits from BioSource International (Camarillo, Calif.). MCP-1, MMP-2 and MMP-9 concentrations were assayed using ELISA kits from R&D Systems.

Multicolor Flow Cytometry

BMMNC were washed in FBS buffer (PBS containing 1% FBS and 0.09% sodium azide, BD Bioscience) and then stained with fluorochrome-conjugated receptor antibodies for 30 min at RT. Cells were washed twice in FBS buffer and then simultaneously fixed and permeablized in Cytofix/Cytoperm solutionn for 20 min at 4° C. (BD Bioscience). Cells were then washed twice in 1X Cytoperm/Cytowash solution (BD Bioscience) before intracellular staining with either TNFα-PE or IL-1β-PE in Cytoperm/Cytowash solution for 30 min at RT. Cells were washed twice in Cytoperm/Cytowash before resuspending in 1% paraformaldehyde solution in PBS. Cells were analyzed by multicolor flow analysis using the BD LSR II flow cytometer and the FACSDiva software program (BD Bioscience).

For phospho-p38 and caspase 3 staining, normal and MDS BMMNC were first fixed in 1.5% paraformaldehyde solution in PBS for 10 min at RT. Cells were then washed in BSA buffer (PBS+0.1% BSA, BD Bioscience) before resuspending and vortexing in 95% methanol in PBS for 5 min at 4° C. Cells were washed twice with BSA buffer and then simultaneously stained with phospho-p38-PE and activated caspase 3-FITC for 30 min at RT. After washing twice in BSA buffer, cells were resuspended in 1% paraformaldehyde solution in PBS before analyzing by flow cytometry analysis using the BD FACScan and the BD CellQuest software program (BD Bioscience).

Apoptosis Assay

Detection of apoptotic cells was performed by staining with Annexin V-FITC and 7-Amino Actinomycin D (7-AAD) (BD Pharmigen; San Diego, Calif.) BMMNC samples were co-stained with anti-CD34-PECy7 and CD45-APCCy7 to detect apoptosis of CD34+ progenitors (CD34+CD45-cell population). Samples were analyzed by multicolor flow cytometry using a using the BD LSR II flow cytometer and the FACSDiva software program (BD Bioscience) 7-AAD is a nucleic acid dye that is used to exclude nonviable cells in flow cytometric assays. Cells that were Annexin V-PE positive and 7-AAD negative were considered early apoptotic.

cDNA Microarray Analysis

Details of microarray and data analysis have been described previously. The data was normalized using the maNorm function in array package of Bioconductor version 1.5.8. Differential expression values were expressed as the ratio of the median of background-subtracted fluorescence intensity of the experimental RNA to the median of background-subtracted fluorescence intensity of the control RNA. The total BMSC RNA was extracted from cells using Qiagen's RNeasy kit (Valencia, Calif.). Arrays were probed in quadruplicate for a total of 16 hybridizations: control versus TNFα (24 hours), TNFα versus compound 57+TNFα (24 hours), control versus IL-1β (24 hours), IL-1β versus compound 57+IL-1β (24 hours).

Protein Array Analysis

BMMNC (1×10⁶) were stimulated with 2 ng/ml TNFα for 3 days in the presence or absence of 1 uM compound 57. Protein supernatants were subjected to protein array analysis using the 120-cytokine panel RayBio® Human Cytokine Antibody Array C Series 1000 (Raybiotech, Inc). Each array contained duplicate protein samples and the experiment itself was performed independently by two different researchers.

Results

Compound 57 Inhibits LPS-Induced IL-1β Expression in Normal BMMNC

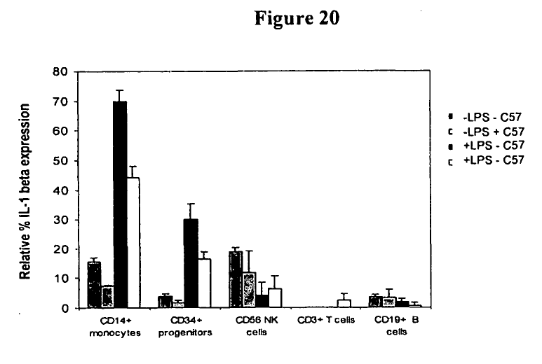

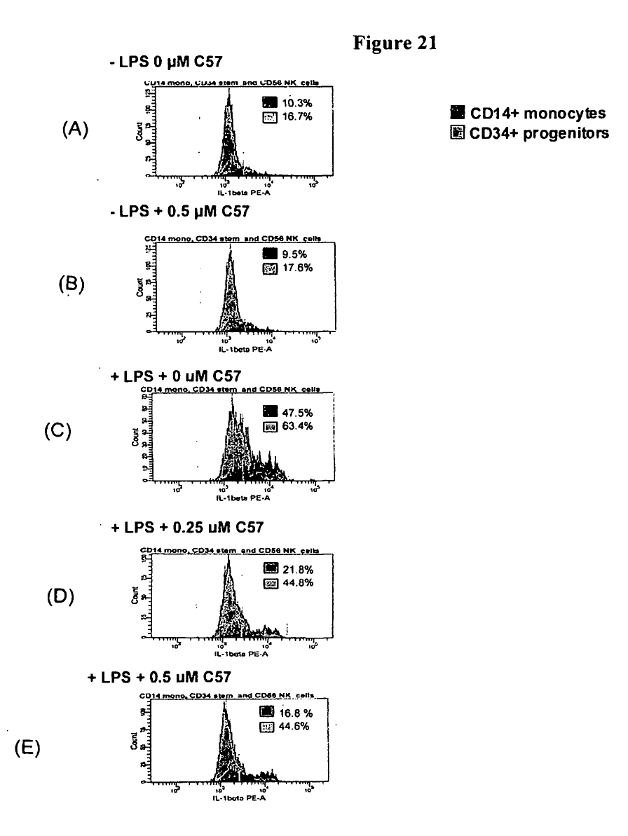

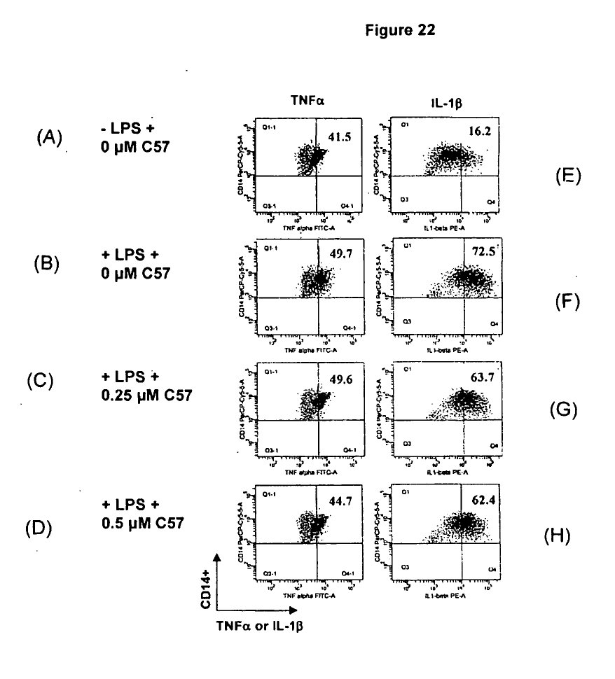

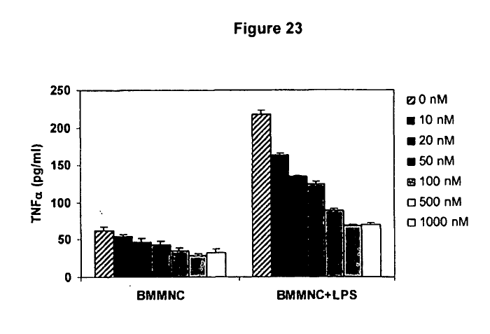

IL-1β is a proinflammatory cytokine that is highly expressed in the bone marrow mononuclear cells of low risk MDS patients compared to normal healthy controls (Appendix 1). The increased expression of IL-1β correlates with the increased p38 activation as well as the increased apoptosis in these cells, as measured by phospho-p38 and caspase 3 staining respectively, through flow cytometry. The specific inducer of proinflammatory cytokine expression in MDS, including that of IL-1β, is still largely unknown. We therefore used LPS as a primary inducer of inflammation to determine whether the p38α inhibitor, compound 57, could inhibit LPS-induced IL-1β expression in normal bone marrow. FIG. 20 shows that IL-1β was induced mainly in CD14+ monocytes and in CD34+ progenitor cells after 4 hours of LPS stimulation. IL-1β expression was not induced after LPS treatment in CD56+ NK cells, CD3+ T cells or in CD19+ B cells. compound 57 effectively reduced the intracellular IL-1β expression in both CD14+ and CD34+ populations as demonstrated by flow cytometry (FIG. 21). Surprisingly, LPS-induced TNFα expression was not detected in any cell population of these same BM samples (data not shown). In peripheral blood, LPS is known to highly induce both TNFα and IL-1β expression in CD14+ monocytes even after only 3 hours of in vivo or in vitro stimulation. Thus to confirm our initial observation, we compared the LPS-induced expression of both TNFα and IL-1β in adherent CD14+ monocyte/macrophage cells isolated from the BM of a different normal donor (FIG. 22). TNFα expression was induced only 1.2-fold after 4 hours of LPS stimulation while IL-1β was induced 4.5-fold in BM CD14+ cells and was dose dependently inhibited by compound 57. Nevertheless, as shown in FIG. 23, LPS-induced TNFα expression was later detected in BMMNC after 24 h.

APPENDIX 1
Relative expression of IL-1beta, phospho-p38, and activated
caspase 3 in BMMNC from low risk MDS patients and normal
controls as determined by flow cytometry
Study #1

| | MDS | % p-p38- | % caspase | IL-1 beta | Patient |
|---|---|---|---|---|---|
| Sample | subtype | positive | 3-positive | expression | Initials |
| Normal 1 | normal | 24 | 55 | 53 | N4 |
| Normal 2 | normal | 31 | 53 | 54 | N5 |
| Normal 3 | normal | 38 | 49 | 50 | N6 |
| MDS 1 | RA | 67 | 76 | 76 | DC1 |
| MDS 2 | RARS | 65 | 77 | 77 | AH |
| MDS 3 | RARS | 57 | 85 | 89 | BG |
| | MDS | p-p38+ | Apoptotic | IL-1 beta | Patient |
| Sample | subtype | cells (%) | cells (%) | expression | Initials |

Study #2

| | | | | | |
|---|---|---|---|---|---|
| Normal 4 | normal | 26 | 67 | 18 | N4 |
| MDS 4 | RA | 50 | 85 | 53 | NB |
| MDS 5 | RA | 39 | 82 | 32 | KB |
| MDS 6 | RA | 40 | 92 | 64 | DC2 |

Study #3

| | | | | | |
|---|---|---|---|---|---|
| Normal 5 | normal | 27 | 64 | 19 | N5 |
| MDS 7 | RCMD | 45 | 70 | 76 | VW |
| MDS 8 | RCMD | 71 | 78 | 67 | JG |

### Compound 57 Inhibits LPS-, IL-1β- and BMSC-Induced TNFα Expression in BMMNC

TNFα is another proinflammatory cytokine which is found to be overexpressed in MDS patients. TNFα expression levels in MDS bone marrow significantly correlates with increased apoptosis of CD34+ progenitor cells. TNFα levels were also found to be inversely correlated with hemoglobin levels, suggesting its relation to the cause of anemia. We therefore next examined whether TNFα expression can be modulated by p38 inhibition with compound 57 in inflammation-stimulated normal or MDS bone marrow cells.

Both basal and LPS-induced TNFα production was detected after 24 h by ELISA from supernatants of normal BMMNC cultures (FIG. 23). compound 57, in a dose dependent manner, potently inhibited the secretion of TNFα from both basal or LPS-induced cell cultures with an IC₅₀ of 50 nM. While TNFα may not have been highly induced early in BM CD14+ cells after 4 h LPS stimulation (FIG. 22), it is possible that IL-1β, which is induced and secreted early under these conditions, could have re-stimulated the same BM cells to secrete TNFα at the later time points as detected by ELISA (FIG. 23).

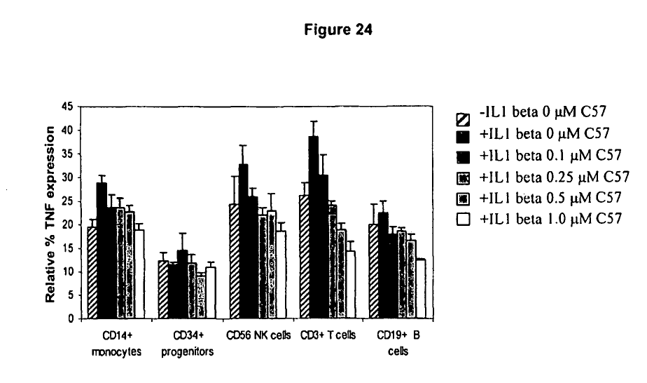

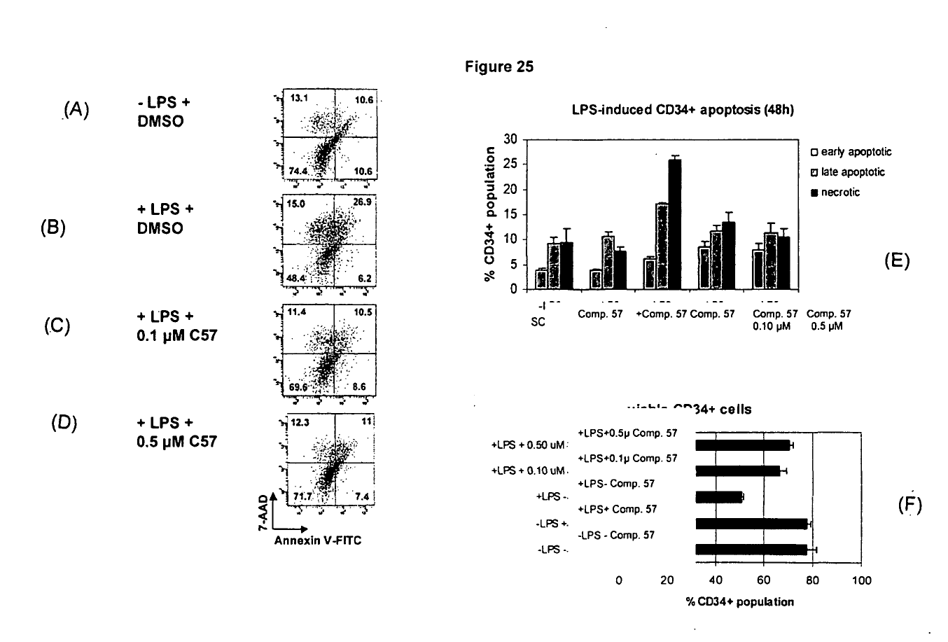

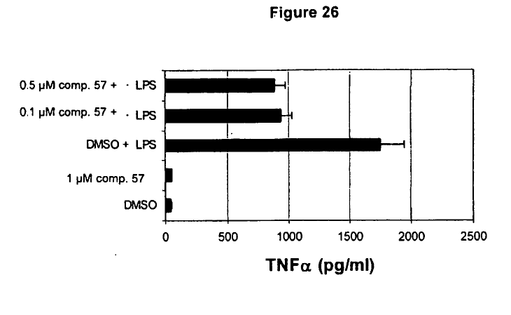

FIG. 24 shows that IL1-β stimulated TNFα expression after 24 hours in BM CD14+ monocytes and CD3+ T cells. TNFα expression in these cells as well as in CD56+ NK and CD19+ B cells was also inhibited by compound 57 in a dose dependent manner, and with TNFα inhibition reaching below basal levels in many cells. Since TNFα expression has also been shown to correlate with increased apoptosis of CD34+ cells in MDS bone marrow, we then examined apoptosis of CD34+ cells in LPS-induced BMMNC after 48 h by co-staining with Annexin V and CD34+. FIG. 25 shows that the total percentage of apoptotic/necrotic cells correlated with the levels of TNFα secreted in the cell cultures (FIG. 26) and that treatment with compound 57 proportionately reduced the levels of TNFα and increased the proportion of viable CD34+ progenitors.

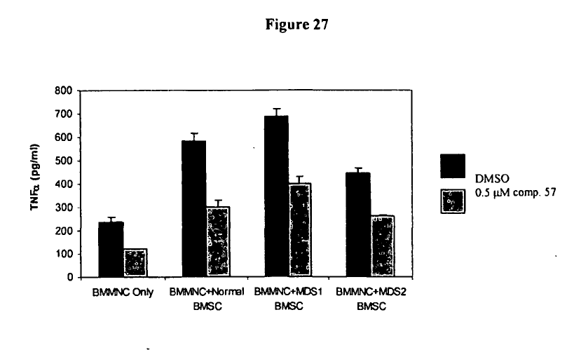

Similarly, BMSC, isolated from normal healthy donors, strongly induced TNFα secretion from BMMNC cocultures, which was similarly inhibited by compound 57 (FIG. 27). BMSC itself does not secrete appreciable amounts of TNFα. To investigate the role of MDS BMSC on the induction of TNFα secretion, we isolated BMSC from two different low risk MDS patients whose BM cells have been found to have increased levels of activated p38 (data not shown). We then cocultured these with BMMNC isolated from normal donors. FIG. 27 shows that MDS stroma was capable of inducing normal BMMNC to secrete TNFα at levels similar to those induced by normal BMSC. This result suggests that MDS stroma is not inherently transformed and may not directly trigger the increased production of proinflammatory cytokines such as TNFα seen in MDS marrows.

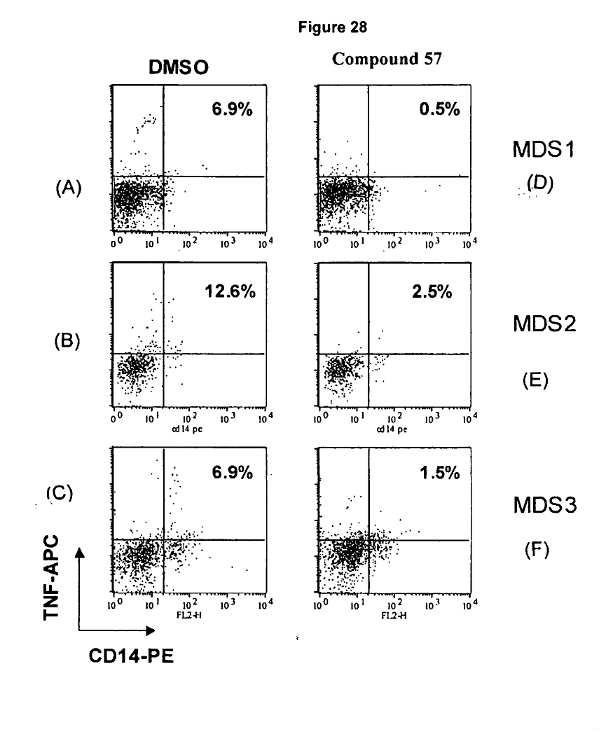

Compared to normal healthy controls, there was an increased proportion of CD14+ monocytes expressing TNFα in BMMNC isolated from low risk MDS patients (data not shown). compound 57 was found to effectively inhibit TNFα expression in CD14+ monocytes in BMMNC isolated from MDS patients (FIG. 28). These results suggest that selective inhibition of p38α by compound 57 either in normal BMMNC which were stimulated by different proinflammatory stimuli or in MDS BMMNC, effectively reduced the secretion and production of the proinflammatory cytokine, TNFα.

### Compound 57 Inhibits the Secretion of Pro-Inflammatory Factors Induced by TNFα or IL-1β from BMMNC or BMSC

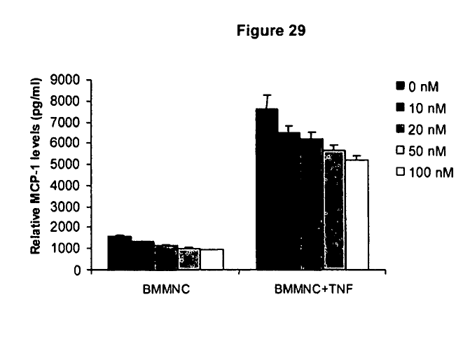

The overproduction of proinflammatory cytokines such as TNFα and IL-1β could lead to the amplification of the initial MDS inflammatory stimuli and thus, to a chronic inflammatory microenvironment in the MDS bone marrow. For instance, TNFα and IL1-β could stimulate the migration and activation of inflammatory leukocytes that secrete these cytokines to the local sites of inflammation. To examine whether such mechanism could be regulated by p38 MAPK, we stimulated BMSC with TNFα or IL-1β for 24 hours in the presence or absence of compound 57 and analyzed the gene expression profile in these cells by Microarray Analysis to look for any p38-regulated genes that might promote inflammation. Surprisingly, we found a number of chemokines that were strongly induced by both IL-1β and TNFα that were also strongly inhibited by compound 57 (Table 1). Among these they include several known chemokines such as CCL2 (monocyte chemoattractant protein-1, MCP-1), CCL7 (MCP-3), CXCL10 (IP-10), CXCL6 (granulocyte chemotactic protein 2) CXCL3 (Gro-gamma), and CXCL1 (Gro-alpha). Most of these chemokines have been recently implicated in promoting adhesion of leukocytes to BM stromal cells. In addition to being induced in BMSC, we also found that MCP-1 protein was highly induced in BMMNC after TNFα stimulation and this was also partly inhibited by compound 57 (FIG. 29). In addition to MCP-1, other proteins that we found through a 120-cytokine panel protein array that were induced in BMMNC and were also inhibited by compound 57 include Eotaxin-2, MDC, IL-3, BDNF, TARC, and TIMP-1 (Table 7).

TABLE 7
Gene microarray analysis of chemokines induced byTNFα and
IL-1β and inhibited by compound 57 in BMSC

| | | | TNFα | C57 + TNFα | | C57 + |
|---|---|---|---|---|---|---|
| Symbol | Other name | Name | (24 h) | (24 h) | IL-1β(24 h) | IL-1β |
| CXCL1 | GROα | Chemokine (CXC) ligand 1 | 40.7 | −2.9 | 125.9 | −1.6 |
| CCL2 | MCP-1 | Chemokine (CC) ligand 2 | 14.2 | −1.7 | 9.9 | −1.4 |
| CXCL6 | GCP2 | Chemokine (CXC) ligand 6 | 12.6 | −5.3 | 138.8 | −1.6 |
| CXCL3 | GROγ | Chemokine (CXC) ligand 3 | 7.2 | −1.6 | 40.6 | 1.3 |
| CCL7 | MCP3 | Chemokine (CC) ligand 7 | 4.0 | −2.2 | 6.2 | −1.5 |
| CXCL10 | IP10 | Chemokine (CXC) ligand 10 | 3.0 | −2.9 | 1.0 | 1.0 |
| CXCL11 | ITAC | Chemokine (CXC) ligand 11 | 2.3 | −1.4 | 1.0 | 0.0 |
| CXCL16 | SR-PSOX | Chemokine (CXC) ligand 16 | −1 | 1.4 | 9.3 | −4.2 |

### Compound 57 Inhibits VEGF and IL-6 Secretion from Normal or MDS BMSC

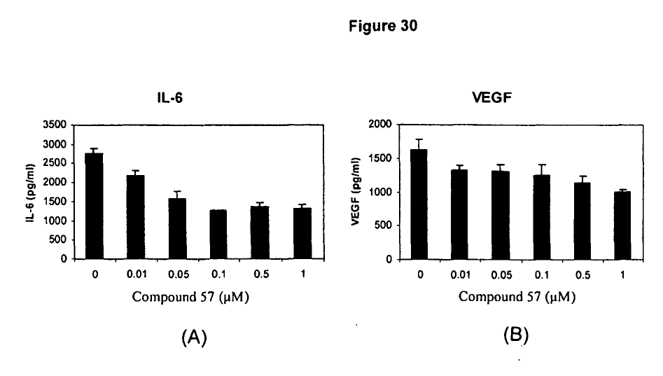

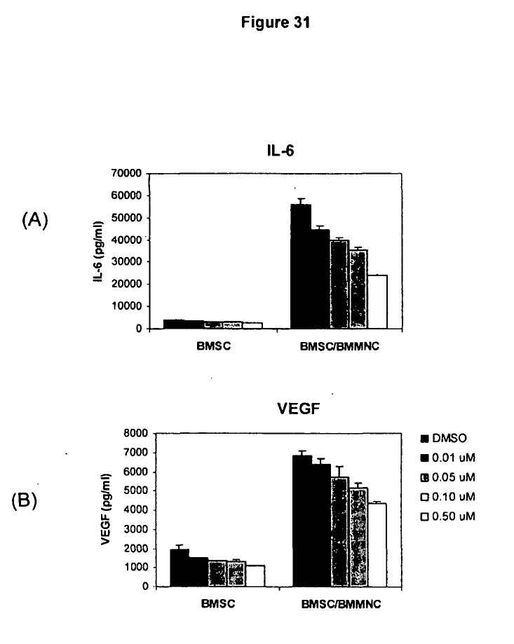

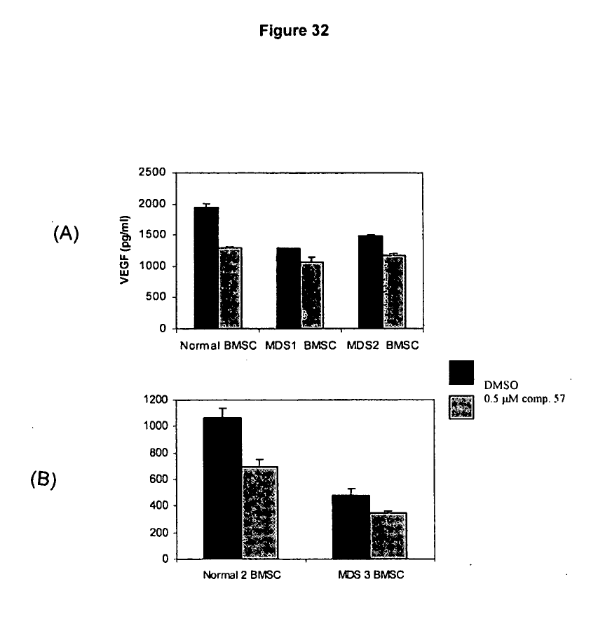

Other proinflammatory factors whose levels have been observed to be significantly higher in MDS marrow include VEGF and IL-6. In normal BM, these cytokines were found to be secreted mainly by BMSC. The production and secretion of basal levels of IL-6 and VEGF were inhibited by compound 57 in a dose dependent manner (FIG. 30). The levels of these cytokines are significantly induced by coculture with normal BMMNC and the stimulated levels were also found to be effectively reduced by treatment with compound 57 (FIG. 31). In support of our earlier assumption that cytokine secretion from MDS stroma is inherently normal, VEGF levels secreted from two different MDS stroma were found to be comparable to, and in fact, may even be lower than those detected from BMSC isolated from different normal controls (FIG. 32). In MDS, VEGF expression has been shown to be proportional to the percentage of MDS blasts and has also been detected by IHC to be specifically expressed by the rapidly proliferating abnormal clones. The concurrent increase of VEGF receptors in these cells also suggest that VEGF production by the undifferentiated blasts could potentially feed into their own proliferation. Inhibition of VEGF production by compound 57 can potentially diminish the proliferation of the undifferentiated MDS blast cells and thus reduce their potential to transformed into leukemic cells.

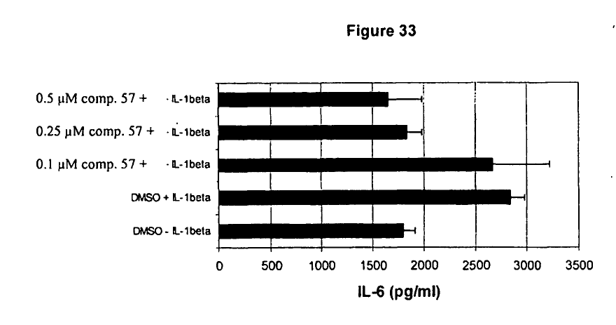

Similarly, IL-6 is known to promote inflammation in other bone marrow diseases such as Multiple Myeloma. IL-6 levels were also found to be significantly higher in RAEB-t patients compared to RA, RAEB and CMML (42). Correspondingly, high levels of IL-100 ng/ml was found to induce IL-6 secretion in BMMNC, and this was effectively decreased by compound 57 treatment (FIG. 33). TNF was also found to induce IL-6 and IL-8 in BMMNC which were both strongly inhibited by compound 57 (Table 8).

TABLE 8
Protein array analysis of TNFα-induced cytokines and
chemokines inhibited by compound 57 in BMMNC

| BMMNC | | TNF | SCIO-469 | # of times |
|---|---|---|---|---|
| cytokines/chemokines | General Function | induction | inhibition | seen |
| 1. IL-6 | One of the major growth- | strong | strong | 2 |
| | promoting factors for | | | |
| | myelomas. | | | |
| 2. IL-8 | Inflammatory cytokine, | weak | strong | 2 |
| | activates neutrophils. | | | |
| 3. IL-1ra | IL-1 receptor antagonist, co- | strong | weak | 2 |
| | expressed with IL-1 during | | | |
| | inflammation. | | | |
| 4. GCSF | Stimulates the | strong | weak | 2 |
| | differentiation of progenitor | | | |
| | cells into granulocytes. | | | |
| 5. GRO | Growth-regulated | strong | strong | 2 |
| | oncogene, inflammatory | | | |
| | cytokine causing the | | | |
| | infiltration of neutrophils and | | | |
| | the subsequent | | | |
| | degranulation and release | | | |
| | of lysosomal enzymes. | | | |
| 6. MIP1-beta | Macrophage inflammatory | strong | weak | 2 |
| | protein, produced by | | | |
| | activated macrophages, | | | |
| | induces the expression of | | | |
| | other pro-inflammatory | | | |
| | cytokines. | | | |
| 7. MCP1 | A monocyte-specific | strong | strong | 2 |
| | chemotactic cytokine, also | | | |
| | activates basophils to | | | |
| | degranulate and release | | | |
| | histamine. | | | |
| 8. MDC | A chemoattractant causing | strong | strong | 2 |
| | neutrophilic infiltration at | | | |
| | sites of inflammation. | | | |
| 9. Eotaxin-2 | Induces chemotaxis of | strong | strong | 2 |
| | basophils and eosinophils. | | | |
| 10. IL-3 | Stimulates the proliferation | strong | strong | 1 (EH) |
| | of immature hematopoietic | | | |
| | cells. | | | |
| 11. MIP1-alpha | Similar to MIP1-beta. | strong | weak | 1 (AN) |
| 12. BDNF | This is a neuron-specific | strong | strong | 1 (EH) |
| | factor. Serves as a growth | | | |
| | factor for neurons. | | | |
| 13. TIMP-1 | Tissue inhibitor of | strong | weak | 1 (AN) |
| | metalloproteinases, also | | | |
| | serves as growth factor for | | | |
| | many cell types. | | | |
| 14. TARC | T-cell specific chemokine. | strong | strong | 1 (AN) |

### Compound 57 Blocks IFNγ Production in IL-12 and IL-18-Induced BMMNC

Interferon gamma (IFNγ) is a proinflammatory cytokine which promotes TH1 polarization during normal development of inflammatory T cell responses. However, chronically high levels of IFNγ have been shown to be myelosuppressive and promote the apoptosis of normal CD34+ stem cell progenitors. High levels of IFNγ, as well as TNFα, have been found to correlate with disease severity in several bone marrow failure syndromes including aplastic anemia, Fanconi anemia and certain subtypes of MDS. Increased IFNγ and TNFα secretions in these diseases have been linked to hyperactivated T lymphocytes as the main inflammatory source of these cytokines. Antigen-mediated activation of IFNγ, such as by anti-CD3 antibody is effectively inhibited by anti-lymphocyte drugs such as cyclosporine A. The induction of IFNγ by anti-CD3 antibody was not inhibited by p38 inhibition with compound 57 (data not shown). Non-antigen activation of IFNγ is found to be mediated by the proinflammatory cytokines IL-12 and IL-18, which in turn, are induced by proinflamamtory stimuli such as LPS. FIG. 33 shows that IL-12 and IL-18 synergistically induced IFNγ in BMMNC after 24 hours and compound 57 potently blocked the IFNγ production with an IC₅₀ of less than 50 nM.

### Compound 57 Reduces MMP-2 and MMP-9 Secretion from BMMNC

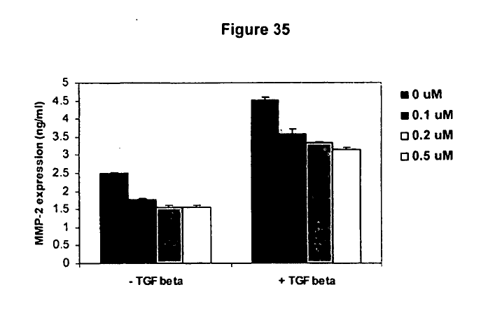

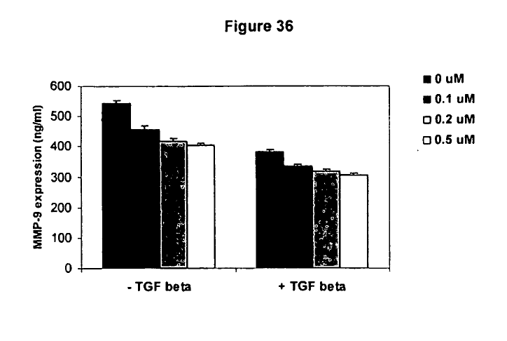

Matrix metalloproteinases (MMPs) are proteases which have been implicated in extracellular matrix (ECM) and basement membrane degradation and in promoting cell migration and invasion in cancer. MMPs are also known to promote the release from the ECM of various factors such as TNFα and VEGF into the bone marrow microenvironment. MMPs such as MMP-2 and MMP-9 have been shown to be secreted by some MDS and by luekemic cells such as AML and B-CLL and have been implicated in tumor cell invasiveness. Since TGF-β is also highly expressed in MDS marrow and has also been known to promote tumor invasiveness in bone metastatic models, we then investigated whether inhibiting p38 MAPK with compound 57 in either basal or TGF-β stimulated BMMNC can lead to decreased production of MMP-2 and MMP-9. FIG. 35 shows that basal as well TGF-β-induced secretion of MMP-2 in BMMNC is reduced by compound 57 in a dose dependent manner. Similarly, compound 57 inhibited the basal level production of MMP-9 in BMMNC (FIG. 36). TGF-β treatment led to a reduction of MMP-9 production in BMMNC. Compound 57, nevertheless led to further reduction of MMP-9 secretion in TGF β-induced BMMNC also in a dose dependent manner.

### Discussion

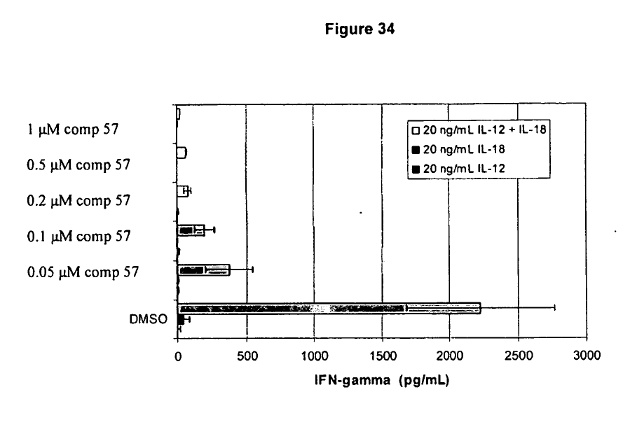

This report demonstrates that compound 57, a selective inhibitor of p38α MAPK, is effective in inhibiting the production of several proinflammatory factors in the bone marrow that have been implicated in the pathobiology of MDS. These factors include inflammatory cytokines such as IL-1β (FIG. 20-22), TNFα (FIG. 23-28) and IFNγ (FIG. 34), all of which have suppressive effects on normal hematopoieisis; inflammatory chemokines induced by TNFα or IL-1β (FIG. 29 and Table 7-8), which recruit and activate cytokine secreting-inflammatory cells to the local site of inflammation (BM); protein factors such as VEGF or IL-6 (FIG. 30-32) which promote MDS disease progression through increased angiogenesis and/or increased MDS blast cell growth and survival; and extracellular matrix (ECM) proteases such as MMP-2 and MMP-9 (FIG. 35-36), both of which contribute to ECM and basement membrane degradation in the bone marrow, to the release of pro-inflammatory cytokines into the microenvironment and to increased tumor invasiveness of transformed leukemic blasts (AML).

The proinflammatory cytokines IL-1β, TNFα and IFNγ, have all been shown to have myelosuppresive effects on the development hematopoietic precursors. TNFα and IFNγ directly induce CD34+ apoptosis through the activation of p38 MAPK. IL-1β, which is secreted by BM macrophages, was found to be secreted by the proliferating abnormal myeloid blasts and appears to be correlated to AML disease severity. Indeed, we found that IL-1β is induced by the inflammatory stimuli LPS, and is inhibited by compound 57 in BM CD34+ cells, in addition to CD14+ monocytes (FIG. 20). Additionally, increased TNFα and IL-1β levels have been correlated to the cause of anemia by suppressing the growth of mature erythroid colony forming units (CFU-E) and by inhibiting the effects of erythroipoietin (Epo) on red blood cell development. IL-1β induces the production of TNFα, and also increases production of PGE₂, both potent suppressors of the myeloid stem cell development. We have shown that IL-1β-induced TNFα expression is regulated by p38 MAPK and inhibited by compound 57 in BM monocytes and T cells. TNFα has also been shown to induce IL-1β, through the activation of NFκB, and TNFα-induced NFκB activation, in turn has been shown to be regulated by p38 MAPK. In addition to regulating transcription, p38 MAPK also regulates the postranscritional modification of TNFα, IL-1β and IFNγ through message stabilization involving MapkapK-2.

TNFα and IL-1β also induces the secretion in BMSC of a number of inflammatory chemokines which we have found to be inhibited by compound 57. These chemokines serve as chemo attractants for leukocytes, particularly monocytes, T cells and granulocytes to the local sites of inflammation, which could lead to the amplification of the inflammatory signal found in chronic inflammation. IFNγ production in BMMNC, which is synergistically induced by IL-12 and IL-18, is almost completely blocked by p38 inhibition with compound 57 (FIG. 34). IL-12 and IL-18 are two proinflammatory cytokines that are produced during inflammation. IL-12 is induced by IL-1β in macrophages and LPS-induced IL-12 is also regulated by p38 MAPK. IL-12 production, together with IL-2, also leads to increased TNFα production. IL-18 expression is correlated with disease severity in AML and it has been shown to increase invasiveness of myeloid leukemic cells through the upregulation of MMP-9 expression. Interestingly, IL-12 and IL-18 also synergistically induce p38-dependent adhesion of T cells to ECM components, a potential downstream effect of IL-1β or TNFα overexpression during inflammation.

The expression of two metalloproteinases, MMP-2/gelatinase A and MMP-9/gelatinase B were found to be reduced by compound 57 in BMMNC. MMP-2 and MMP-9 are upregulated in angiogenic lesions and MMP-9 is involved in the release of VEGF and in promoting the “angiogenic switch” during carcinogenesis. MMP-2 has also been found to be secreted by leukemic blasts and contribute to their invasiveness. Constitutive activation of p38 MAPK has been shown to be critical for MMP-9 production and the survival of B cell chronic lymphocytic leukemia (B-CLL) on bone marrow stromal cells. In fact, non-specific inhibitors of MMPs reduced the apoptosis induction of bone marrow cells in MDS-RA via the inhibition of TNFα. In addition to MDS and other leukemias, the reduction of MMP-2 and MMP-9 by compound 57 in BMMNC may have benefits for other bone related diseases. Inhibiting MMP-9 reduces intraosseous prostate tumor burden and bone degradation in animal models of bone metastasis. TGF-β, which is known to promote bone metastasis, also induces MMP-2 and MMP-9 secretion in some breast cancer cells and inhibiting p38α in breast cancer animal models decreased bone metastasis.

The pleiotropic effects of compound 57 on inhibiting the expression of various proinflammatory factors in the bone marrow, in addition to the disruption of the inflammatory loop that interconnects these factors, leads to the diminution of the suppressive inflammatory signals in the MDS microenvironment to promote normal development of hematopoietic progenitors.

1. A method of preventing apoptosis in myeloid progenitor cells in a MDS patient, comprising:
identifying a subject suffering from MDS; and
administering an effective amount of a p38 MAPK inhibitor, such that MDS-associate dysregulated hematopoiesis is reduced or eliminated.

2. The method of claim 1, wherein apoptosis activity is monitored by caspace activity.

3. A method of inhibiting MDS-promoting cytokine production in bone marrow, comprising:
providing a p38 MAP kinase inhibitor to a subject suffering from MDS, wherein the secretion rate of a MDS-related cytokine is reduced as compared to the secretion rate of a MDS-related cytokine of untreated marrow.

4. The method of claim 3, wherein the MM-related cytokine is selected from the group consisting of IL-6, VEGF, IL-11, and PGE-2.

5. A method of promoting hematopoiesis in a subject suffering from MDS, comprising:
identifying an individual with MDS-associated anemia; and
providing a p38 MAP kinase inhibitor to the individual, such that the MDS-associated anemia is reduced or ameliorated.

6. A method of diagnosing a subject with MDS, comprising:
identifying an individual suspected of suffering from MDS;
obtaining a sample from the individual for testing;
testing the sample for elevated levels of immunosuppressive cytokines relative to a normal control; and
determining whether the individual is presenting elevated levels of immunosuppressive cytokines, which serve as an indicia of MDS.

7. The method of claim 6, wherein the sample is a blood sample.

8. The method of claim 6, wherein the sample is a bone marrow sample.

9. The method of claim 6, wherein the testing step comprises submitting the sample to a cytokine array analysis.

10. The method of claim 6, wherein the testing step comprises submitting the sample to a flow cytometric analysis.
